(19) United States
(12) Patent Application Publication
(10) Pub. No.: US 2026/0117243 A1
(43) Pub. Date: Apr. 30, 2026

(54) Plants Having Increased Tolerance to Herbicides

(71) Applicant: BASF AGRO B.V., Arnhem (NL)

(72) Inventors: Raphael Aponte, Mannheim (DE); Stefan Tresch, Ludwigshafen (DE); Matthias Witschel, Ludwigshafen (DE); Jens Lerchl, Limburgerhof (DE); Jill Marie Paulik, Durham, NC (US); Chad Brommer, Raleigh, NC (US)

(21) Appl. No.: 19/426,940

(22) Filed: Dec. 19, 2025

Related U.S. Application Data
(63) Continuation of application No. 18/157,370, filed on Jan. 20, 2023, now Pat. No. 12,529,066.
Application No. 18/157,370 is a continuation of application No. 16/035,115, filed on Jul. 13, 2018, now Pat. No. 11,572,571.
Application No. 16/035,115 is a continuation of application No. 13/918,212, filed on Jun. 14, 2013, now Pat. No. 10,041,087.
(60) Provisional application No. 61/661,364, filed on Jun. 19, 2012.

(51) Int. Cl. C12N 15/82; A01N 43/84; C12N 9/02; G01N 33/50

(52) U.S. Cl. CPC C12N 15/8274; A01N 43/84; C12N 9/0004; C12N 9/0059; C12Y 103/03004; G01N 33/5097; G01N 2333/90206

(57) ABSTRACT

The present invention refers to a method for controlling undesired vegetation at a plant cultivation site, the method comprising the steps of providing, at said site, a plant that comprises at least one nucleic acid comprising a nucleotide sequence encoding a wild-type or a mutated protoporphyrinogen oxidase (PPO) which is resistant or tolerant to a benzoxazinone-derivative herbicide by applying to said site an effective amount of said herbicide. The invention further refers to plants comprising wild-type or mutated PPO enzymes, and methods of obtaining such plants.

### CROSS-REFERENCE TO RELATED APPLICATIONS

This application is a continuation of U.S. patent application Ser. No. 18/157,370, filed Jan. 20, 2023, which is a continuation of U.S. patent application Ser. No. 16/035,115, filed Jul. 13, 2018, which is a continuation of U.S. patent application Ser. No. 13/918,212, filed Jun. 14, 2013, which claims the benefit of U.S. Provisional Application No. 61/661,364, filed Jun. 19, 2012. The entire contents of the aforementioned applications are incorporated herein by reference.

### REFERENCE TO SEQUENCE LISTING SUBMITTED ELECTRONICALLY

This application was filed electronically via Patent Center and includes an electronically submitted sequence listing in .xml format. The .xml file contains a sequence listing entitled “73943CON1A_Seqlisting.XML” created on Nov. 30, 2022, and is 90,885 bytes in size. The sequence listing contained in this .xml file is part of the specification and is hereby incorporated by reference herein in its entirety.

### FIELD OF THE INVENTION

The present invention relates in general to methods for conferring on plants agricultural level tolerance to a herbicide. Particularly, the invention refers to plants having an increased tolerance to “benzoxazinone-derivative” herbicides. More specifically, the present invention relates to methods and plants obtained by mutagenesis and cross-breeding and transformation that have an increased tolerance to “benzoxazinone-derivative” herbicides.

### BACKGROUND OF THE INVENTION

Herbicides that inhibit protoporphyrinogen oxidase (hereinafter referred to as Protox or PPO; EC: 1.3.3.4), a key enzyme in the biosynthesis of protoporphyrin IX, have been used for selective weed control since the 1960s. PPO catalyzes the last common step in chlorophyll and heme biosynthesis which is the oxidation of protoporphyrinogen IX to protoporphyrin IX. (Matringe et al. 1989. Biochem. 1. 260: 231). PPO-inhibiting herbicides include many different structural classes of molecules (Duke et al. 1991. Weed Sci. 39: 465; Nandihalli et al. 1992. Pesticide Biochem. Physiol. 43: 193; Matringe et al. 1989. FEBS Lett. 245: 35; Yanase and Andoh. 1989. Pesticide Biochem. Physiol. 35: 70). These herbicidal compounds include the diphenylethers {e.g. lactofen, (+−)-2-ethoxy-1-methyl-2-oxoethyl 5-{2-chloro-4-(trifluoromethyl)phenoxy}-2-nitrobenzoate; acifluorfen, 5-{2-chloro-4-(trifluoromethyl)phenoxy}-2-nitrobenzoic acid; its methyl ester; or oxyfluorfen, 2-chloro-1-(3-ethoxy-4-nitrophenoxy)-4-(trifluorobenzene)}, oxidiazoles, (e.g. oxidiazon, 3-{2,4-dichloro-5-(1-methylethoxy)phenyl}-5-(1,1-dimethylethyl)-1,3,4-oxadiazol-2-(3H)-one), cyclic imides (e.g. S-23142, N-(4-chloro-2-fluoro-5-propargyloxyphenyl)-3,4,5,6-tetrahydrophthalimide; chlorophthalim, N-(4-chlorophenyl)-3,4,5,6-tetrahydrophthalimide), phenyl pyrazoles (e.g. TNPP-ethyl, ethyl 2-{1-(2,3,4-trichlorophenyl)-4-nitropyrazolyl-5-oxy}propionate; M&B 39279), pyridine derivatives (e.g. LS 82-556), and phenopylate and its O-phenylpyrrolidino- and piperidinocarbamate analogs. Many of these compounds competitively inhibit the normal reaction catalyzed by the enzyme, apparently acting as substrate analogs.

Application of PPO-inhibiting herbicides results in the accumulation of protoporphyrinogen IX in the chloroplast and mitochondria, which is believed to leak into the cytosol where it is oxidized by a peroxidase. When exposed to light, protoporphyrin IX causes formation of singlet oxygen in the cytosol and the formation of other reactive oxygen species, which can cause lipid peroxidation and membrane disruption leading to rapid cell death (Lee et al. 1993. Plant Physiol. 102:881).

Not all PPO enzymes are sensitive to herbicides which inhibit plant PPO enzymes. Both the Escherichia coli and Bacillus subtilis PPO enzymes (Sasarmen et al. 1993. Can. J. Microbiol. 39:1155; Dailey et al. 1994. J. Biol. Chem. 269:813) are resistant to these herbicidal inhibitors. Mutants of the unicellular alga Chlamydomonas reinhardtii resistant to the phenylimide herbicide S-23142 have been reported (Kataoka et al. 1990. J. Pesticide Sci. 15:449; Shibata et al. 1992. In Research in Photosynthesis, Vol. III, N. Murata, ed. Kluwer: Netherlands. pp. 567-70). At least one of these mutants appears to have an altered PPO activity that is resistant not only to the herbicidal inhibitor on which the mutant was selected, but also to other classes of protox inhibitors (Oshio et al. 1993. Z. Naturforsch. 48c: 339; Sato et al. 1994. In ACS Symposium on Porphyric Pesticides, S. Duke, ed. ACS Press: Washington, D.C.). A mutant tobacco cell line has also been reported that is resistant to the inhibitor S-21432 (Che et al. 1993. Z. Naturforsch. 48c: 350). Auxotrophic E. coli mutants have been used to confirm the herbicide resistance of cloned plant PPOs.

Three main strategies are available for making plants tolerant to herbicides, i.e. (1) detoxifying the herbicide with an enzyme which transforms the herbicide, or its active metabolite, into non-toxic products, such as, for example, the enzymes for tolerance to bromoxynil or to basta (EP242236, EP337899); (2) mutating the target enzyme into a functional enzyme which is less sensitive to the herbicide, or to its active metabolite, such as, for example, the enzymes for tolerance to glyphosate (EP293356, Padgette S. R. et al., J. Biol. Chem., 266, 33, 1991); or (3) overexpressing the sensitive enzyme so as to produce quantities of the target enzyme in the plant which are sufficient in relation to the herbicide, in view of the kinetic constants of this enzyme, so as to have enough of the functional enzyme available despite the presence of its inhibitor. The third strategy was described for successfully obtaining plants which were tolerant to PPO inhibitors (see e.g. U.S. Pat. No. 5,767,373 or 5,939,602, and patent family members thereof.). In addition, US 2010/0100988 and WO 2007/024739 discloses nucleotide sequences encoding amino acid sequences having enzymatic activity such that the amino acid sequences are resistant to PPO inhibitor herbicidal chemicals, in particular 3-phenyluracil inhibitor specific PPO mutants.

To date, the prior art has not described benzoxazinone-derivative herbicide tolerant plants containing at least one wild-type or mutated PPO nucleic acid. Nor has the prior art described benzoxazinone-derivative herbicide tolerant crop plants containing mutations on genomes other than the genome from which the PPO gene is derived. Therefore, what is needed in the art is the identification of benzoxazinone-derivative herbicide tolerance genes from additional genomes and species. What are also needed in the art are crop plants and crop plants having increased tolerance to herbicides such as benzoxazinone-derivative herbicide and containing at least one wildtype and/or mutated PPO nucleic acid. Also needed are methods for controlling weed growth in the vicinity of such crop plants or crop plants. These compositions and methods would allow for the use of spray over techniques when applying herbicides to areas containing crop plants or crop plants.

### SUMMARy OF THE INVENTION

The problem is solved by the present invention which refers to a method for controlling undesired vegetation at a plant cultivation site, the method comprising the steps of:
a) providing, at said site, a plant that comprises at least one nucleic acid comprising a nucleotide sequence encoding a wild type protoporphyrinogen oxidase (PPO) or a mutated protoporphyrinogen oxidase (mut-PPO) which is resistant or tolerant to a benzoxazinone-derivative herbicide,
b) applying to said site an effective amount of said herbicide.

In addition, the present invention refers to a method for identifying a benzoxazinone-derivative herbicide by using a wild-type or mut-PPO encoded by a nucleic acid which comprises the nucleotide sequence of SEQ ID NO: 1, 3, 5, 7, 9, 11, 13, 15, 17, 19, 21, 23, 25, 27, 29, 31, 33, 35, 37, 39, 41, 43, or 45, or a variant thereof.

Said method comprises the steps of:
a) generating a transgenic cell or plant comprising a nucleic acid encoding a mut-PPO, wherein the mut-PPO is expressed;
b) applying a benzoxazinone-derivative herbicide to the transgenic cell or plant of a) and to a control cell or plant of the same variety;
c) determining the growth or the viability of the transgenic cell or plant and the control cell or plant after application of said test compound, and
d) selecting test compounds which confer reduced growth to the control cell or plant as compared to the growth of the transgenic cell or plant.

Another object refers to a method of identifying a nucleotide sequence encoding a mut-PPO which is resistant or tolerant to a benzoxazinone-derivative herbicide, the method comprising:
a) generating a library of mut-PPO-encoding nucleic acids,
b) screening a population of the resulting mut-PPO-encoding nucleic acids by expressing each of said nucleic acids in a cell or plant and treating said cell or plant with a benzoxazinone-derivative herbicide,
c) comparing the benzoxazinone-derivative herbicide-tolerance levels provided by said population of mut-PPO encoding nucleic acids with the benzoxazinone-derivative herbicide-tolerance level provided by a control PPO-encoding nucleic acid,
d) selecting at least one mut-PPO-encoding nucleic acid that provides a significantly increased level of tolerance to a benzoxazinone-derivative herbicide as compared to that provided by the control PPO-encoding nucleic acid.

In a preferred embodiment, the mut-PPO-encoding nucleic acid selected in step d) provides at least 2-fold as much tolerance to a benzoxazinone-derivative herbicide as compared to that provided by the control PPO-encoding nucleic acid.

The resistance or tolerance can be determined by generating a transgenic plant comprising a nucleic acid sequence of the library of step a) and comparing said transgenic plant with a control plant.

Another object refers to a method of identifying a plant or algae containing a nucleic acid encoding a mut-PPO which is resistant or tolerant to a benzoxazinone-derivative herbicide, the method comprising:
a) identifying an effective amount of a benzoxazinone-derivative herbicide in a culture of plant cells or green algae.
b) treating said plant cells or green algae with a mutagenizing agent,
c) contacting said mutagenized cells population with an effective amount of benzoxazinone-derivative herbicide, identified in a),
d) selecting at least one cell surviving these test conditions,
e) PCR-amplification and sequencing of PPO genes from cells selected in d) and comparing such sequences to wild-type PPO gene sequences, respectively.

In a preferred embodiment, the mutagenizing agent is ethylmethanesulfonate.

Another object refers to an isolated nucleic acid encoding a mut-PPO, the nucleic acid comprising the sequence of SEQ ID NO: 1, 3, 5, 7, 9, 11, 13, 15, 17, 19, 21, 23, 25, 27, 29, 31, 33, 35, 37, 39, 41, 43, or 45, or a variant thereof, as defined hereinafter.

Another object refers to an isolated mut-PPO polypeptide, the polypeptide comprising the sequence set forth in SEQ ID NO: 2, 4, 6, 8, 10, 12, 14, 16, 18, 20, 22, 24, 26, 28, 30, 32, 34, 36, 38, 40, 42, 44, or 46, a variant, derivative, orthologue, paralogue or homologue thereof, as defined hereinafter.

In a preferred embodiment, the nucleic acid being identifiable by a method as defined above.

In another embodiment, the invention refers to a plant cell transformed by a wild-type or a mut-PPO nucleic acid or a plant which has been mutated to obtain a plant expressing, preferably over-expressing a wild-type or a mut-PPO nucleic acid, wherein expression of the nucleic acid in the plant cell results in increased resistance or tolerance to a benzoxazinone-derivative herbicide as compared to a wild type variety of the plant cell.

In another embodiment, the invention refers to a plant comprising a plant cell according to the present invention, wherein expression of the nucleic acid in the plant results in the plant's increased resistance to benzoxazinone-derivative herbicide as compared to a wild type variety of the plant.

The plants of the present invention can be transgenic or non-transgenic.

Preferably, the expression of the nucleic acid of the invention in the plant results in the plant's increased resistance to benzoxazinone-derivative herbicide as compared to a wild type variety of the plant.

In another embodiment, the invention refers to a seed produced by a transgenic plant comprising a plant cell of the present invention, wherein the seed is true breeding for an increased resistance to a benzoxazinone-derivative herbicide as compared to a wild type variety of the seed.

In another embodiment, the invention refers to a method of producing a transgenic plant cell with an increased resistance to a benzoxazinone-derivative herbicide as compared to a wild type variety of the plant cell comprising, transforming the plant cell with an expression cassette comprising a wild-type or a mut-PPO nucleic acid.

In another embodiment, the invention refers to a method of producing a transgenic plant comprising, (a) transforming a plant cell with an expression cassette comprising a wild-type or a mut-PPO nucleic acid, and (b) generating a plant with an increased resistance to benzoxazinone-derivative herbicide from the plant cell.

Preferably, the expression cassette further comprises a transcription initiation regulatory region and a translation initiation regulatory region that are functional in the plant.

In another embodiment, the invention relates to using the mut-PPO of the invention as selectable marker. The invention provides a method of identifying or selecting a transformed plant cell, plant tissue, plant or part thereof comprising a) providing a transformed plant cell, plant tissue, plant or part thereof, wherein said transformed plant cell, plant tissue, plant or part thereof comprises an isolated nucleic acid encoding a mut-PPO polypeptide of the invention as described hereinafter, wherein the polypeptide is used as a selection marker, and wherein said transformed plant cell, plant tissue, plant or part thereof may optionally comprise a further isolated nucleic acid of interest; b) contacting the transformed plant cell, plant tissue, plant or part thereof with at least one benzoxazinone-derivative inhibiting compound; c) determining whether the plant cell, plant tissue, plant or part thereof is affected by the inhibitor or inhibiting compound; and d) identifying or selecting the transformed plant cell, plant tissue, plant or part thereof.

The invention is also embodied in purified mut-PPO proteins that contain the mutations described herein, which are useful in molecular modeling studies to design further improvements to herbicide tolerance. Methods of protein purification are well known, and can be readily accomplished using commercially available products or specially designed methods, as set forth for example, in Protein Biotechnology, Walsh and Headon (Wiley, 1994).

### BRIEF DESCRIPTION OF THE DRAWINGS

FIG. 1 shows an amino acid sequence alignment of Amaranthus tuberculatus (A. tuberculatus), Amaranthus tuberculatus resistant (A. tuberculatus_R), Arabidopsis thaliana long (A. thaliana_2), Spinacia oleracea short (S. oleracea_2), Nicotiana tabacum short (N. tabacum_2), Glycine max (Glycine_max), Arabidopsis thaliana short (A. thaliana_1), Nicotiana tabacum long (N. tabacum_1), Chlamydomonas reinhardtii long (C. reinhardtii_1), Zea mays (Z. mays), Oryza sativa (O. sativa_1), Solanum tuberosum (S. tuberosum), Cucumis sativus (C. sativus), Cichorium intybus (C. intybus_1), Spinacia oleracea long (S. oleracea_1), Polytomella sp. Pringsheim 198.80 (Polytomella) PPO sequences. Conserved regions are indicated in light grey, grey and black.

FIG. 2A and FIG. 2B show the selection of Chlamydomonas reinhardtii strains resistant to benzoxazinone-derivative 1.a.35 herbicide. FIG. 2A Mutagenized cells plated on solid medium without a selecting agent. FIG. 2B Mutagenized cells plated on solid medium containing 1×10⁻⁷ M benzoxazinone-derivative I.a.35. Cells which are resistant to the benzoxazinone-derivative herbicide form colonies (circled and numbered 31 and 32), while susceptible cells do not grow. The higher number of colonies on plate A (FIG. 2A) as compared to B (FIG. 2B), indicate that the colonies on plate B are resistant to benzoxazinone-derivative I.a.35.

FIG. 3A and FIG. 3B show growth-characteristics of selected Chlamydomonas reinhardtii strains as seen in FIG. 2A and FIG. 2B, resistant to benzoxazinone-derivative 1.a.35 herbicide. FIG. 3A Dose-response curve of Wild-type cells treated with benzoxazinone-derivative I.a.35 with respective IC₅₀. FIG. 3B Dose-response curve of mutagenized cells (strain 17) treated with benzoxazinone-derivative I.a.35 with respective IC₅₀. Strain 17 (FIG. 3B), resistant to the benzoxazinone-derivative 1.a.35 herbicide, shows much lower IC₅₀, compared to Wild-type cells.

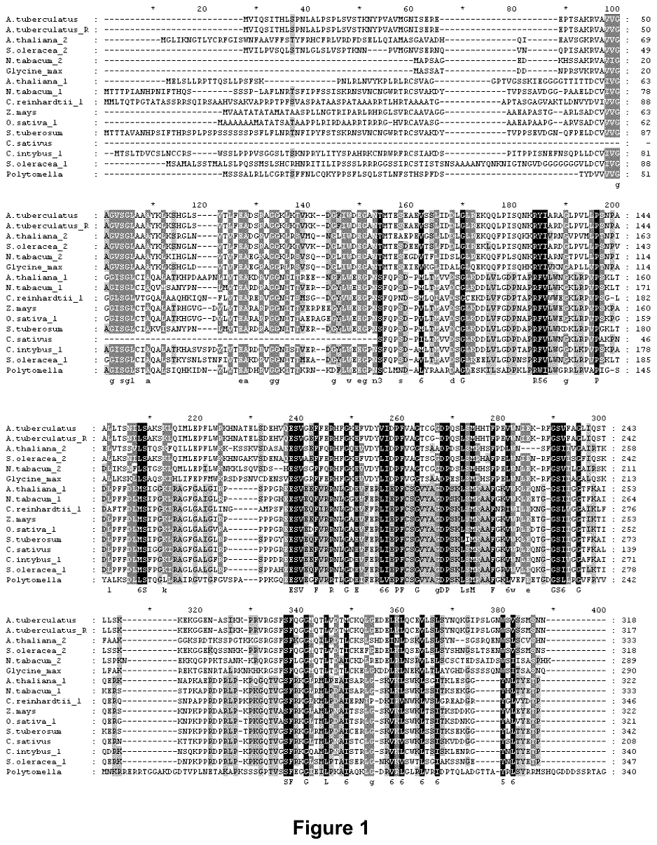

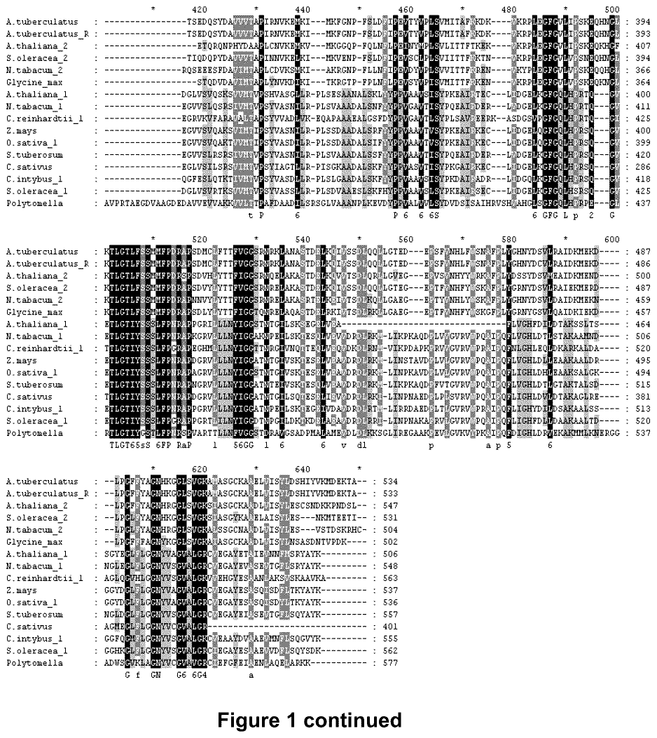

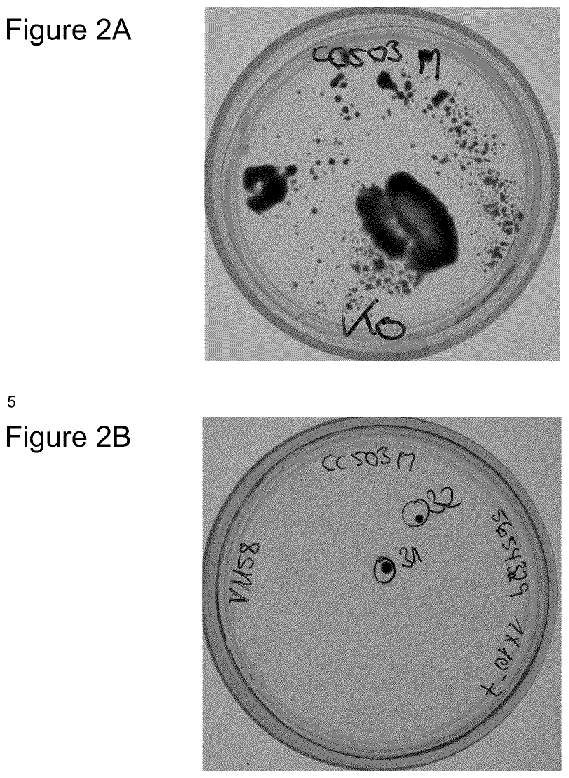

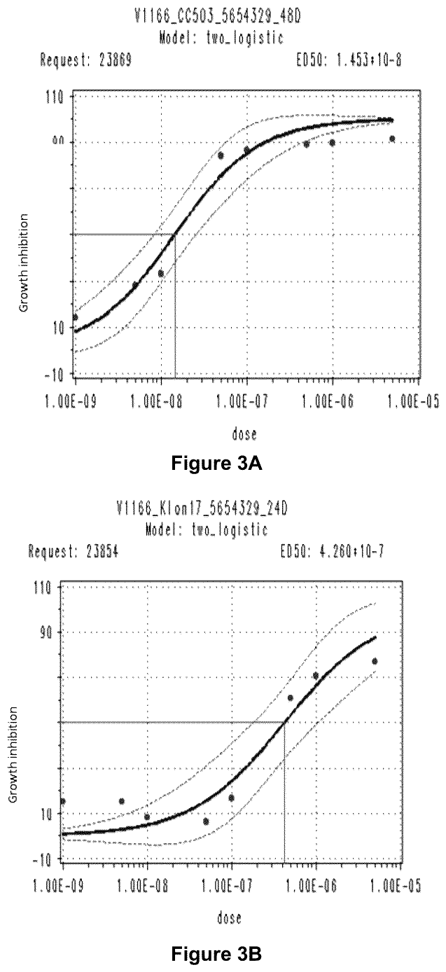

### KEY TO SEQUENCE LISTING

TABLE 1

| SEQ. | | | | |
|---|---|---|---|---|
| ID NO: | Description | Organism | Gene | Accession No: |
| 1 | PPO nucleic acid | Amaranthus | PPX2L_WC | DQ386114 |
| 2 | PPO amino acid | Amaranthus | ABD52326 | |
| 3 | PPO nucleic acid | Amaranthus | PPX2L_AC | DQ386117 |
| 4 | PPO amino acid | Amaranthus | ABD52329 | |
| 5 | PPO nucleic acid | Amaranthus | PPX2L_CC_R | DQ386118 |
| 6 | PPO amino acid | Amaranthus | ABD52330 | |
| 7 | PPO nucleic acid | Amaranthus | PPX2L_AC_R | DQ386116 |
| 8 | PPO amino acid | Amaranthus | ABD52328 | |
| 9 | PPO nucleic acid | Arabidopsis | PPX | AB007650 |
| 10 | PPO amino acid | Arabidopsis | BAB08301 | |
| 11 | PPO nucleic acid | Nicotiana | ppxl | AF044128 |
| 12 | PPO amino acid | Nicotiana | AAD02290 | |
| 13 | PPO nucleic acid | Cichorium | PPX1 | AF160961 |
| 14 | PPO amino acid | Cichorium | AF160961_1 | |
| 15 | PPO nucleic acid | Spinacia | SO-POX1 | AB029492 |
| 16 | PPO amino acid | Spinacia | BAA96808 | |
| 17 | PPO nucleic acid | Spinacia | SO-POX2 | AB046993 |
| 18 | PPO amino acid | Spinacia | BAB60710 | |
| 19 | PPO nucleic acid | Solanum | PPOX | AJ225107 |
| 20 | PPO amino acid | Solanum | CAA12400 | |
| 21 | PPO nucleic acid | Zea | ZM_BFc0091B03 | BT063659 |
| 22 | PPO amino acid | Zea | ACN28356 | |
| 23 | PPO nucleic acid | Zea | prpo2 | NM_001111534 |
| 24 | PPO amino acid | Zea | NP_001105004 | |
| 25 | PPO nucleic acid | Chlamydomonas | Ppx1 | AF068635 |
| 26 | PPO amino acid | Chlamydomonas | AAC79685 | |
| 27 | PPO nucleic acid | Polytomella | PPO | AF332964 |
| 28 | PPO amino acid | Polytomella | AF332964_1 | |
| 29 | PPO nucleic acid | Sorghum | Hyp. Protein | XM_002446665 |
| 30 | PPO amino acid | Sorghum | XP_002446710 | |
| 31 | PPO nucleic acid | Chlorella | | |
| 32 | PPO amino acid | Chlorella | | 51538 |
| 33 | PPO nucleic acid | Oryza | PPOX1 | AB057771 |
| 34 | PPO amino acid | Oryza | BAB39760 | |
| 35 | PPO nucleic acid | Amaranthus | PPX2 | DQ386113 |
| 36 | PPO amino acid | Amaranthus | ABD52325 | |
| 37 | PPO nucleic acid | Arabidopsis | PPOX | NM_178952 |
| 38 | PPO amino acid | Arabidopsis | NP_849283 | |
| 39 | PPO nucleic acid | Nicotiana | ppxll | AF044129 |
| 40 | PPO amino acid | Nicotiana | AAD02291 | |
| 41 | PPO nucleic acid | Glycine | hemG | AB025102 |
| 42 | PPO amino acid | Glycine | BAA76348 | |
| 43 | PPO nucleic acid | Cucumis | CsPPO | AB512426 |
| 44 | PPO amino acid | Cucumis | BAH84864.1 | |
| 45 | PPO nucleic acid | Oryza | Hyp. Protein | AL606613 |
| 46 | PPO amino acid | Oryza | CAE01661 | |

### DETAILED DESCRIPTION

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The present invention refers to a method for controlling undesired vegetation at a plant cultivation site, the method comprising the steps of:
a) providing, at said site, a plant that comprises at least one nucleic acid comprising a nucleotide sequence encoding a wild-type protoporphyrinogen oxidase or a mutated protoporphyrinogen oxidase (mut-PPO) which is resistant or tolerant to a benzoxazinone-derivative herbicide,
b) applying to said site an effective amount of said herbicide.

The term “control of undesired vegetation” is to be understood as meaning the killing of weeds and/or otherwise retarding or inhibiting the normal growth of the weeds. Weeds, in the broadest sense, are understood as meaning all those plants which grow in locations where they are undesired. The weeds of the present invention include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

The term “plant” is used in its broadest sense as it pertains to organic material and is intended to encompass eukaryotic organisms that are members of the Kingdom Plantae, examples of which include but are not limited to vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). The term “plant” further encompasses whole plants, ancestors and progeny of the plants and plant parts, including seeds, shoots, stems, leaves, roots (including tubers), flowers, florets, fruits, pedicles, peduncles, stamen, anther, stigma, style, ovary, petal, sepal, carpel, root tip, root cap, root hair, leaf hair, seed hair, pollen grain, microspore, cotyledon, hypocotyl, epicotyl, xylem, phloem, parenchyma, endosperm, a companion cell, a guard cell, and any other known organs, tissues, and cells of a plant, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. Further preferably, the plant is a monocotyledonous plant, such as sugarcane. Further preferably, the plant is a cereal, such as rice, maize, wheat, barley, millet, rye, sorghum or oats.

In a preferred embodiment, the plant has been previously produced by a process comprising recombinantly preparing a plant by introducing and over-expressing a wild-type or mut-PPO transgene, as described in greater detail hereinafter.

In another preferred embodiment, the plant has been previously produced by a process comprising in situ mutagenizing plant cells, to obtain plant cells which express a mut-PPO.

As disclosed herein, the nucleic acids of the invention find use in enhancing the herbicide tolerance of plants that comprise in their genomes a gene encoding a herbicide-tolerant wild-type or mut-PPO protein. Such a gene may be an endogenous gene or a transgene, as described hereinafter. Additionally, in certain embodiments, the nucleic acids of the present invention can be stacked with any combination of polynucleotide sequences of interest in order to create plants with a desired phenotype. For example, the nucleic acids of the present invention may be stacked with any other polynucleotides encoding polypeptides having pesticidal and/or insecticidal activity, such as, for example, the Bacillus thuringiensis toxin proteins (described in U.S. Pat. Nos. 5,366,892; 5,747,450; 5,737,514; 5,723,756; 5,593,881; and Geiser et al (1986) Gene 48:109). The combinations generated can also include multiple copies of any one of the polynucleotides of interest.

In a particularly preferred embodiment, the plant comprises at least one additional heterologous nucleic acid comprising a nucleotide sequence encoding a herbicide tolerance enzyme selected, for example, from the group consisting of 5-enolpyruvylshikimate-3-phosphate synthase (EPSPS), Glyphosate acetyl transferase (GAT), Cytochrome P450, phosphinothricin acetyltransferase (PAT), Acetohydroxyacid synthase (AHAS; EC 4.1.3.18, also known as acetolactate synthase or ALS), hydroxyphenyl pyruvate dioxygenase (HPPD), Phytoene desaturase (PD) and dicamba degrading enzymes as disclosed in WO 02/068607, or phenoxyaceticacid- and phenoxypropionicacid-derivative degrading enzymes as disclosed in WO 2008141154 or WO 2005107437.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by benzoxazinone-derivative herbicide of the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the benzoxazinone-derivative herbicide postemergence to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wild-type plant. By “herbicide-tolerant mut-PPO protein” or “herbicide-resistant mut-PPO protein”, it is intended that such a mut-PPO protein displays higher PPO activity, relative to the PPO activity of a wild-type mut-PPO protein, when in the presence of at least one herbicide that is known to interfere with PPO activity and at a concentration or level of the herbicide that is known to inhibit the PPO activity of the wild-type mut-PPO protein. Furthermore, the PPO activity of such a herbicide-tolerant or herbicide-resistant mut-PPO protein may be referred to herein as “herbicide-tolerant” or “herbicide-resistant” PPO activity.

The “benzoxazinone-derivative herbicide” that are useful to carry out the present invention encompasses the benzoxazinones of formula I as depicted in the following:

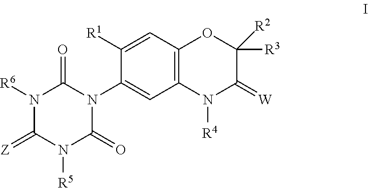

wherein
R¹ is hydrogen or halogen;
R² is halogen;
R³ is hydrogen or halogen;
R⁴ is hydrogen, C₁-C₆-alkyl, C₁-C₆-haloalkyl, C₃-C₆-cycloalkyl, C₃-C₆-alkenyl, C₃-C₆-haloalkenyl, C₃-C₆-alkynyl, C₃-C₆-haloalkynyl, C₁-C₆-alkoxy or C₃-C₆-cycloalkyl-C₁-C₆-alkyl;
R⁵ is hydrogen, NH₂, C₁-C₆-alkyl or C₃-C₆-alkynyl;
R⁶ is hydrogen or C₁-C₆-alkyl; and
W is O or S;
Z is O or S.

In another preferred embodiment, the “benzoxazinone-derivative herbicide” that are useful to carry out the present invention encompasses the benzoxazinones of formula I as depicted in the following:
A) at least one benzoxazinone of the formula I

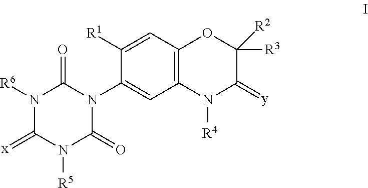

wherein
   R¹ is hydrogen or halogen;
   R² is halogen;
   R³ is hydrogen or halogen;
   R⁴ is hydrogen, C₁-C₆-alkyl, C₁-C₆-haloalkyl, C₃-C₆-cycloalkyl, C₃-C₆-alkenyl, C₃-C₆-haloalkenyl, C₃-C₆-alkynyl, C₃-C₆-haloalkynyl, C₁-C₆-alkoxy or C₃-C₆-cycloalkyl-C₁-C₆-alkyl;
   R⁵ is hydrogen, NH₂, C₁-C₆-alkyl or C₃-C₆-alkynyl;
   R⁶ is hydrogen or C₁-C₆-alkyl; and
   X is O or S;
   Y is O or S;
and at least one further active compound selected from
B) herbicides of class b1) to b15):
   b1) lipid biosynthesis inhibitors;
   b2) acetolactate synthase inhibitors (ALS inhibitors);
   b3 photosynthesis inhibitors;
   b4) protoporphyrinogen-IX oxidase inhibitors,
   b5) bleacher herbicides;
   b6 enolpyruvyl shikimate 3-phosphate synthase inhibitors (EPSP inhibitors);
   b7) glutamine synthetase inhibitors;
   b8) 7,8-dihydropteroate synthase inhibitors (DHP inhibitors);
   b9) mitose inhibitors;
   b10) inhibitors of the synthesis of very long chain fatty acids (VLCFA inhibitors);
   b11) cellulose biosynthesis inhibitors;
   b12) decoupler herbicides;
   b13) auxin herbicides;
   b14) auxin transport inhibitors; and
   b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters;
and
C) safeners.

The benzoxazinone-derivative herbicide that are useful for the present invention can also be compositions in the form of herbicidally active crop protection compositions comprising a herbicidally effective amount of an active compound combination comprising at least one benzoxazinone of formula I and at least one further compound selected from the herbicides B and the safeners C, as defined above, and also at least one liquid and/or solid carrier and/or one or more surfactants and, if desired, one or more further auxiliaries customary for crop protection compositions.

Further, benzoxazinone-derivative herbicide that are useful for the present invention can also be compositions in the form of a crop protection composition formulated as a 1-component composition comprising an active compound combination comprising at least one benzoxazinone of formula I and at least one further active compound selected from the herbicides B and the safeners C, and at least one solid or liquid carrier and/or one or more surfactants and, if desired, one or more further auxiliaries customary for crop protection compositions.

Further, benzoxazinone-derivative herbicide that are useful for the present invention can also be compositions in the form of a crop protection composition formulated as a 2-component composition comprising a first component comprising at least one benzoxazinone of formula I, a solid or liquid carrier and/or one or more surfactants, and a second component comprising at least one further active compound selected from the herbicides B and safeners C, a solid or liquid carrier and/or one or more surfactants, where additionally both components may also comprise further auxiliaries customary for crop protection compositions.

If the benzoxazinones of formula I as described herein are capable of forming geometrical isomers, for example E/Z isomers, it is possible to use both, the pure isomers and mixtures thereof, in the compositions according to the invention.

If the benzoxazinones of formula I as described herein have one or more centers of chirality and, as a consequence, are present as enantiomers or diastereomers, it is possible to use both, the pure enantiomers and diastereomers and their mixtures, in the compositions according to the invention.

The organic moieties mentioned in the definition of the variables R¹ to R⁶, are—like the term halogen—collective terms for individual enumerations of the individual group members. The term halogen denotes in each case fluorine, chlorine, bromine or iodine. All hydrocarbon chains, i.e. all alkyl, can be straight-chain or branched, the prefix Cₙ-Cₘ denoting in each case the possible number of carbon atoms in the group.

Examples of such meanings are:
C₁-C₄-alkyl and also the C₁-C₄-alkyl moieties of C₃-C₆-cycloalkyl-C₁-C₄-alkyl: for example CH₃, C₂H₅, n-propyl, and CH(CH₃)₂ n-butyl, CH(CH₃)—C₂H₅, CH₂—CH(CH₃)₂ and C(CH₃)₃;
C₁-C₆-alkyl and also the C₁-C₆-alkyl moieties of C₁-C₆-alkyoxy-C₁-C₆-alkyl: C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;
C₁-C₄-haloalkyl: a C₁-C₄-alkyl radical as mentioned above which is partially or fully substituted by fluorine, chlorine, bromine and/or iodine, for example, chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, a C₁-C₃-haloalkyl radical as mentioned above, and also, for example, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 1,1,2,2,-tetrafluoroethyl and 1-trifluoromethyl-1,2,2,2-tetrafluoroethyl;
C₁-C₆-haloalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;
C₃-C₆-cycloalkyl and also the cycloalkyl moieties of C₃-C₆-cycloalkyl-C₁-C₄-alkyl: monocyclic saturated hydrocarbons having 3 to 6 ring members, such as cyclopropyl, cyclobutyl, cyclopentyl and cyclohexyl;
C₃-C₆-alkenyl: for example 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
C₃-C₆-haloalkenyl: a C₃-C₆-alkenyl radical as mentioned above which is partially or fully substituted by fluorine, chlorine, bromine and/or iodine, for example 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;
C₃-C₆-alkynyl: for example 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
C₃-C₆-haloalkynyl: a C₃-C₆-alkynyl radical as mentioned above which is partially or fully substituted by fluorine, chlorine, bromine and/or iodine, for example 1,1-difluoroprop-2-yn-1-yl, 3-chloroprop-2-yn-1-yl, 3-bromoprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₁-C₄-alkoxy and also the C₁-C₄-alkoxy moieties of hydroxycarbonyl-C₁-C₄-alkoxy, C₁-C₆-alkoxycarbonyl-C₁-C₄-alkoxy: for example methoxy, ethoxy, propoxy, 1-methylethoxy butoxy, 1-methylpropoxy, 2-methylpropoxy and 1,1-dimethylethoxy;
C₁-C₆-alkoxy and also the C₁-C₆-alkoxy moieties of C₁-C₆-alkoxycarbonyl-C₁-C₄-alkoxy: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy.

According to a preferred embodiment of the invention preference is also given to those benzoxazinone-derivative of formula I, wherein the variables, either independently of one another or in combination with one another, have the following meanings:
R¹ is hydrogen;
   is also preferably halogen, particularly preferred F or Cl, especially preferred F;
R² is F;
R³ is hydrogen or F, preferably hydrogen;
   is also preferably F;
R⁴ is C₃-C₆-alkynyl or C₃-C₆-halolkynyl, preferably C₃-alkynyl or C₃-halolkynyl, particularly preferred CH₂C≡CH, CH₂C≡CCl or CH₂C≡CBr;
   is also preferably C₃-C₆-alkynyl or C₃-C₆-cycloalkyl-C₁-C₆-alkyl, particularly preferred propargyl or cyclopropylmethyl;
   is also preferably C₃-C₆-alkynyl, preferably C₃-alkynyl; particularly preferred CH₂C≡CH;
   is also preferably C₃-C₆-halolkynyl, preferably C₃-halolkynyl, particularly preferred CH₂C≡CCl or CH₂C≡CBr;
R⁵ is NH₂, C₁-C₆-Alkyl or C₃-C₆-alkynyl; preferably C₁-C₆-alkyl; more preferably C₁-C₄-alkyl; most preferably CH₃;
R⁶ is C₁-C₆-alkyl; preferably C₁-C₄-alkyl; most preferably CH₃;
W is O,
   is also preferably S;
Z is O,
   is also preferably S.

Particular preference is given to benzoxazinones of the formula I.a (corresponds to formula I wherein R² is F, R⁵ and R⁶ are CH₃, Wis O and Z is S),

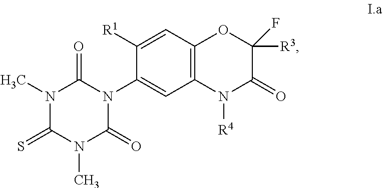

wherein the variables R¹, R³, and R⁴ have the meanings, in particular the preferred meanings, as defined above.

Most preference to the benzoxazinone-derivatives of the formulae I.a.1 to I.a.48 of Table A listed below, in which the variables R¹, R³ and R⁴ together have the meanings given in one row of Table A (benzoxazinones I.a.1 to I.a.54); and where the definitions of the variables R¹, R², R³ and R⁴ are of particular importance for the compounds according to the invention not only in combination with one another but in each case also on their own.

TABLE A

| No. | R¹ | R³ | R⁴ |
|---|---|---|---|
| I.a.1. | H | H | H |
| I.a.2. | H | H | CH₃ |
| I.a.3. | H | H | C₂H₅ |
| I.a.4. | H | H | CH₂—C₂H₅ |
| I.a.5. | H | H | CH(CH₃)₂ |
| I.a.6. | H | H | CH₂—CH₂—(CH₃)₂ |
| I.a.7. | H | H | CH₂—CH═CH₂ |
| I.a.8. | H | H | CH₂CCH |
| I.a.9. | H | H | CH₂CC—Br |
| I.a.10. | H | F | H |
| I.a.11. | H | F | CH₃ |
| I.a.12. | H | F | C₂H₅ |
| I.a.13. | H | F | CH₂—C₂H₅ |
| I.a.14. | H | F | CH(CH₃)₂ |
| I.a.15. | H | F | CH₂—CH₂—(CH₃)₂ |
| I.a.16. | H | F | CH₂—CH═CH₂ |
| I.a.17. | H | F | CH₂CCH |
| I.a.18. | H | F | CH₂CC—Br |
| I.a.19. | F | H | H |
| I.a.20. | F | H | CH₃ |
| I.a.21. | F | H | C₂H₅ |
| I.a.22. | F | H | CH₂—C₂H₅ |
| I.a.23. | F | H | CH(CH₃)₂ |
| I.a.24. | F | H | CH₂—CH₂—(CH₃)₂ |
| I.a.25. | F | H | CH₂—CH═CH₂ |
| I.a.26. | F | H | CH₂CCH |
| I.a.27. | F | H | CH₂CC—Br |
| I.a.28. | F | F | H |
| I.a.29. | F | F | CH₃ |
| I.a.30. | F | F | C₂H₅ |
| I.a.31. | F | F | CH₂—C₂H₅ |
| I.a.32. | F | F | CH(CH₃)₂ |
| I.a.33. | F | F | CH₂—CH₂—(CH₃)₂ |
| I.a.34. | F | F | CH₂—CH═CH₂ |
| I.a.35. | F | F | CH₂CCH |
| I.a.36. | F | F | CH₂CC—Br |
| I.a.37. | Cl | H | H |
| I.a.38. | Cl | H | CH₃ |
| I.a.39. | Cl | H | C₂H₅ |
| I.a.40. | Cl | H | CH₂—C₂H₅ |
| I.a.41. | Cl | H | CH(CH₃)₂ |
| I.a.42. | Cl | H | CH₂—CH₂—(CH₃)₂ |
| I.a.43. | Cl | H | CH₂—CH═CH₂ |
| I.a.44. | Cl | H | CH₂CCH |
| I.a.45. | Cl | H | CH₂CC—Br |
| I.a.46. | Cl | F | H |
| I.a.47. | Cl | F | CH₃ |
| I.a.48. | Cl | F | C₂H₅ |
| I.a.49. | Cl | F | CH₂—C₂H₅ |
| I.a.50. | Cl | F | CH(CH₃)₂ |
| I.a.51. | Cl | F | CH₂—CH₂—(CH₃)₂ |
| I.a.52. | Cl | F | CH₂—CH═CH₂ |
| I.a.53. | Cl | F | CH₂CCH |
| I.a.54. | Cl | F | CH₂CC—Br |

An especially preferred benzoxazinone of the formula I which, as component A, is part of the composition according to the invention, is the benzoxazinone of formula I.a.35 as defined above

According to a particular preferred embodiment of the invention the composition useful for the method of the present invention contains as component A the benzoxazinone of formula I.a.35.

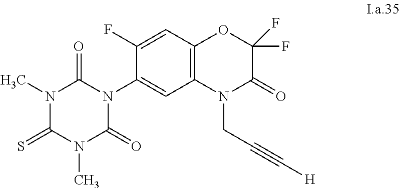 [SMILES: C#CCN1C(=O)C(F)(F)Oc2cc(F)c(-n3c(=O)n(C)c(=S)n(C)c3=O)cc21]

The above described benzoxazinone-derivatives and compositions are disclosed in detail in the European patent application 09163242.2, in particular the disclosures on pages 1 to 7 referring to the bezoxazinone-derivatives and their possible substitutents are entirely incorporated by reference.

The benzoxazinone-derivatives that are useful to carry out the present invention are often best applied in conjunction with one or more other herbicides to obtain control of a wider variety of undesirable vegetation. When used in conjunction with other targeting herbicides, the presently claimed compounds can be formulated with the other herbicide or herbicides, tank mixed with the other herbicide or herbicides, or applied sequentially with the other herbicide or herbicides.

The herbicidal compounds that are useful to carry out the present invention may further be used in conjunction with additional herbicides to which the crop plant is naturally tolerant, or to which it is resistant via expression of one or more additional transgenes as mentioned supra. Some of the herbicides that can be employed in conjunction with the compounds of the present invention include sulfonamides such as metosulam, flumetsulam, cloransulam-methyl, diclosulam, penoxsulam and florasulam, sulfonylureas such as chlorimuron, tribenuron, sulfometuron, nicosulfuron, chlorsulfuron, amidosulfuron, triasulfuron, prosulfuron, tritosulfuron, thifensulfuron, sulfosulfuron and metsulfuron, imidazolinones such as imazaquin, imazapic, ima-zethapyr, imzapyr, imazamethabenz and imazamox, phenoxyalkanoic acids such as 2,4-D, MCPA, dichlorprop and mecoprop, pyridinyloxyacetic acids such as triclopyr and fluroxypyr, carboxylic acids such as clopyralid, picloram, aminopyralid and dicamba, dinitroanilines such as trifluralin, benefin, benfluralin and pendimethalin, chloroacetanilides such as alachlor, acetochlor and metolachlor, semicarbazones (auxin transport inhibitors) such as chlorflurenol and diflufenzopyr, aryloxyphenoxypropionates such as fluazifop, haloxyfop, diclofop, clodinafop and fenoxaprop and other common herbicides including glyphosate, glufosinate, acifluorfen, bentazon, clomazone, fumiclorac, fluometuron, fomesafen, lactofen, linuron, isoproturon, simazine, norflurazon, paraquat, diuron, diflufenican, picolinafen, cinidon, sethoxydim, tralkoxydim, quinmerac, isoxaben, bromoxynil, metribuzin and mesotrione.

For example, the benzoxazinone-derivative herbicides that are useful to carry out the present invention can be used in conjunction with glyphosate and glufosinate on glyphosate-tolerant or glufosinate-tolerant crops.

Unless already included in the disclosure above, the benzoxazinone-derivative herbicides that are useful to carry out the present invention can, further, be used in conjunction with compounds:
b1) from the group of the lipid biosynthesis inhibitors:

ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim and tralkoxydim, and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;
b2) from the group of the ALS inhibitors:

Sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl and tritosulfuron, imidazolinones such as imazamethabenz, imazamethabenz-methyl, imazamox, imazapic, imazapyr, imazaquin and imazethapyr, triazolopyrimidine herbicides and sulfonanilides such as cloransulam, cloransulam-methyl, diclosulam, flumetsulam, florasulam, metosulam, penoxsulam, pyrimisulfan and pyroxsulam,
pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8) and sulfonylaminocarbonyl-triazolinone herbicides such as flucarbazone, flucarbazone-sodium, propoxycarbazon, propoxycarbazon-sodium, thiencarbazone and thiencarbazone-methyl. Among these, a preferred embodiment of the invention relates to those compositions comprising at least one imidazolinone herbicide;
b3) from the group of the photosynthesis inhibitors:
amicarbazone, inhibitors of the photosystem II, e.g. triazine herbicides, including of chlorotriazine, triazinones, triazindiones, methylthiotriazines and pyridazinones such as ametryn, atrazine, chloridazone, cyanazine, desmetryn, dimethametryn, hexazinone, metribuzin, prometon, prometryn, propazin, simazin, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridatre, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;
b4) from the group of the protoporphyrinogen-IX oxidase inhibitors:
acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 45100-03-7) and 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione;
b5) from the group of the bleacher herbicides:
PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)pyrimidine (CAS 180608-33-7), HPPD inhibitors: benzobicyclon, benzofenap, isoxaflutole, mesotrione, pyrasulfotole, pyrazolynate, pyrazoxyfen, sulcotrione, tefuryltrione, tembotrione, topramezone and bicyclopyrone, bleacher, unknown target: aclonifen, amitrole, clomazone and flumeturon;
b6) from the group of the EPSP synthase inhibitors:
glyphosate, glyphosate-isopropylammonium and glyphosate-trimesium (sulfosate);
b7) from the group of the glutamine synthase inhibitors:
bilanaphos (bialaphos), bilanaphos-sodium, glufosinate, glufosinate-P and glufosinate-ammonium;
b8) from the group of the DHP synthase inhibitors:
asulam;
b9) from the group of the mitose inhibitors:
compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlorthal-dimethyl, pyridines such as dithiopyr and thiazopyr, benzamides such as propyzamide and tebutam; compounds of group K2: chlorpropham, propham and carbetamide, among these, compounds of group K1, in particular dinitroanilines are preferred;
b10) from the group of the VLCFA inhibitors:
chloroacetamides such as acetochlor, alachlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, acetanilides such as diphenamid, naproanilide and napropamide, tetrazolinones such fentrazamide, and other herbicides such as anilofos, cafenstrole, fenoxasulfone, ipfencarbazone, piperophos, pyroxasulfone and
isoxazoline compounds of the formula II,

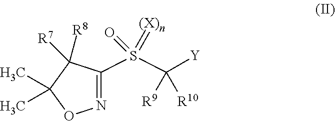

wherein R⁷, R⁸, R⁹, R¹⁰, W, Z and n have the following meanings:
   R⁷, R⁸, R⁹, R¹⁰ independently of one another hydrogen, halogen or C₁-C₄-alkyl;
   X oxygen or NH;
   Y phenyl or monocyclic 5-, 6-, 7-, 8-, 9- or 10-membered heterocyclyl containing, in addition to carbon ring members one, two or three same or different heteroatoms selected from oxygen, nitrogen and sulfur as ring members, wherein phenyl and heterocyclyl are unsubstituted or carry 1, 2 or 3 substituents R^{yy} selected from halogen, C₁-C₄-alkyl, C₁-C₄-alkoxy, C₁-C₄-haloalkyl and C₁-C₄-haloalkoxy;
      preferably phenyl or 5- or 6-membered aromatic heterocyclyl (hetaryl) which contains, in addition to carbon ring members, one, two or three nitrogen atoms as ring members, wherein phenyl and hetaryl are unsubstituted or carry 1, 2 or 3 substituents R^{yy}; and
   n zero or one;
   among the isoxazoline compounds of the formula II, preference is given to isoxazoline compounds of the formula II, wherein
   R⁷, R⁸, R⁹, R¹⁰ independently of one another are H, F, Cl or methyl;
   X is oxygen;
   n is 0 or 1; and
   Y is phenyl, pyrazolyl or 1,2,3-triazolyl, wherein the three last-mentioned radicals are unsubstituted or carry one, two or three substituents R^{yy}, especially one of the following radicals

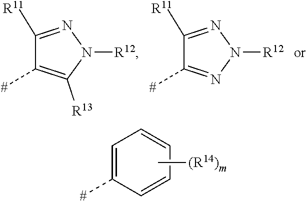

wherein
   R¹¹ is halogen, C₁-C₄-alkyl or C₁-C₄-haloalkyl;
      R¹² is C₁-C₄-alkyl;
      R¹³ is halogen, C₁-C₄-alkoxy or C₁-C₄-haloalkoxy;
      R¹⁴ is halogen, C₁-C₄-alkyl, C₁-C₄-haloalkyl or C₁-C₄-haloalkoxy;
      m is 0, 1, 2 or 3; and
      # denotes the point of attachment to the group CR¹³R¹⁴;
   among the isoxazoline compounds of the formula II, particular preference is given to those isoxazoline compounds of the formula II, wherein
      R⁷ is hydrogen;
      R⁸ is fluorine;
      R⁹ is hydrogen or fluorine;
      R¹⁰ is hydrogen or fluorine;
      X is oxygen;
      Y is one of the radicals of the formulae Y¹, Y², Y³ or Y⁴

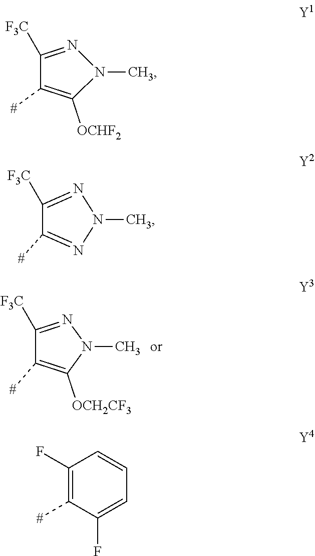

wherein # denotes the point of attachment to the group CR⁹R¹⁰;
   n is zero or 1, in particular 1; and
   among these, especially preferred are the isoxazoline compounds of the formulae II.1, II.2, II.3, II.4, II.5, II.6, II.7, II.8 and II.9

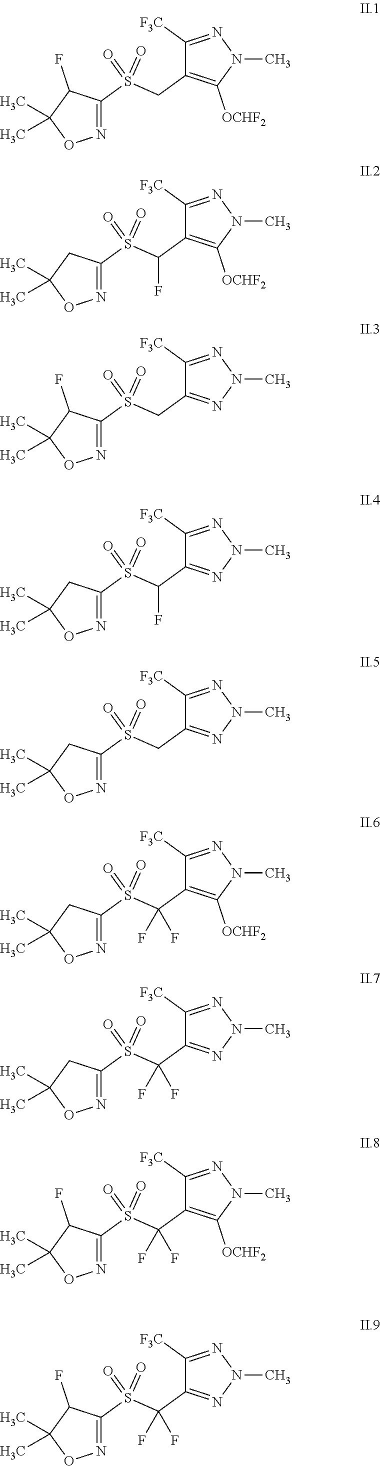

the isoxazoline compounds of the formula II are known in the art, e.g. from WO 2006/024820, WO 2006/037945, WO 2007/071900 and WO 2007/096576;
   among the VLCFA inhibitors, preference is given to chloroacetamides and oxyacetamides, especially to pyroxasulfone;
b11) from the group of the cellulose biosynthesis inhibitors:
chlorthiamid, dichlobenil, flupoxam, isoxaben, 1-Cyclohexyl-5-pentafluorphenyloxy-1⁴-[1,2,4,6]thiatriazin-3-ylamine and piperazine compounds of formula III,

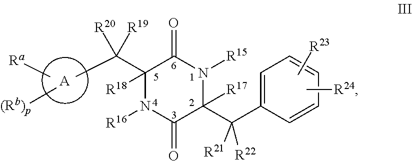

in which
   A is phenyl or pyridyl where R^{a} is attached in the ortho-position to the point of attachment of A to a carbon atom;
   R^{a} is CN, NO₂, C₁-C₄-alkyl, D-C₃-C₆-cycloalkyl, C₁-C₄-haloalkyl, C₁-C₄-alkoxy, C₁-C₄-haloalkoxy, O-D-C₃-C₆-cycloalkyl, S(O)_{q}R^{y}, C₂-C₆-alkenyl, D-C₃-C₆-cycloalkenyl, C₃-C₆-alkenyloxy, C₂-C₆-alkynyl, C₃-C₆-alkynyloxy, NR^{A}R^{B}, tri-C₁-C₄-alkylsilyl, D-C(═O)—R^{a1}, D-P(═O)(R^{a1})₂, phenyl, naphthyl, a 3- to 7-membered monocyclic or 9- or 10-membered bicyclic saturated, unsaturated or aromatic heterocycle which is attached via carbon or nitrogen, which contains 1, 2, 3 or 4 heteroatoms selected from the group consisting of O, N and S, and which may be partially or fully substituted by groups R^{aa} and/or R^{a1}, and, if R^{a} is attached to a carbon atom, additionally halogen;
      R^{y} is C₁-C₆-alkyl, C₃-C₄-alkenyl, C₃-C₄-alkynyl, NR^{A}R^{B} or C₁-C₄-haloalkyl and q is 0, 1 or 2;
      R^{A},R^{B} independently of one another are hydrogen, C₁-C₆-alkyl, C₃-C₆-alkenyl and C₃-C₆-alkynyl; together with the nitrogen atom to which they are attached, R^{A},R^{B} may also form a five- or six-membered saturated, partially or fully unsaturated ring which, in addition to carbon atoms, may contain 1, 2 or 3 heteroatoms selected from the group consisting of O, N and S, which ring may be substituted by 1 to 3 groups R^{aa};
      D is a covalent bond, C₁-C₄-alkylene, C₂-C₆-alkenyl or C₂-C₆-alkynyl;
      R^{a1} is hydrogen, OH, C₁-C₈-Alkyl, C₁-C₄-haloalkyl, C₃-C₆-cycloalkyl, C₂-C₈-alkenyl, C₅-C₆-cycloalkenyl, C₂-C₈-alkynyl, C₁-C₆-alkoxy, C₁-C₄-haloalkoxy, C₃-C₈-alkenyloxy, C₃-C₈-alkynyloxy, NR^{A}R^{B}, C₁-C₆-alkoxyamino, C₁-C₆-alkylsulfonylamino, C₁-C₆-alkylaminosulfonylamino, [di-(C₁-C₆)alkylamino]sulfonylamino, C₃-C₆-alkenylamino, C₃-C₆-alkynylamino, N—(C₂-C₆-alkenyl)-N—(C₁-C₆-alkyl)amino, N—(C₂-C₆-alkynyl)-N—(C₁-C₆-alkyl)amino, N—(C₁-C₆-alkoxy)-N—(C₁-C₆-alkyl)amino, N—(C₂-C₆-alkenyl)-N—(C₁-C₆-alkoxy)amino, N—(C₂-C₆-alkynyl)-N—(C₁-C₆-alkoxy)-amino, C₁-C₆-alkylsulfonyl, tri-C₁-C₄-alkylsilyl, phenyl, phenoxy, phenylamino or a 5- or 6-membered monocyclic or 9- or 10-membered bicyclic heterocycle which contains 1, 2, 3 or 4 heteroatoms selected from the group consisting of O, N and S, where the cyclic groups are unsubstituted or substituted by 1, 2, 3 or 4 groups R^{aa};
      R^{aa} is halogen, OH, CN, NO₂, C₁-C₄-alkyl, C₁-C₄-haloalkyl, C₁-C₄-alkoxy, C₁-C₄-haloalkoxy, S(O)_{q}R^{y}, D-C(═O)—R^{a1} and tri-C₁-C₄-alkylsilyl;
   R^{b} independently of one another are hydrogen, CN, NO₂, halogen, C₁-C₄-alkyl, C₁-C₄-haloalkyl, C₂-C₄-alkenyl, C₃-C₆-alkynyl, C₁-C₄-alkoxy, C₁-C₄-haloalkoxy, benzyl or S(O)_{q}R^{y},
      R^{b} together with the group R^{a} or R^{b} attached to the adjacent ring atom may also form a five- or six-membered saturated or partially or fully unsaturated ring which, in addition to carbon atoms, may contain 1, 2 or 3 heteroatoms selected from the group consisting of O, N and S, which ring may be partially or fully substituted by R^{aa};
   p is 0, 1, 2 or 3;
   R¹⁵ is hydrogen, OH, CN, C₁-C₁₂-alkyl, C₃-C₁₂-alkenyl, C₃-C₁₂-alkynyl, C₁-C₄-alkoxy, C₃-C₆-cycloalkyl, C₅-C₆-cycloalkenyl, NR^{A}R^{B}, S(O)ₙR^{y}, S(O)ₙNR^{A}R^{B}, C(═O)R²⁵, CONR^{A}R^{B}, phenyl or a 5- or 6-membered monocyclic or 9- or 10-membered bicyclic aromatic heterocycle which contains 1, 2, 3 or 4 heteroatoms selected from the group consisting of O, N and S, where the cyclic groups are attached via D¹ and are unsubstituted or substituted by 1, 2, 3 or 4 groups R^{aa}, and also the following partially or fully R^{aa}-substituted groups: C₁-C₄-alkyl, C₃-C₄-alkenyl, C₃-C₄-alkynyl, C₁-C₄-alkoxy, C₃-C₆-cycloalkyl, C₅-C₆-cycloalkenyl, NR^{A}R^{B}, S(O)ₙR^{y}, S(O)ₙNR^{A}R^{B}, C(═O)R²⁵, CONR^{A}R^{B};
      preferably is hydrogen, OH, CN, C₁-C₁₂-alkyl, C₃-C₁₂-alkenyl, C₃-C₁₂-alkynyl, C₁-C₄-alkoxy, C₃-C₆-cycloalkyl, C₅-C₆-cycloalkenyl, NR^{A}R^{B}, S(O)ₙR^{y}, S(O)ₙNR^{A}R^{B}, C(═O)R²⁵, CONR^{A}R^{B}, phenyl or a 5- or 6-membered monocyclic or 9- or 10-membered bicyclic aromatic heterocycle which contains 1, 2, 3 or 4 heteroatoms selected from the group consisting of O, N and S, where the cyclic groups are attached via D¹ and are unsubstituted or substituted by 1, 2, 3 or 4 groups R^{aa}, and also the following partially or fully R^{aa}-substituted groups: C₁-C₄-alkyl, C₃-C₄-alkenyl and C₃-C₄-alkynyl;
      R²⁵ is hydrogen, C₁-C₄-alkyl, C₁-C₄-haloalkyl, C₁-C₄-alkoxy or C₁-C₄-haloalkoxy;
      D¹ is carbonyl or a group D;
      where in groups R¹⁵, R^{a} and their sub-substituents the carbon chains and/or the cyclic groups may carry 1, 2, 3 or 4 substituents R^{aa} and/or R^{a1};
   R¹⁶ is C₁-C₄-alkyl, C₃-C₄-alkenyl or C₃-C₄-alkynyl;
   R¹⁷ is OH, NH₂, C₁-C₄-alkyl, C₃-C₆-cycloalkyl, C₃-C₆-alkenyl, C₃-C₆-alkynyl, C₁-C₄-hydroxyalkyl, C₁-C₄-cyanoalkyl, C₁-C₄-haloalkyl, C₁-C₄-alkoxy-C₁-C₄-alkyl or C(═O)R²⁵;
   R¹⁸ is hydrogen, halogen, C₁-C₄-alkyl or C₁-C₄-haloalkyl, or R¹⁸ and R¹⁹ together are a covalent bond;
   R¹⁹, R²⁰, R²¹, R²¹ independently of one another are hydrogen, halogen, OH, CN, NO₂, C₁-C₄-alkyl, C₁-C₄-haloalkyl, C₂-C₆-alkenyl, C₂-C₆-alkynyl, C₁-C₄-alkoxy, C₁-C₄-haloalkoxy, C₃-C₆-cycloalkyl, C₃-C₆-cycloalkenyl and C₃-C₆-cycloalkynyl;
   R²³, R²⁴ independently of one another are hydrogen, halogen, OH, haloalkyl, NR^{A}R^{B}, NR^{A}C(O)R²⁶, CN, NO₂, C₁-C₄-alkyl, C₁-C₄-haloalkyl, C₂-C₄-alkenyl, C₃-C₆-alkynyl, C₁-C₄-alkoxy, C₁-C₄-haloalkoxy, O—C(O)R²⁶, phenoxy or benzyloxy, where in groups R²³ and R²⁴ the carbon chains and/or the cyclic groups may carry 1, 2, 3 or 4 substituents R^{aa};
   R²⁶ is C₁-C₄-alkyl or NR^{A}R^{B};
   among the isoxazoline compounds of the piperazin compounds of formula III, preference is given to the piperazine compounds of the formula III, wherein
   A is phenyl or pyridyl where R^{a} is attached in the ortho-position to the point of attachment of A to a carbon atom;
   R^{a} is CN, NO₂, C₁-C₄-alkyl, C₁-C₄-haloalkyl, C₁-C₄-alkoxy, C₁-C₄-haloalkoxy or D-C(═O)—R^{a1};
      R^{y} is C₁-C₆-alkyl, C₃-C₄-alkenyl, C₃-C₄-alkynyl, NR^{A}R^{B} or C₁-C₄-haloalkyl and q is 0, 1 or 2;
      R^{A},R^{B} independently of one another are hydrogen, C₁-C₆-alkyl, C₃-C₆-alkenyl and C₃-C₆-alkynyl; together with the nitrogen atom to which they are attached, R^{A},R^{B} may also form a five- or six-membered saturated, partially or fully unsaturated ring which, in addition to carbon atoms, may contain 1, 2 or 3 heteroatoms selected from the group consisting of O, N and S, which ring may be substituted by 1 to 3 groups R^{aa};
      D is a covalent bond or C₁-C₄-alkylene;
      R^{a1} is hydrogen, OH, C₁-C₈-Alkyl, C₁-C₄-haloalkyl, C₃-C₆-cycloalkyl;
      R^{aa} is halogen, OH, CN, NO₂, C₁-C₄-alkyl, C₁-C₄-haloalkyl, C₁-C₄-alkoxy, C₁-C₄-haloalkoxy, S(O)_{q}R^{y}, D-C(═O)—R^{a1} and tri-C₁-C₄-alkylsilyl;
   R^{b} independently of one another is CN, NO₂, halogen, C₁-C₄-alkyl, C₁-C₄-haloalkyl, C₂-C₄-alkenyl, C₃-C₆-alkynyl, C₁-C₄-alkoxy, C₁-C₄-haloalkoxy, benzyl or S(O)_{q}R^{y}, R^{b} together with the group R^{a} or R^{b} attached to the adjacent ring atom may also form a five- or six-membered saturated or partially or fully unsaturated ring which, in addition to carbon atoms, may contain 1, 2 or 3 heteroatoms selected from the group consisting of O, N and S, which ring may be partially or fully substituted by R^{aa};
   p is 0 or 1;
   R¹⁵ is hydrogen, C₁-C₁₂-alkyl, C₃-C₁₂-alkenyl, C₃-C₁₂-alkynyl, C₁-C₄-alkoxy or C(═O)R²⁵, which can be partially or fully substituted by R^{aa}-groups;
      preferably is hydrogen, C₁-C₁₂-alkyl, C₃-C₁₂-alkenyl, C₃-C₁₂-alkynyl, C₁-C₄-alkoxy or C(═O)R²⁵;
      R²⁵ is hydrogen, C₁-C₄-alkyl, C₁-C₄-haloalkyl, C₁-C₄-alkoxy or C₁-C₄-haloalkoxy;
      where in groups R¹⁵, R^{a} and their sub-substituents the carbon chains and/or the cyclic groups may carry 1, 2, 3 or 4 substituents R^{aa} and/or R^{a1};
   R¹⁶ is C₁-C₄-alkyl;
   R¹⁷ is OH, NH₂, C₁-C₄-alkyl, C₃-C₆-cycloalkyl, C₁-C₄-haloalkyl or C(═O)R²⁵;
   R¹⁸ is hydrogen, or R¹⁸ and R¹⁹ together are a covalent bond;
   R¹⁹, R²⁰, R²¹, R²¹ independently of one another are hydrogen;
   R²³, R²⁴ independently of one another are hydrogen, halogen or OH;
b12) from the group of the decoupler herbicides:
dinoseb, dinoterb and DNOC and its salts;
b13) from the group of the auxin herbicides:
2,4-D and its salts and esters, 2,4-DB and its salts and esters, aminopyralid and its salts such as aminopyralid-tris(2-hydroxypropyl) ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, and aminocyclopyrachlor and its salts and esters;
b14) from the group of the auxin transport inhibitors: diflufenzopyr, diflufenzopyr-sodium, naptalam and naptalam-sodium;
b15) from the group of the other herbicides: bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters.

Moreover, it may be useful to apply the benzoxazinones of the formula I in combination with safeners. Safeners are chemical compounds which prevent or reduce damage on useful plants without having a major impact on the herbicidal action of the benzoxazinones of the formula I towards unwanted plants. They can be applied either before sowings (e.g. on seed treatments, shoots or seedlings) or in the pre-emergence application or post-emergence application of the useful plant.

Furthermore, the safeners C, the benzoxazinones I and/or the herbicides B can be applied simultaneously or in succession.

Suitable safeners are e.g. (quinolin-8-oxy) acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

Examples of preferred safeners C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4), and N-(2-Methoxybenzoyl)-4-[(methylaminocarbonyl)amino]benzenesulfonamide (CAS 129531-12-0).

Especially preferred safeners C are benoxacor, cloquintocet, cyprosulfamide, dichlormid, fenchlorazole, fenclorim, flurazole, fluxofenim, furilazole, isoxadifen, mefenpyr, naphthalic anhydride, oxabetrinil, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4), and N-(2-Methoxybenzoyl)-4-[(methylaminocarbonyl)amino]benzenesulfonamide (CAS 129531-12-0).

Particularly preferred safeners C are benoxacor, cloquintocet, cyprosulfamide, dichlormid, fenchlorazole, fenclorim, furilazole, isoxadifen, mefenpyr, 4-(dichloroacetyl)-1-oxa-4-azaspiro-[4.5]decane (MON4660, CAS 71526-07-3) and 2,2,5-trimethyl-3-(dichloroacetyl)-1,3-oxazolidine (R-29148, CAS 52836-31-4), and N-(2-Methoxybenzoyl)-4-[(methylaminocarbonyl)amino]benzenesulfonamide (CAS 129531-12-0).

Particularly preferred safeners C, which, as component C, are constituent of the composition according to the invention are the safeners C as defined above; in particular the safeners C.1-C.13 listed below in table C:

TABLE C
Safener C

C.1 benoxacor
C.2 cloquintocet
C.3 cyprosulfamide
C.4 dichlormid
C.5 fenchlorazole
C.6 fenclorim
C.7 furilazole
C.8 isoxadifen
C.9 mefenpyr
C.10 naphtalic acid anhydride
C.11 4-(dichloroacetyl)-1-oxa-4-azaspiro[4.5]decane
(MON4660, CAS 71526-07-3)
C.12 2,2,5-trimethyl-3-(dichloro-acetyl)-1,3-oxazolidine
(R-29148, CAS 52836-31-4)
C.13 N-(2-Methoxybenzoyl)-
4-[(methylaminocarbonyl)amino]benzenesulfonamide
(CAS 129531-12-0)

The active compounds B of groups b1) to b15) and the active compounds C are known herbicides and safeners, see, for example, The Compendium of Pesticide Common Names (alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660. Further herbicidally active compounds are known from WO 96/26202, WO 97/41116, WO 97/41117, WO 97/41118 and WO 01/83459 and also from W. Krämer et al. (ed.) “Modern Crop Protection Compounds”, Vol. 1, Wiley VCH, 2007 and the literature cited therein.

It is generally preferred to use the compounds of the invention in combination with herbicides that are selective for the crop being treated and which complement the spectrum of weeds controlled by these compounds at the application rate employed. It is further generally preferred to apply the compounds of the invention and other complementary herbicides at the same time, either as a combination formulation or as a tank mix.

It is recognized that the polynucleotide molecules and polypeptides of the invention encompass polynucleotide molecules and polypeptides comprising a nucleotide or an amino acid sequence that is sufficiently identical to nucleotide sequences set forth in SEQ ID Nos: 1, 3, 5, 7, 9, 11, 13, 15, 17, 19, 21, 23, 25, 27, 29, 31, 33, 35, 37, 39, 41, 43, or 45, or to the amino acid sequences set forth in SEQ ID Nos: 2, 4, 6, 8, 10, 12, 14, 16, 18, 20, 22, 24, 26, 28, 30, 32, 34, 36, 38, 40, 42, 44, or 46. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity.

Generally, “sequence identity” refers to the extent to which two optimally aligned DNA or amino acid sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG. Wisconsin Package. (Accelrys Inc. Burlington, Mass.)

### Polynucleotides and Oligonucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. As the skilled addressee would be aware, an isolated polynucleotide can be an exogenous polynucleotide present in, for example, a transgenic organism which does not naturally comprise the polynucleotide. Furthermore, the terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

The term “mut-PPO nucleic acid” refers to a PPO nucleic acid having a sequence that is mutated from a wild-type PPO nucleic acid and that confers increased benzoxazinone-derivative herbicide tolerance to a plant in which it is expressed. Furthermore, the term “mutated protoporphyrinogen oxidase (mut-PPO)” refers to the replacement of an amino acid of the wild-type primary sequences SEQ ID NO: 2, 4, 6, 8, 10, 12, 14, 16, 18, 20, 22, 24, 26, 28, 30, 32, 34, 36, 38, 40, 42, 44, or 46, or a variant, a derivative, a homologue, an orthologue, or paralogue thereof, with another amino acid. The expression “mutated amino acid” will be used below to designate the amino acid which is replaced by another amino acid, thereby designating the site of the mutation in the primary sequence of the protein.

In a preferred embodiment, the PPO nucleotide sequence comprises the sequence of SEQ ID NO: 1, 25, 37 or 39 or a variant or derivative thereof.

Furthermore, it will be understood by the person skilled in the art that the PPO nucleotide sequences encompasse homologues, paralogues and and orthologues of SEQ ID NO: 1, 25, 37 or 39 as defined hereinafter.

The term “variant” with respect to a sequence (e.g., a polypeptide or nucleic acid sequence such as—for example—a transcription regulating nucleotide sequence of the invention) is intended to mean substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO: SEQ ID NO: 1, 3, 5, 7, 9, 11, 13, 15, 17, 19, 21, 23, 25, 27, 29, 31, 33, 35, 37, 39, 41, 43, or 45. The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

### Polypeptides

By “substantially purified polypeptide” or “purified” a polypeptide is meant that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. As the skilled addressee will appreciate, the purified polypeptide can be a recombinantly produced polypeptide. The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the invention as described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

With regard to a defined polypeptide, it will be appreciated that % identity figures higher than those provided above will encompass preferred embodiments. Thus, where applicable, in light of the minimum % identity figures, it is preferred that the PPO polypeptide of the invention comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to SEQ ID NO: 2, 4, 6, 8, 10, 12, 14, 16, 18, 20, 22, 24, 26, 28, 30, 32, 34, 36, 38, 40, 42, 44, or 46.

By “variant” polypeptide is intended a polypeptide derived from the protein of SEQ ID NO: 2, 4, 6, 8, 10, 12, 14, 16, 18, 20, 22, 24, 26, 28, 30, 32, 34, 36, 38, 40, 42, 44, or 46 by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds).

TABLE 2
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, OH), QuickChange Site Directed mutagenesis (Stratagene, San Diego, CA), PCR-mediated site-directed mutagenesis or other site-directed mutagenesis protocols.

“Derivatives” further include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. A non-limiting list of examples of such orthologues are shown in Table 1.

It is well-known in the art that paralogues and orthologues may share distinct domains harboring suitable amino acid residues at given sites, such as binding pockets for particular substrates or binding motifs for interaction with other proteins.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48:443-453) to find the global (i.e. spanning the complete sequences) alignment of two sequences that maximizes the number of matches and minimizes the number of gaps. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

The inventors of the present invention have found that by substituting one or more of the key amino acid residues the herbicide tolerance or resistance could be remarkably increased as compared to the activity of the wild type PPO enzymes with SEQ ID NO: 2, 4, 6, 8, 10, 12, 14, 16, 18, 20, 22, 24, 26, 28, 30, 32, 34, 36, 38, 40, 42, 44, or 46. Preferred substitutions of mut-PPO are those that increase the herbicide tolerance of the plant, but leave the biological activity of the oxidase activity substantially unaffected.

Accordingly, in another object of the present invention the key amino acid residues of a PPO enzyme, a variant, derivative, orthologue, paralogue or homologue thereof, is substituted by any other amino acid.

In a preferred embodiment, the key amino acid residues of a PPO enzyme, a variant, derivative, orthologue, paralogue or homologue thereof, is substituted by a conserved amino acid as depicted in Table 2.

It will be understood by the person skilled in the art that amino acids located in a close proximity to the positions of amino acids mentioned below may also be substituted. Thus, in another embodiment the variant of SEQ ID NO: 2, 4, 6, 8, 10, 12, 14, 16, 18, 20, 22, 24, 26, 28, 30, 32, 34, 36, 38, 40, 42, 44, or 46, a variant, derivative, orthologue, paralogue or homologue thereof comprises a mut-PPO, wherein an amino acid ±3, ±2 or ±1 amino acid positions from a key amino acid is substituted by any other amino acid.

Based on techniques well-known in the art, a highly characteristic sequence pattern can be developed, by means of which further of mut-PPO candidates with the desired activity may be searched.

Searching for further mut-PPO candidates by applying a suitable sequence pattern would also be encompassed by the present invention. It will be understood by a skilled reader that the present sequence pattern is not limited by the exact distances between two adjacent amino acid residues of said pattern. Each of the distances between two neighbours in the above patterns may, for example, vary independently of each other by up to ±10, ±5, ±3, ±2 or ±1 amino acid positions without substantially affecting the desired activity.

Furthermore, by applying the method of site directed mutagenesis, the inventors of the present invention have identified specific combinations of mutations, which combination refers to a substitution of the Phenylalanine residue at position 420 in SEQ ID NO:2 or 4, combined with a second substitution of the Leucin at position 397 in SEQ ID NO:2 or 4.

Thus, in a particularly preferred embodiment, the variant or derivative of the mut-PPO of SEQ ID NO: 2 or SEQ ID NO: 4 is selected from the combined amino acid substitutions of the following Table 3a.

TABLE 3a
SEQ ID NO: 2 or SEQ ID NO: 4 (combined amino acid substitutions
obtained by site directed mutagenesis.)

| | | Key amino | |
|---|---|---|---|
| Combination | SEQ | acid position | Preferred |
| Number | ID NO: | combination | Substitution |
| 1 | 2 or 4 | Leu397 | Gly |
| | | Phe420 | Met |
| 2 | 2 or 4 | Leu397 | Ala |
| | | Phe420 | Met |
| 3 | 2 or 4 | Leu397 | Ser |
| | | Phe420 | Met |
| 4 | 2 or 4 | Leu397 | Thr |
| | | Phe420 | Met |
| 5 | 2 or 4 | Leu397 | Cys |
| | | Phe420 | Met |
| 6 | 2 or 4 | Leu397 | Val |
| | | Phe420 | Met |
| 7 | 2 or 4 | Leu397 | Ile |
| | | Phe420 | Met |
| 8 | 2 or 4 | Leu397 | Met |
| | | Phe420 | Met |
| 9 | 2 or 4 | Leu397 | Pro |
| | | Phe420 | Met |
| 10 | 2 or 4 | Leu397 | Phe |
| | | Phe420 | Met |
| 11 | 2 or 4 | Leu397 | Tyr |
| | | Phe420 | Met |
| 12 | 2 or 4 | Leu397 | Trp |
| | | Phe420 | Met |
| 13 | 2 or 4 | Leu397 | Asp |
| | | Phe420 | Met |
| 14 | 2 or 4 | Leu397 | Glu |
| | | Phe420 | Met |
| 15 | 2 or 4 | Leu397 | Asn |
| | | Phe420 | Met |
| 16 | 2 or 4 | Leu397 | Gln |
| | | Phe420 | Met |
| 17 | 2 or 4 | Leu397 | His |
| | | Phe420 | Met |
| 18 | 2 or 4 | Leu397 | Lys |
| | | Phe420 | Met |
| 19 | 2 or 4 | Leu397 | Arg |
| | | Phe420 | Met |
| 20 | 2 or 4 | Leu397 | Gly |
| | | Phe420 | Val |
| 21 | 2 or 4 | Leu397 | Ala |
| | | Phe420 | Val |
| 22 | 2 or 4 | Leu397 | Ser |
| | | Phe420 | Val |
| 23 | 2 or 4 | Leu397 | Thr |
| | | Phe420 | Val |
| 24 | 2 or 4 | Leu397 | Cys |
| | | Phe420 | Val |
| 25 | 2 or 4 | Leu397 | Val |
| | | Phe420 | Val |
| 26 | 2 or 4 | Leu397 | Ile |
| | | Phe420 | Val |
| 27 | 2 or 4 | Leu397 | Met |
| | | Phe420 | Val |
| 28 | 2 or 4 | Leu397 | Pro |
| | | Phe420 | Val |
| 29 | 2 or 4 | Leu397 | Phe |
| | | Phe420 | Val |
| 30 | 2 or 4 | Leu397 | Tyr |
| | | Phe420 | Val |
| 31 | 2 or 4 | Leu397 | Trp |
| | | Phe420 | Val |
| 32 | 2 or 4 | Leu397 | Asp |
| | | Phe420 | Val |
| 33 | 2 or 4 | Leu397 | Glu |
| | | Phe420 | Val |
| 34 | 2 or 4 | Leu397 | Asn |
| | | Phe420 | Val |
| 35 | 2 or 4 | Leu397 | Gln |
| | | Phe420 | Val |
| 36 | 2 or 4 | Leu397 | His |
| | | Phe420 | Val |
| 37 | 2 or 4 | Leu397 | Lys |
| | | Phe420 | Val |
| 38 | 2 or 4 | Leu397 | Arg |
| | | Phe420 | Val |

In a further particularly preferred embodiment, the variant or derivative of the mut-PPO of SEQ ID NO: 2 or SEQ ID NO: 4 is selected from the combined amino acid substitutions of the following Table 3b.

TABLE 3b
SEQ ID NO: 2 or SEQ ID NO: 4 (combined
amino acid substitutions)

| | | Key amino | |
|---|---|---|---|
| Combination | SEQ | acid position | Preferred |
| Number | ID NO: | combination | Substitution |
| 39 | 2 or 4 | Leu397 | Asp, Glu, Gln, Asn |
| | | Leu400 | Ala, Ile, Val, Met |
| 40 | 2 or 4 | Leu397 | Asp, Glu, Gln, Asn |
| | | Phe457 | Met, Ala, Leu, Ile, Val |
| 41 | 2 or 4 | Phe204 | Ala, Leu, Ile, Val |
| | | Leu397 | Asp, Glu, Gln, Asn |
| 42 | 2 or 4 | Thr208 | Ser |
| | | Leu400 | Ala, Ile, Val, Met |
| 43 | 2 or 4 | Leu400 | Ala |
| | | Phe457 | Met, Ala, Leu, Ile, Val |
| 44 | 2 or 4 | Phe204 | Ile |
| | | Leu400 | Ala, Ile, Val, Met |
| 45 | 2 or 4 | The208 | Ser |
| | | Phe457 | Met, Ala, Leu, Ile, Val |
| 46 | 2 or 4 | Phe204 | Ile |
| | | Thr208 | Ser |
| 47 | 2 or 4 | Phe204 | Ile |
| | | Phe457 | Met, Ala, Leu, Ile, Val |
| 48 | 2 or 4 | Leu400 | Ala, Ile, Val, Met |
| | | Phe420 | Val, Met, Ala, Ile, Leu |
| 49 | 2 or 4 | Phe204 | Ile |
| | | Phe420 | Val, Met, Ala, Ile, Leu |
| 50 | 2 or 4 | Phe420 | Val, Met, Ala, Ile, Leu |
| | | Phe457 | Met, Ala, Leu, Ile, Val |
| 51 | 2 or 4 | Arg128 | Ala, Leu, Ile, Val |
| | | Leu397 | Asp, Glu, Gln, Asn |
| 52 | 2 or 4 | Thr208 | Ser |
| | | Leu397 | Asp, Glu, Gln, Asn |
| | | Phe457 | Met, Ala, Leu, Ile, Val |
| 53 | 2 or 4 | Phe204 | Ile |
| | | The208 | Ser |
| | | Leu397 | Asp, Glu, Gln, Asn |
| 54 | 2 or 4 | Phe204 | Ile |
| | | Leu397 | Asp, Glu, Gln, Asn |
| | | Phe457 | Met, Ala, Leu, Ile, Val |
| 55 | 2 or 4 | Thr208 | Ser |
| | | Leu400 | Ala, Ile, Val, Met |
| | | Phe457 | Met, Ala, Leu, Ile, Val |
| 56 | 2 or 4 | Phe204 | Ile |
| | | Thr208 | Ser |
| | | Leu400 | Ala, Ile, Val, Met |
| 57 | 2 or 4 | Phe204 | Ile |
| | | Leu400 | Ala, Ile, Val, Met |
| | | Phe457 | Met, Ala, Leu, Ile, Val |
| 58 | 2 or 4 | Thr208 | Ser |
| | | Leu397 | Asp, Glu, Gln, Asn |
| | | Leu400 | Ala, Ile, Val, Met |
| 59 | 2 or 4 | Leu397 | Asp, Glu, Gln, Asn |
| | | Leu400 | Ala, Ile, Val, Met |
| | | Phe457 | Met, Ala, Leu, Ile, Val |
| 60 | 2 or 4 | Phe204 | Ile |
| | | Leu397 | Asp, Glu, Gln, Asn |
| | | Leu400 | Ala, Ile, Val, Met |
| 61 | 2 or 4 | Phe204 | Ile |
| | | Thr208 | Ser |
| | | Phe457 | Met, Ala, Leu, Ile, Val |
| 62 | 2 or 4 | Leu397 | Asp, Glu, Gln, Asn |
| | | Leu400 | Ala, Ile, Val, Met |
| | | Phe420 | Met, Ala, Leu, Ile, Val |
| 63 | 2 or 4 | Leu397 | Asp, Glu, Gln, Asn |
| | | Phe420 | Met, Ala, Leu, Ile, Val |
| | | Phe457 | Met, Ala, Leu, Ile, Val |
| 64 | 2 or 4 | Phe204 | Ile |
| | | Leu397 | Asp, Glu, Gln, Asn |
| | | Phe420 | Met, Ala, Leu, Ile, Val |
| 65 | 2 or 4 | Phe204 | Ile |
| | | Thr208 | Ser |
| | | Phe420 | Met, Ala, Leu, Ile, Val |
| 66 | 2 or 4 | Thr208 | Ser |
| | | Phe420 | Met, Ala, Leu, Ile, Val |
| | | Phe457 | Met, Ala, Leu, Ile, Val |
| 67 | 2 or 4 | Phe204 | Ile |
| | | Phe420 | Met, Ala, Leu, Ile, Val |
| | | Phe457 | Met, Ala, Leu, Ile, Val |
| 68 | 2 or 4 | Arg128 | Ala, Leu, Ile, Val |
| | | Leu400 | Ala, Ile, Val, Met |
| | | Phe420 | Met, Ala, Leu, Ile, Val |
| 69 | 2 or 4 | Arg128 | Ala, Leu, Ile, Val |
| | | Leu397 | Asp, Glu, Gln, Asn |
| | | Phe420 | Met, Ala, Leu, Ile, Val |
| 70 | 2 or 4 | Arg128 | Ala, Leu, Ile, Val |
| | | Phe204 | Ile |
| | | Phe420 | Met, Ala, Leu, Ile, Val |
| 71 | 2 or 4 | Arg128 | Ala, Leu, Ile, Val |
| | | Phe420 | Met, Ala, Leu, Ile, Val |
| | | Phe457 | Met, Ala, Leu, Ile, Val |
| 72 | 2 or 4 | Thr208 | Ser |
| | | Leu400 | Ala, Ile, Val, Met |
| | | Phe420 | Met, Ala, Leu, Ile, Val |
| 73 | 2 or 4 | Leu400 | Ala, Ile, Val, Met |
| | | Phe420 | Met, Ala, Leu, Ile, Val |
| | | Phe457 | Met, Ala, Leu, Ile, Val |
| 74 | 2 or 4 | Phe204 | Ile |
| | | Leu400 | Ala, Ile, Val, Met |
| | | Phe420 | Met, Ala, Leu, Ile, Val |
| 75 | 2 or 4 | Phe204 | Ile |
| | | Thr208 | Se |
| | | Leu400 | Ala, Ile, Val, Met |
| | | Phe457 | Met, Ala, Leu, Ile, Val |
| 76 | 2 or 4 | Phe204 | Ile |
| | | Thr208 | Ser |
| | | Phe420 | Met, Ala, Leu, Ile, Val |
| | | Phe457 | Met, Ala, Leu, Ile, Val |
| 77 | 2 or 4 | Arg128 | Ala, Leu, Ile, Val |
| | | Phe204 | Ile |
| | | Phe420 | Met, Ala, Leu, Ile, Val |
| | | Phe457 | Met, Ala, Leu, Ile, Val |
| 78 | 2 or 4 | Phe204 | Ile |
| | | Leu400 | Ala, Ile, Val, Met |
| | | Phe420 | Met, Ala, Leu, Ile, Val |
| | | Phe457 | Met, Ala, Leu, Ile, Val |
| 79 | 2 or 4 | Phe204 | Ile |
| | | Thr208 | Ser |
| | | Leu397 | Asp, Glu, Gln, Asn |
| | | Leu400 | Ala, Ile, Val, Met |
| 80 | 2 or 4 | Phe204 | Ile |
| | | Thr208 | Ser |
| | | Leu400 | Ala, Ile, Val, Met |
| | | Phe420 | Met, Ala, Leu, Ile, Val |
| | | Phe457 | Met, Ala, Leu, Ile, Val |
| 81 | 2 or 4 | Arg128 | Ala, Leu, Ile, Val |
| | | Phe204 | Ile |
| | | Leu400 | Ala, lle, Val, Met |
| | | Phe420 | Met, Ala, Leu, Ile, Val |
| | | Phe457 | Met, Ala, Leu, Ile, Val |

It is to be understood that any amino acid besides the ones mentioned in the above tables 3 could be used as a substitutent. Assays to test for the functionality of such mutants are readily available in the art, and respectively, described in the Example section of the present invention.

In a preferred embodiment, the amino acid sequence differs from an amino acid sequence of a PPO of SEQ ID NO: 2 or SEQ ID NO: 4 at one or more of the following positions: 128, 204, 208, 397, 400, 420, 457.

Examples of differences at these amino acid positions include, but are not limited to, one or more of the following:

the amino acid at position 128 is other than Arginine;
the amino acid at position 204 is other than Phenylalanine;
the amino acid at position 208 is other than Threonine;
the amino acid at position 397 is other than Leucine,
the amino acid at position 400 is other than Leucine,
the amino acid at position 420 is other than Phenylalanine,
the amino acid at position 457 is other than Phenylalanine.

In some embodiments, the mut-PPO enzyme of SEQ ID NO: 2 or SEQ ID NO: 4 comprises one or more of the following:
the amino acid at position 128 is Leu, Ala, Val, or Ile;
the amino acid at position 204 is Ala, Leu, Ile, or Val;
the amino acid at position 208 is Ser;
the amino acid at position 397 is Gly, Ala, Ser, Thr, Cys, Val, Ile, Met, Pro, Phe, Tyr, Trp, His, Lys, Arg, Asn, Asp, Glu, or Gln;
the amino acid at position 400 is Ala, Ile, Val, or Met;
the amino acid at position 420 is Val, Met, Ala, Ile, or Leu;
the amino acid at position 457 is Met, Ala, Leu, Ile, Val;

In a preferred embodiment, the mut-PPO comprises a sequence of SEQ ID NO: 2 or SEQ ID NO: 4, a variant, derivative, orthologue, paralogue or homologue thereof, in which:
the amino acid at position 420 is Met, and the the amino acid at position 397 is Gly, Ala, Ser, Thr, Cys, Val, Ile, Met, Pro, Phe, Tyr, Trp, His, Lys, Arg, Asn, Asp, Glu, or Gln.

In another preferred embodiment, the mut-PPO comprises a sequence of SEQ ID NO: 2 or SEQ ID NO: 4, a variant, derivative, orthologue, paralogue or homologue thereof, in which:
the amino acid at position 420 is Val, and the the amino acid at position 397 is Gly, Ala, Ser, Thr, Cys, Val, Ile, Met, Pro, Phe, Tyr, Trp, His, Lys, Arg, Asn, Asp, Glu, or Gln.

In another preferred embodiment, the mut-PPO comprises a sequence of SEQ ID NO: 2 or SEQ ID NO: 4, a variant, derivative, orthologue, paralogue or homologue thereof, in which:
the amino acid at position 397 is Asn, Asp, Glu, or Gln, and the amino acid at position 400 is Ala, Ile, Val, or Met.

In another preferred embodiment, the mut-PPO comprises a sequence of SEQ ID NO: 2 or SEQ ID NO: 4, a variant, derivative, orthologue, paralogue or homologue thereof, in which:
the amino acid at position 397 is Asn, Asp, Glu, or Gln, and the amino acid at position 457 is Met, Ala, Leu, Ile, or Val.

In another preferred embodiment, the mut-PPO comprises a sequence of SEQ ID NO: 2 or SEQ ID NO: 4, a variant, derivative, orthologue, paralogue or homologue thereof, in which:
the amino acid at position 204 is Ala, Leu, Ile, or Val, and the amino acid at position 397 is Asn, Asp, Glu, or Gln.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, and the amino acid at position 400 is Ala, Ile, Val, or Met.

In another preferred embodiment, the mut-PPO comprises a sequence of SEQ ID NO: 2 or SEQ ID NO: 4 a variant, derivative, orthologue, paralogue or homologue thereof, in which:
the amino acid at position 400 is Ala, Ile, Val, or Met, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, and the amino acid at position 400 is Ala, Ile, Val, or Met.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, and the amino acid at position 208 is Ser. In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 400 is Ala, Ile, Val, or Met, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, and the amino acid at position 397 is Asn, Asp, Glu, or Gln.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, the amino acid at position 397 is Asn, Asp, Glu, or Gin, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, and the amino acid at position 397 is Asn, Asp, Glu, or Gln.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 397 is Asn, Asp, Glu, or Gin, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, the amino acid at position 400 is Ala, Ile, Val, or Met, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, and the amino acid at position 400 is Ala, Ile, Val, or Met.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 400 is Ala, Ile,
Val, or Met, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, the amino acid at position 397 is Asn, Asp, Glu, or Gln, and the amino acid at position 400 is Ala, Ile, Val, or Met.

In another preferred embodiment, the mut-PPO comprises a sequence of SEQ ID NO: 2 or SEQ ID NO: 4 a variant, derivative, orthologue, paralogue or homologue thereof, in which:
the amino acid at position 397 is Asn, Asp, Glu, or Gln, the amino acid at position 400 is Ala, Ile, Val, or Met, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 397 is Asn, Asp, Glu, or Gln, and the amino acid at position 400 is Ala, Ile, Val, or Met.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 397 is Asn, Asp, Glu, or Gin, the amino acid at position 400 is Ala, Ile,
Val, or Met, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 397 is Asn, Asp, Glu, or Gln, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 397 is Asn, Asp, Glu, or Gin, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, the amino acid at position 400 is Ala, Ile, Val, or Met, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, the amino acid at position 397 is Asn, Asp, Glu, or Gin, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, the amino acid at position 204 is Ala, Leu, Ile, or Val, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 208 is Ser, the amino acid at position 400 is Ala, Ile, Val, or Met, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 400 is Ala, Ile, Val, or Met, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 400 is Ala, Ile, Val, or Met, and the amino acid at position 420 is Val, Met, Ala, Ile, or Leu.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, the amino acid at position 400 is Ala, Ile, Val, or Met, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, the amino acid at position 204 is Ala, Leu, Ile, or Val, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 400 is Ala, Ile, Val, or Met, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, the amino acid at position 397 is Asn, Asp, Glu, or Gin, and the amino acid at position 400 is Ala, Ile, Val, or Met.

In another preferred embodiment, the mut-PPO comprises a sequence of SEQ ID NO: 2 or SEQ ID NO: 4 a variant, derivative, orthologue, paralogue or homologue thereof, in which:
the amino acid at position 204 is Ala, Leu, Ile, or Val, the amino acid at position 208 is Ser, the amino acid at position 400 is Ala, Ile, Val, or Met, the amino acid at position 420 is Val, Met, Ala, Ile, or Leu, and the amino acid at position 457 is Met, Ala, Leu, Ile, or Val.

In another preferred embodiment, the mut-PPO comprises a sequence of SEQ ID NO: 2 or SEQ ID NO: 4 a variant, derivative, orthologue, paralogue or homologue thereof, in which:
the amino acid at position 128 is Leu, Ala, Val, or Ile, the amino acid at position 204 is Ala, Leu, Ile, or Val, the amino acid at position 400 is Ala, Ile, Val, or Met, the amino acid at position 420 is Val, Met, Ala, Ile, or Leu, and the amino acid at position 457 is Met, Ala, Leu, Ile, or Val.

It will be within the knowledge of the skilled artisan to identify conserved regions and motifs shared between the homologues, orthologues and paralogues encoded by SEQ ID NO: 1, 3, 5, 7, 9, 11, 13, 15, 17, 19, 21, 23, 25, 27, 29, 31, 33, 35, 37, 39, 41, 43, or 45, such as those depicted in Table 1. Having identified such conserved regions that may represent suitable binding motifs, amino acids corresponding to the amino acids listed in Table 3a and 3b, can be chosen to be substituted by any other amino acid, preferably by conserved amino acids as shown in table 2, and more preferably by the amino acids of tables 3a and 3b.

In addition, the present invention refers to a method for identifying a benzoxazinone-derivative herbicide by using a mut-PPO encoded by a nucleic acid which comprises the nucleotide sequence of SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof.

Said method comprises the steps of:
a) generating a transgenic cell or plant comprising a nucleic acid encoding a mut-PPO, wherein the mut-PPO is expressed;
b) applying a benzoxazinone-derivative herbicide to the transgenic cell or plant of a) and to a control cell or plant of the same variety;
c) determining the growth or the viability of the transgenic cell or plant and the control cell or plant after application of said benzoxazinone-derivative herbicide, and
d) selecting “benzoxazinone-derivative herbicides” which confer reduced growth to the control cell or plant as compared to the growth of the transgenic cell or plant.

By “control cell” or “similar, wild-type, plant, plant tissue, plant cell or host cell” is intended a plant, plant tissue, plant cell, or host cell, respectively, that lacks the herbicide-resistance characteristics and/or particular polynucleotide of the invention that are disclosed herein. The use of the term “wild-type” is not, therefore, intended to imply that a plant, plant tissue, plant cell, or other host cell lacks recombinant DNA in its genome, and/or does not possess herbicide-resistant characteristics that are different from those disclosed herein.

Another object refers to a method of identifying a nucleotide sequence encoding a mut-PPO which is resistant or tolerant to a benzoxazinone-derivative herbicide, the method comprising:
a) generating a library of mut-PPO-encoding nucleic acids,
b) screening a population of the resulting mut-PPO-encoding nucleic acids by expressing each of said nucleic acids in a cell or plant and treating said cell or plant with a benzoxazinone-derivative herbicide,
c) comparing the benzoxazinone-derivative herbicide-tolerance levels provided by said population of mut-PPO encoding nucleic acids with the benzoxazinone-derivative herbicide-tolerance level provided by a control PPO-encoding nucleic acid,
d) selecting at least one mut-PPO-encoding nucleic acid that provides a significantly increased level of tolerance to a benzoxazinone-derivative herbicide as compared to that provided by the control PPO-encoding nucleic acid.

In a preferred embodiment, the mut-PPO-encoding nucleic acid selected in step d) provides at least 2-fold as much resistance or tolerance of a cell or plant to a benzoxazinone-derivative herbicide as compared to that provided by the control PPO-encoding nucleic acid.

In a further preferred embodiment, the mut-PPO-encoding nucleic acid selected in step d) provides at least 2-fold, at least 5-fold, at least 10-fold, at least 20-fold, at least 50-fold, at least 100-fold, at least 500-fold, as much resistance or tolerance of a cell or plant to a benzoxazinone-derivative herbicide as compared to that provided by the control PPO-encoding nucleic acid.

The resistance or tolerance can be determined by generating a transgenic plant or host cell, preferably a plant cell, comprising a nucleic acid sequence of the library of step a) and comparing said transgenic plant with a control plant or host cell, preferably a plant cell.

Another object refers to a method of identifying a plant or algae containing a nucleic acid comprising a nucleotide sequence encoding a wild-type or mut-PPO which is resistant or tolerant to a benzoxazinone-derivative herbicide, the method comprising:
a) identifying an effective amount of a benzoxazinone-derivative herbicide in a culture of plant cells or green algae that leads to death of said cells.
b) treating said plant cells or green algae with a mutagenizing agent,
c) contacting said mutagenized cells population with an effective amount of benzoxazinone-derivative herbicide, identified in a),
d) selecting at least one cell surviving these test conditions,
e) PCR-amplification and sequencing of PPO genes from cells selected in d) and comparing such sequences to wild-type PPO gene sequences, respectively.

In a preferred embodiment, said mutagenizing agent is ethylmethanesulfonate (EMS).

Many methods well known to the skilled artisan are available for obtaining suitable candidate nucleic acids for identifying a nucleotide sequence encoding a mut-PPO from a variety of different potential source organisms including microbes, plants, fungi, algae, mixed cultures etc. as well as environmental sources of DNA such as soil. These methods include inter alia the preparation of cDNA or genomic DNA libraries, the use of suitably degenerate oligonucleotide primers, the use of probes based upon known sequences or complementation assays (for example, for growth upon tyrosine) as well as the use of mutagenesis and shuffling in order to provide recombined or shuffled mut-PPO-encoding sequences.

Nucleic acids comprising candidate and control PPO encoding sequences can be expressed in yeast, in a bacterial host strain, in an alga or in a higher plant such as tobacco or Arabidopsis and the relative levels of inherent tolerance of the PPO encoding sequences screened according to a visible indicator phenotype of the transformed strain or plant in the presence of different concentrations of the selected benzoxazinone-derivative herbicide. Dose responses and relative shifts in dose responses associated with these indicator phenotypes (formation of brown color, growth inhibition, herbicidal effect etc) are conveniently expressed in terms, for example, of GR50 (concentration for 50% reduction of growth) or MIC (minimum inhibitory concentration) values where increases in values correspond to increases in inherent tolerance of the expressed PPO. For example, in a relatively rapid assay system based upon transformation of a bacterium such as E. coli, each mut-PPO encoding sequence may be expressed, for example, as a DNA sequence under expression control of a controllable promoter such as the lacZ promoter and taking suitable account, for example by the use of synthetic DNA, of such issues as codon usage in order to obtain as comparable a level of expression as possible of different PPO sequences. Such strains expressing nucleic acids comprising alternative candidate PPO sequences may be plated out on different concentrations of the selected benzoxazinone-derivative herbicide in, optionally, a tyrosine supplemented medium and the relative levels of inherent tolerance of the expressed PPO enzymes estimated on the basis of the extent and MIC for inhibition of the formation of the brown, ochronotic pigment.

In another embodiment, candidate nucleic acids are transformed into plant material to generate a transgenic plant, regenerated into morphologically normal fertile plants which are then measured for differential tolerance to selected benzoxazinone-derivative herbicides. Many suitable methods for transformation using suitable selection markers such as kanamycin, binary vectors such as from Agrobacterium and plant regeneration as, for example, from tobacco leaf discs are well known in the art. Optionally, a control population of plants is likewise transformed with a nucleic acid expressing the control PPO. Alternatively, an untransformed dicot plant such as Arabidopsis or Tobacco can be used as a control since this, in any case, expresses its own endogenous PPO. The average, and distribution, of herbicide tolerance levels of a range of primary plant transformation events or their progeny to benzoxazinone-derivative described supra are evaluated in the normal manner based upon plant damage, meristematic bleaching symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent tolerance of the expressed PPO. Herbicides can suitably be applied pre-emergence or post-emergence.

Another object refers to an isolated nucleic acid encoding a mut-PPO, wherein the nucleic acid is identifiable by a method as defined above.

In another embodiment, the invention refers to a plant cell transformed by a wild-type or a mut-PPO nucleic acid or or a plant cell which has been mutated to obtain a plant expressing a wild-type or a mut-PPO nucleic acid, wherein expression of the nucleic acid in the plant cell results in increased resistance or tolerance to a benzoxazinone-derivative herbicide as compared to a wild type variety of the plant cell.

The term “expression/expressing” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

To obtain the desired effect, i.e. plants that are tolerant or resistant to the benzoxazinone-derivative herbicide derivative herbicide of the present invention, it will be understood that the at least one nucleic acid is “over-expressed” by methods and means known to the person skilled in the art.

The term “increased expression” or “overexpression” as used herein means any form of expression that is additional to the original wild-type expression level. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8:4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994)

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8:363-373); electroporation of protoplasts (Shillito R. D. et al. (1985) Bio/Technol 3, 1099-1102); microinjection into plant material (Crossway A et al., (1986) Mol. Gen Genet 202:179-185); DNA or RNA-coated particle bombardment (Klein T M et al., (1987) Nature 327:70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199:612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N—H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5:551-558; Katavic (1994). Mol Gen Genet, 245:363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316:1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3): 425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229). The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Preferably, the wild-type or mut-PPO nucleic acid comprises a polynucleotide sequence selected from the group consisting of: a) a polynucleotide as shown in SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof; b) a polynucleotide encoding a polypeptide as shown in SEQ ID NO: 2, 4, 6, 8, 10, 12, 14, 16, 18, 20, 22, 24, 26, 28, 30, 32, 34, 36, 38, 40, 42, 44, or 46, or a variant or derivative thereof; c) a polynucleotide comprising at least 60 consecutive nucleotides of any of a) or b); and d) a polynucleotide complementary to the polynucleotide of any of a) through c).

Preferably, the expression of the nucleic acid in the plant results in the plant's increased resistance to benzoxazinone-derivative herbicide as compared to a wild type variety of the plant.

In another embodiment, the invention refers to a plant, preferably a transgenic plant, comprising a plant cell according to the present invention, wherein expression of the nucleic acid in the plant results in the plant's increased resistance to benzoxazinone-derivative herbicide as compared to a wild type variety of the plant.

The plants described herein can be either transgenic crop plants or non-transgenic plants.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein. Furthermore, the term “transgenic” refers to any plant, plant cell, callus, plant tissue, or plant part, that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

Plants containing mutations arising due to non-spontaneous mutagenesis and selective breeding are referred to herein as non-transgenic plants and are included in the present invention. In embodiments wherein the plant is transgenic and comprises multiple mut-PPO nucleic acids, the nucleic acids can be derived from different genomes or from the same genome. Alternatively, in embodiments wherein the plant is non-transgenic and comprises multiple mut-PPO nucleic acids, the nucleic acids are located on different genomes or on the same genome.

In certain embodiments, the present invention involves herbidicide-resistant plants that are produced by mutation breeding. Such plants comprise a polynucleotide encoding a mut-PPO and are tolerant to one or more “benzoxazinone-derivative herbicides”. Such methods can involve, for example, exposing the plants or seeds to a mutagen, particularly a chemical mutagen such as, for example, ethyl methanesulfonate (EMS) and selecting for plants that have enhanced tolerance to at least one or more benzoxazinone-derivative herbicide.

However, the present invention is not limited to herbicide-tolerant plants that are produced by a mutagenesis method involving the chemical mutagen EMS. Any mutagenesis method known in the art may be used to produce the herbicide-resistant plants of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Herbicide-resistant plants can also be produced by using tissue culture methods to select for plant cells comprising herbicide-resistance mutations and then regenerating herbicide-resistant plants therefrom. See, for example, U.S. Pat. Nos. 5,773,702 and 5,859,348, both of which are herein incorporated in their entirety by reference. Further details of mutation breeding can be found in “Principals of Cultivar Development” Fehr, 1993 Macmillan Publishing Company the disclosure of which is incorporated herein by reference

In addition to the definition above, the term “plant” is intended to encompass crop plants at any stage of maturity or development, as well as any tissues or organs (plant parts) taken or derived from any such plant unless otherwise clearly indicated by context. Plant parts include, but are not limited to, stems, roots, flowers, ovules, stamens, leaves, embryos, meristematic regions, callus tissue, anther cultures, gametophytes, sporophytes, pollen, microspores, protoplasts, and the like.

The plant of the present invention comprises at least one mut-PPO nucleic acid or over-expressed wild-type PPO nucleic acid, and has increased tolerance to a benzoxazinone-derivative herbicide as compared to a wild-type variety of the plant. It is possible for the plants of the present invention to have multiple wild-type or mut-PPO nucleic acids from different genomes since these plants can contain more than one genome. For example, a plant contains two genomes, usually referred to as the A and B genomes. Because PPO is a required metabolic enzyme, it is assumed that each genome has at least one gene coding for the PPO enzyme (i.e. at least one PPO gene). As used herein, the term “PPO gene locus” refers to the position of an PPO gene on a genome, and the terms “PPO gene” and “PPO nucleic acid” refer to a nucleic acid encoding the PPO enzyme. The PPO nucleic acid on each genome differs in its nucleotide sequence from an PPO nucleic acid on another genome. One of skill in the art can determine the genome of origin of each PPO nucleic acid through genetic crossing and/or either sequencing methods or exonuclease digestion methods known to those of skill in the art.

The present invention includes plants comprising one, two, three, or more mut-PPO alleles, wherein the plant has increased tolerance to a benzoxazinone-derivative herbicide as compared to a wild-type variety of the plant. The mut-PPO alleles can comprise a nucleotide sequence selected from the group consisting of a polynucleotide as defined in SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof, a polynucleotide encoding a polypeptide as defined in SEQ ID NO: 2, 4, 6, 8, 10, 12, 14, 16, 18, 20, 22, 24, 26, 28, 30, 32, 34, 36, 38, 40, 42, 44, or 46, or a variant or derivative, homologue, orthologue, paralogue thereof, a polynucleotide comprising at least 60 consecutive nucleotides of any of the aforementioned polynucleotides; and a polynucleotide complementary to any of the aforementioned polynucleotides.

“Alleles” or “allelic variants” are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms

The term “variety” refers to a group of plants within a species defined by the sharing of a common set of characteristics or traits accepted by those skilled in the art as sufficient to distinguish one cultivar or variety from another cultivar or variety. There is no implication in either term that all plants of any given cultivar or variety will be genetically identical at either the whole gene or molecular level or that any given plant will be homozygous at all loci. A cultivar or variety is considered “true breeding” for a particular trait if, when the true-breeding cultivar or variety is self-pollinated, all of the progeny contain the trait. The terms “breeding line” or “line” refer to a group of plants within a cultivar defined by the sharing of a common set of characteristics or traits accepted by those skilled in the art as sufficient to distinguish one breeding line or line from another breeding line or line. There is no implication in either term that all plants of any given breeding line or line will be genetically identical at either the whole gene or molecular level or that any given plant will be homozygous at all loci. A breeding line or line is considered “true breeding” for a particular trait if, when the true-breeding line or breeding line is self-pollinated, all of the progeny contain the trait. In the present invention, the trait arises from a mutation in a PPO gene of the plant or seed.

The herbicide-resistant plants of the invention that comprise polynucleotides encoding mut-PPO polypeptides also find use in methods for increasing the herbicide-resistance of a plant through conventional plant breeding involving sexual reproduction. The methods comprise crossing a first plant that is a herbicide-resistant plant of the invention to a second plant that may or may not be resistant to the same herbicide or herbicides as the first plant or may be resistant to different herbicide or herbicides than the first plant. The second plant can be any plant that is capable of producing viable progeny plants (i.e., seeds) when crossed with the first plant. Typically, but not necessarily, the first and second plants are of the same species. The methods can optionally involve selecting for progeny plants that comprise the mut-PPO polypeptides of the first plant and the herbicide resistance characteristics of the second plant. The progeny plants produced by this method of the present invention have increased resistance to a herbicide when compared to either the first or second plant or both. When the first and second plants are resistant to different herbicides, the progeny plants will have the combined herbicide tolerance characteristics of the first and second plants. The methods of the invention can further involve one or more generations of backcrossing the progeny plants of the first cross to a plant of the same line or genotype as either the first or second plant. Alternatively, the progeny of the first cross or any subsequent cross can be crossed to a third plant that is of a different line or genotype than either the first or second plant. The present invention also provides plants, plant organs, plant tissues, plant cells, seeds, and non-human host cells that are transformed with the at least one polynucleotide molecule, expression cassette, or transformation vector of the invention. Such transformed plants, plant organs, plant tissues, plant cells, seeds, and non-human host cells have enhanced tolerance or resistance to at least one herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana and crop plants.

It is to be understood that the plant of the present invention can comprise a wild type PPO nucleic acid in addition to a mut-PPO nucleic acid. It is contemplated that the benzoxazinone-derivative herbicide tolerant lines may contain a mutation in only one of multiple PPO isoenzymes. Therefore, the present invention includes a plant comprising one or more mut-PPO nucleic acids in addition to one or more wild type PPO nucleic acids.

In another embodiment, the invention refers to a seed produced by a transgenic plant comprising a plant cell of the present invention, wherein the seed is true breeding for an increased resistance to a benzoxazinone-derivative herbicide as compared to a wild type variety of the seed.

In another embodiment, the invention refers to a method of producing a transgenic plant cell with an increased resistance to a benzoxazinone-derivative herbicide as compared to a wild type variety of the plant cell comprising, transforming the plant cell with an expression cassette comprising a mut-PPO nucleic acid.

In another embodiment, the invention refers to a method of producing a transgenic plant comprising, (a) transforming a plant cell with an expression cassette comprising a mut-PPO nucleic acid, and (b) generating a plant with an increased resistance to benzoxazinone-derivative herbicide from the plant cell.

Consequently, mut-PPO nucleic acids of the invention are provided in expression cassettes for expression in the plant of interest. The cassette will include regulatory sequences operably linked to a mut-PPO nucleic acid sequence of the invention. The term “regulatory element” as used herein refers to a polynucleotide that is capable of regulating the transcription of an operably linked polynucleotide. It includes, but not limited to, promoters, enhancers, introns, 5′ UTRs, and 3′ UTRs. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the mut-PPO nucleic acid sequence to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a mut-PPO nucleic acid sequence of the invention, and a transcriptional and translational termination region (i.e., termination region) functional in plants. The promoter may be native or analogous, or foreign or heterologous, to the plant host and/or to the mut-PPO nucleic acid sequence of the invention. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the mut-PPO nucleic acid sequence of the invention, it is intended that the promoter is not the native or naturally occurring promoter for the operably linked mut-PPO nucleic acid sequence of the invention. As used herein, a chimeric gene comprises a coding sequence operably linked to a transcription initiation region that is heterologous to the coding sequence.

While it may be preferable to express the mut-PPO nucleic acids of the invention using heterologous promoters, the native promoter sequences may be used. Such constructs would change expression levels of the mut-PPO protein in the plant or plant cell. Thus, the phenotype of the plant or plant cell is altered.

The termination region may be native with the transcriptional initiation region, may be native with the operably linked mut-PPO sequence of interest, may be native with the plant host, or may be derived from another source (i.e., foreign or heterologous to the promoter, the mut-PPO nucleic acid sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas t al. (1989) Nucleic Acids Res. 17:7891-7903; and Joshi et al. (1987) Nucleic Acid Res. 15:9627-9639. Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference. Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures. Nucleotide sequences for enhancing gene expression can also be used in the plant expression vectors. These include the introns of the maize Adhl, intronl gene (Callis et al. Genes and Development 1:1183-1200, 1987), and leader sequences, (W-sequence) from the Tobacco Mosaic virus (TMV), Maize Chlorotic Mottle Virus and Alfalfa Mosaic Virus (Gallie et al. Nucleic Acid Res. 15:8693-8711, 1987 and Skuzeski et al. Plant Mol. Biol. 15:65-79, 1990). The first intron from the shrunken-1 locus of maize, has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize mut-PPO gene expression, the plant expression vectors of the invention may also contain DNA sequences containing matrix attachment regions (MARs). Plant cells transformed with such modified expression systems, then, may exhibit overexpression or constitutive expression of a nucleotide sequence of the invention.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. ScL USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and trans versions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced mut-PPO expression within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka e/[alpha]/. (1993) Proc Natl. Acad. Sci. USA 90(20):9586-9590; and Guevara-Garcia et al. (1993) Plant J. 4(3):495-505. Such promoters can be modified, if necessary, for weak expression. In one embodiment, the nucleic acids of interest are targeted to the chloroplast for expression. In this manner, where the nucleic acid of interest is not directly inserted into the chloroplast, the expression cassette will additionally contain a chloroplast-targeting sequence comprising a nucleotide sequence that encodes a chloroplast transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the mut-PPO nucleic acid of the invention such that the two sequences are contiguous and in the same reading frame. See, for example, Von Heijne et al. (1991) Plant Mol. Biol. Rep. 9: 104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. While the mut-PPO proteins of the invention include a native chloroplast transit peptide, any chloroplast transit peptide known in the art can be fused to the amino acid sequence of a mature mut-PPO protein of the invention by operably linking a choloroplast-targeting sequence to the 5′-end of a nucleotide sequence encoding a mature mut-PPO protein of the invention. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl) shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263: 14996-14999). See also Von Heijne et al. (1991) Plant Mol. Biol. Rep. 9: 104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196: 1414-1421; and Shah et al. (1986) Science 233:478-481.

Methods for transformation of chloroplasts are known in the art. See, for example, Svab et al. (1990) Proc. Natl. Acad. ScL USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305. The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

In a preferred embodiment, the mut-PPO nucleic acid comprises a polynucleotide sequence selected from the group consisting of: a) a polynucleotide as shown in SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof; b) a polynucleotide encoding a polypeptide as shown in SEQ ID NO: 2, 4, 6, 8, 10, 12, 14, 16, 18, 20, 22, 24, 26, 28, 30, 32, 34, 36, 38, 40, 42, 44, or 46, or a variant or derivative thereof; c) a polynucleotide comprising at least 60 consecutive nucleotides of any of a) or c); and d) a polynucleotide complementary to the polynucleotide of any of a) through c)

Preferably, the expression cassette further comprises a transcription initiation regulatory region and a translation initiation regulatory region that are functional in the plant.

While the polynucleotides of the invention find use as selectable marker genes for plant transformation, the expression cassettes of the invention can include another selectable marker gene for the selection of transformed cells. Selectable marker genes, including those of the present invention, are utilized for the selection of transformed cells or tissues. Marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christophers on et al (1992) Proc. Natl. Acad. ScL USA 89:6314-6318; Yao et al. (1992) Cell 71:63-72; Reznikoff (1992) Mol Microbiol 6:2419-2422; Barkley et al (1980) in The Operon, pp. 177-220; Hu et al (1987) Cell 48:555-566; Brown et al (1987) Cell 49:603-612; Figge et al (1988) Cell 52:713-722; Deuschle et al (1989) Proc. Natl Acad. AcL USA 86:5400-5404; Fuerst et al (1989) Proc. Natl Acad. ScL USA 86:2549-2553; Deuschle et al (1990) Science 248:480-483; Gossen (1993) Ph.D. Thesis, University of Heidelberg; Reines et al (1993) Proc. Natl Acad. ScL USA 90:1917-1921; Labow et al (1990) Mol Cell Biol 10:3343-3356; Zambretti et al (1992) Proc. Natl Acad. ScL USA 89:3952-3956; Bairn et al (1991) Proc. Natl Acad. ScL USA 88:5072-5076; Wyborski et al (1991) Nucleic Acids Res. 19:4647-4653; Hillenand-Wissman (1989) Topics Mol Struc. Biol 10:143-162; Degenkolb et al (1991) Antimicrob. Agents Chemother. 35:1591-1595; Kleinschmidt et al (1988) Biochemistry 27:1094-1104; Bonin (1993) Ph.D. Thesis, University of Heidelberg; Gossen et al (1992) Proc. Natl Acad. ScL USA 89:5547-5551; Oliva et al (1992) Antimicrob. Agents Chemother. 36:913-919; Hlavka et al (1985) Handbook of Experimental Pharmacology, Vol. 78 (Springer-Verlag, Berlin); Gill et al (1988) Nature 334:721-724. Such disclosures are herein incorporated by reference. The above list of selectable marker genes is not meant to be limiting. Any selectable marker gene can be used in the present invention.

The invention further provides an isolated recombinant expression vector comprising the expression cassette containing a mut-PPO nucleic acid as described above, wherein expression of the vector in a host cell results in increased tolerance to a benzoxazinone-derivative herbicide as compared to a wild type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., mut-PPO polypeptides, fusion polypeptides, etc.).

In a preferred embodiment of the present invention, the mut-PPO polypeptides are expressed in plants and plants cells such as unicellular plant cells (such as algae) (See Falciatore et al., 1999, Marine Biotechnology 1(3): 239-251 and references therein) and plant cells from higher plants (e.g., the spermatophytes, such as crop plants). A mut-PPO polynucleotide may be “introduced” into a plant cell by any means, including transfection, transformation or transduction, electroporation, particle bombardment, agroinfection, biolistics, and the like.

Suitable methods for transforming or transfecting host cells including plant cells can be found in Sambrook et al. (Molecular Cloning: A Laboratory Manual. 2nd, ed., Cold Spring Harbor Laboratory, Cold Spring Harbor Laboratory Press, Cold Spring Harbor, NY, 1989) and other laboratory manuals such as Methods in Molecular Biology, 1995, Vol. 44, Agrobacterium protocols, ed: Gartland and Davey, Humana Press, Totowa, New Jersey. As increased tolerance to benzoxazinone-derivative herbicides is a general trait wished to be inherited into a wide variety of plants like maize, wheat, rye, oat, triticale, rice, barley, soybean, peanut, cotton, rapeseed and canola, manihot, pepper, sunflower and tagetes, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. In a preferred embodiment, the plant is a crop plant. Forage crops include, but are not limited to, Wheatgrass, Canarygrass, Bromegrass, Wildrye Grass, Bluegrass, Orchardgrass, Alfalfa, Salfoin, Birdsfoot Trefoil, Alsike Clover, Red Clover, and Sweet Clover.

In one embodiment of the present invention, transfection of a mut-PPO polynucleotide into a plant is achieved by Agrobacterium mediated gene transfer. One transformation method known to those of skill in the art is the dipping of a flowering plant into an Agrobacteria solution, wherein the Agrobacteria contains the mut-PPO nucleic acid, followed by breeding of the transformed gametes. Agrobacterium mediated plant transformation can be performed using for example the GV3101(pMP90) (Koncz and Schell, 1986, Mol. Gen. Genet. 204:383-396) or LBA4404 (Clontech) Agrobacterium tumefaciens strain. Transformation can be performed by standard transformation and regeneration techniques (Deblaere et al., 1994, Nucl. Acids. Res. 13:4777-4788; Gelvin, Stanton B. and Schilperoort, Robert A, Plant Molecular Biology Manual, 2nd Ed.—Dordrecht: Kluwer Academic Publ., 1995.—in Sect., Ringbuc Zentrale Signatur: BT11-P ISBN 0-7923-2731-4; Glick, Bernard R. and Thompson, John E., Methods in Plant Molecular Biology and Biotechnology, Boca Raton: CRC Press, 1993 360 S., ISBN 0-8493-5164-2). For example, rapeseed can be transformed via cotyledon or hypocotyl transformation (Moloney et al., 1989, Plant Cell Report 8:238-242; De Block et al., 1989, Plant Physiol. 91:694-701). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., 1994, Plant Cell Report 13:282-285. Additionally, transformation of soybean can be performed using for example a technique described in European Patent No. 0424 047, U.S. Pat. No. 5,322,783, European Patent No. 0397 687, U.S. Pat. No. 5,376,543, or 5,169,770. Transformation of maize can be achieved by particle bombardment, polyethylene glycol mediated DNA uptake, or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387, and a specific example of wheat transformation can be found in PCT Application No. WO 93/07256.

According to the present invention, the introduced mut-PPO polynucleotide may be maintained in the plant cell stably if it is incorporated into a non-chromosomal autonomous replicon or integrated into the plant chromosomes. Alternatively, the introduced mut-PPO polynucleotide may be present on an extra-chromosomal non-replicating vector and be transiently expressed or transiently active. In one embodiment, a homologous recombinant microorganism can be created wherein the mut-PPO polynucleotide is integrated into a chromosome, a vector is prepared which contains at least a portion of an PPO gene into which a deletion, addition, or substitution has been introduced to thereby alter, e.g., functionally disrupt, the endogenous PPO gene and to create a mut-PPO gene. To create a point mutation via homologous recombination, DNA-RNA hybrids can be used in a technique known as chimeraplasty (Cole-Strauss et al., 1999, Nucleic Acids Research 27(5): 1323-1330 and Kmiec, 1999, Gene therapy American Scientist 87(3): 240-247). Other homologous recombination procedures in Triticum species are also well known in the art and are contemplated for use herein.

In the homologous recombination vector, the mut-PPO gene can be flanked at its 5′ and 3′ ends by an additional nucleic acid molecule of the PPO gene to allow for homologous recombination to occur between the exogenous mut-PPO gene carried by the vector and an endogenous PPO gene, in a microorganism or plant. The additional flanking PPO nucleic acid molecule is of sufficient length for successful homologous recombination with the endogenous gene. Typically, several hundreds of base pairs up to kilobases of flanking DNA (both at the 5′ and 3′ ends) are included in the vector (see e.g., Thomas, K. R., and Capecchi, M. R., 1987, Cell 51:503 for a description of homologous recombination vectors or Strepp et al., 1998, PNAS, 95(8):4368-4373 for cDNA based recombination in Physcomitrella patens). However, since the mut-PPO gene normally differs from the PPO gene at very few amino acids, a flanking sequence is not always necessary. The homologous recombination vector is introduced into a microorganism or plant cell (e.g., via polyethylene glycol mediated DNA), and cells in which the introduced mut-PPO gene has homologously recombined with the endogenous PPO gene are selected using art-known techniques.

In another embodiment, recombinant microorganisms can be produced that contain selected systems that allow for regulated expression of the introduced gene. For example, inclusion of a mut-PPO gene on a vector placing it under control of the lac operon permits expression of the mut-PPO gene only in the presence of IPTG. Such regulatory systems are well known in the art.

Another aspect of the invention pertains to host cells into which a recombinant expression vector of the invention has been introduced. The terms “host cell” and “recombinant host cell” are used interchangeably herein. It is understood that such terms refer not only to the particular subject cell but they also apply to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a mut-PPO polynucleotide can be expressed in bacterial cells such as C. glutamicum, insect cells, fungal cells, or mammalian cells (such as Chinese hamster ovary cells (CHO) or COS cells), algae, ciliates, plant cells, fungi or other microorganisms like C. glutamicum. Other suitable host cells are known to those skilled in the art.

A host cell of the invention, such as a prokaryotic or eukaryotic host cell in culture, can be used to produce (i.e., express) a mut-PPO polynucleotide. Accordingly, the invention further provides methods for producing mut-PPO polypeptides using the host cells of the invention. In one embodiment, the method comprises culturing the host cell of invention (into which a recombinant expression vector encoding a mut-PPO polypeptide has been introduced, or into which genome has been introduced a gene encoding a wild-type or mut-PPO polypeptide) in a suitable medium until mut-PPO polypeptide is produced. In another embodiment, the method further comprises isolating mut-PPO polypeptides from the medium or the host cell. Another aspect of the invention pertains to isolated mut-PPO polypeptides, and biologically active portions thereof. An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations of mut-PPO polypeptide in which the polypeptide is separated from some of the cellular components of the cells in which it is naturally or recombinantly produced. In one embodiment, the language “substantially free of cellular material” includes preparations of a mut-PPO polypeptide having less than about 30% (by dry weight) of non-mut-PPO material (also referred to herein as a “contaminating polypeptide”), more preferably less than about 20% of non-mut-PPO material, still more preferably less than about 10% of non-mut-PPO material, and most preferably less than about 5% non-mut-PPO material.

When the mut-PPO polypeptide, or biologically active portion thereof, is recombinantly produced, it is also preferably substantially free of culture medium, i.e., culture medium represents less than about 20%, more preferably less than about 10%, and most preferably less than about 5% of the volume of the polypeptide preparation. The language “substantially free of chemical precursors or other chemicals” includes preparations of mut-PPO polypeptide in which the polypeptide is separated from chemical precursors or other chemicals that are involved in the synthesis of the polypeptide. In one embodiment, the language “substantially free of chemical precursors or other chemicals” includes preparations of a mut-PPO polypeptide having less than about 30% (by dry weight) of chemical precursors or non-mut-PPO chemicals, more preferably less than about 20% chemical precursors or non-mut-PPO chemicals, still more preferably less than about 10% chemical precursors or non-mut-PPO chemicals, and most preferably less than about 5% chemical precursors or non-mut-PPO chemicals. In preferred embodiments, isolated polypeptides, or biologically active portions thereof, lack contaminating polypeptides from the same organism from which the mut-PPO polypeptide is derived. Typically, such polypeptides are produced by recombinant expression of, for example, a mut-PPO polypeptide in plants other than, or in microorganisms such as C. glutamicum, ciliates, algae, or fungi.

As described above, the present invention teaches compositions and methods for increasing the benzoxazinone-derivative tolerance of a crop plant or seed as compared to a wild-type variety of the plant or seed. In a preferred embodiment, the benzoxazinone-derivative tolerance of a crop plant or seed is increased such that the plant or seed can withstand a benzoxazinone-derivative herbicide application of preferably approximately 1-1000 g ai ha⁻¹, more preferably 20-160 g ai ha⁻¹, and most preferably 40-80 g ai ha⁻¹. As used herein, to “withstand” a benzoxazinone-derivative herbicide application means that the plant is either not killed or not injured by such application.

Furthermore, the present invention provides methods that involve the use of at least one benzoxazinone-derivative herbicide as described in detail supra.

In these methods, the benzoxazinone-derivative herbicide can be applied by any method known in the art including, but not limited to, seed treatment, soil treatment, and foliar treatment. Prior to application, the benzoxazinone-derivative herbicide can be converted into the customary formulations, for example solutions, emulsions, suspensions, dusts, powders, pastes and granules. The use form depends on the particular intended purpose; in each case, it should ensure a fine and even distribution of the compound according to the invention.

By providing plants having increased tolerance to benzoxazinone-derivative herbicide, a wide variety of formulations can be employed for protecting plants from weeds, so as to enhance plant growth and reduce competition for nutrients. A benzoxazinone-derivative herbicide can be used by itself for pre-emergence, post-emergence, pre-planting, and at-planting control of weeds in areas surrounding the crop plants described herein, or a benzoxazinone-derivative herbicide formulation can be used that contains other additives. The benzoxazinone-derivative herbicide can also be used as a seed treatment. Additives found in a benzoxazinone-derivative herbicide formulation include other herbicides, detergents, adjuvants, spreading agents, sticking agents, stabilizing agents, or the like. The benzoxazinone-derivative herbicide formulation can be a wet or dry preparation and can include, but is not limited to, flowable powders, emulsifiable concentrates, and liquid concentrates. The benzoxazinone-derivative herbicide and herbicide formulations can be applied in accordance with conventional methods, for example, by spraying, irrigation, dusting, or the like.

Suitable formulations are describe in detail in PCT/EP2009/063387 and PCT/EP2009/063386, which are incorporated herein by reference.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1: Site-Directed Mutagenesis of Amaranthus PPO

### Cloning of Aramanthus PPO

The Amaranthus tuberculatus coding sequence for PPO-susceptible and -resistant isoforms, and all mutant combinations and multiple mutations, (SEQ ID No: 1, 3, 5, 7) were synthesized and cloned by Geneart (Geneart AG, Regensburg, Germany).

Plasmids were isolated from E. coli TOP10 by performing a plasmid minpreparation and confirmed by DNA sequencing.

### Expression and Purification of Recombinant Wildtype and Mutant PPO

(Taken from: Franck E. Dayan, Pankaj R. Daga, Stephen O. Duke, Ryan M. Lee, Patrick J. Tranel, Robert J. Doerksen. Biochemical and structural consequences of a glycine deletion in the α-8 helix of protoporphyrinogen oxidase. Biochimica et Biophysica Acta 1804 (2010), 1548-56)

Clones in pRSET vector were transformed into BL21 (DE3)-pLysS strain of E. coli. Cells were grown in 250 mL of LB with 100 μgmL-1 of carbenicillin, shaking overnight at 37° C. Cultures were diluted in 1 L of LB with antibiotic and grown at 37° C. shaking for 2 h, induced with 1 mM IPTG and grown at 25° C. shaking for 5 more hours. The cells were harvested by centrifugation at 1600×g, washed with 0.09% NaCl, and stored at −80° C.

Cells were lysed using a French press at 140 MPa in 50 mM sodium phosphate pH 7.5, 1 M NaCl, 5 mM imidazole, 5% glycerol, and 1 μg mL-1 leupeptin. Following lysis, 0.5 U of benzonase (Novagen, EMD Chemicals, Inc., Gibbstown, NJ) and PMSF (final concentration of 1 mM) were added. Cell debris was removed by centrifugation at 3000×g. His-tagged PPO proteins were purified on a nickel activated Hitrap Chelating HP column (GE Healthcare Bio-Sciences Corp., Piscataway, NJ) equilibrated with 20 mM sodium phosphate pH 8.0, 50 mM NaCl, 5 mM imidazole, 5 mM MgCl2, 0.1 mM EDTA, and 17% glycerol.

PPO eluted with 250 mM imidazole. The active protein was desalted on a PD-10 column (GE Healthcare Bio-Sciences Corp., Piscataway, NJ) equilibrated with a 20 mM sodium phosphate buffer, pH 7.5, 5 mM MgCl2, 1 mM EDTA and 17% glycerol. Each liter of culture provided approximately 10 mg of pure PPO, which was stored at −20° C. until being used in assays.

### PPO Activity Assay

PPO Enzyme Assay (non-recombinant). PPO protein (EC 1.3.3.4) was extracted from coleoptiles or shoots (150 g fresh weight) of dark-grown corn, black nightshade, morning glory, and velvetleaf seedlings as described previously (Grossmann et al. 2010). Before harvesting, the seedlings were allowed to green for 2 hours in the light in order to achieve the highest specific enzyme activities in the thylakoid fractions at low chlorophyll concentrations. At high chlorophyll concentrations significant quenching of fluorescence occurs, which limits the amount of green thylakoids that can be used in the test. Plant materials were homogenized in the cold with a Braun blender using a fresh-weight-to-volume ratio of 1:4. Homogenization buffer consisted of tris(hydroxymethyl)aminomethane (Tris)-HCl (50 mM; pH 7.3), sucrose (0.5 M), magnesium chloride (1 mM), ethylenediaminetetraacetic acid (EDTA) (1 mM) and bovine serum albumin (2 g L⁻¹). After filtration through four layers of Miracloth, crude plastid preparations were obtained after centrifugation at 10 000×g for 5 min and resuspension in homogenization buffer before centrifugation at 150×g for 2 min to remove crude cell debris. The supernatant was centrifuged at 4000×g for 15 min and the pellet fraction was resuspended in 1 ml of a buffer containing Tris-HCl (50 mM; pH 7.3), EDTA (2 mM), leupeptin (2 μM), pepstatin (2 μM) and glycerol (200 ml L⁻¹) and stored at −80° C. until use. Protein was determined in the enzyme extract with bovine serum albumin as a standard. PPO activity was assayed fluorometrically by monitoring the rate of Proto formation from chemically reduced protoporphyrinogen IX under initial velocity conditions. The assay mixture consisted of Tris-HCl (100 mM; pH 7.3), EDTA (1 mM), dithiothreitol (5 mM), Tween 80 (0.085%), protoporphyrinogen IX (2 μM), and 40 μg extracted protein in a total volume of 200 μl. The reaction was initiated by addition of substrate protoporphyrinogen IX at 22° C. saflufenacil, benzoxazinone-derivative herbicide, flumioxazin, butafenacil, acifluorfen, lactofen, bifenox, diuron, sulfentrazone were prepared in dimethyl sulfoxide (DMSO) solution (0.1 mM concentration of DMSO in the assay) and added to the assay mixture in concentrations of 0.005 pM to 5 μM before incubation. Fluorescence was monitored directly from the assay mixture using a POLARstar Optima/Galaxy (BMG) with excitation at 405 nm and emission monitored at 630 nm. Non-enzymatic activity in the presence of heat-inactivated extract was negligible. Inhibition of enzyme activity induced by the herbicide was expressed as percentage inhibition relative to untreated controls. Molar concentrations of compound required for 50% enzyme inhibition (IC₅₀ values) were calculated by fitting the values to the dose-response equation using non-linear regression analysis.

PPO Enzyme Assay (recombinant). Proto was purchased from Sigma-Aldrich (Milwaukee, WI). Protogen was prepared according to Jacobs and Jacobs (N. J. Jacobs, J. M. Jacobs, Assay for enzymatic protoporphyrinogen oxidation, a late step in heme synthesis, Enzyme 28 (1982) 206-219). Assays were conducted in 100 mM sodium phosphate pH 7.4 with 0.1 mM EDTA, 0.1% Tween 20, 5 μM FAD, and 500 mM imidazole. Dose-response curves with the PPO inhibitors saflufenacil, benzoxazinone-derivative herbicide, flumioxazin, butafenacil, acifluorfen, lactofen, bifenox, diuron, sulfentrazone, and MC-15608 were obtained in the presence of 150 μM Protogen. The excitation and emission bandwidths were set at 1.5 and 30 nm, respectively. All assays were made in duplicates or triplicates and measured using a POLARstar Optima/Galaxy (BMG) with excitation at 405 nm and emission monitored at 630 nm. Molar concentrations of compound required for 50% enzyme inhibition (IC₅₀ values) were calculated by fitting the values to the dose-response equation using non-linear regression analysis.

### Example 2. Engineering PPO-Derivative Herbicide Tolerant Plants Having Wildtype or Mutated PPO Sequences

PPO-derivative herbicide tolerant soybean (Glycine max) or corn (Zea mays) plants are produced by a method as described by Olhoft et al. (US patent 2009/0049567). For transformation of soybean or Arabidopsis thaliana, Wildtype or Mutated PPO sequences are cloned with standard cloning techniques as described in Sambrook et al. (Molecular cloning (2001) Cold Spring Harbor Laboratory Press) in a binary vector containing resistance marker gene cassette (AHAS) and mutated PPO sequence (marked as GOI) in between ubiquitin promoter (PcUbi) and nopaline synthase terminator (NOS) sequence. For corn transformation, Wildtype or Mutated PPO sequences are cloned with standard cloning techniques as described in Sambrook et al. (Molecular cloning (2001) Cold Spring Harbor Laboratory Press) in a binary vector containing resistance marker gene cassette (AHAS) and mutated PPO sequence (marked as GOI) in between corn ubiquitin promoter (ZmUbi) and nopaline synthase terminator (NOS) sequence. Binary plasmids are introduced to Agrobacterium tumefaciens for plant transformation. Plasmid constructs are introduced into soybean's axillary meristem cells at the primary node of seedling explants via Agrobacterium-mediated transformation. After inoculation and co-cultivation with Agrobacteria, the explants are transferred to shoot introduction media without selection for one week. The explants were subsequently transferred to a shoot induction medium with 1-3 μM imazapyr (Arsenal) for 3 weeks to select for transformed cells. Explants with healthy callus/shoot pads at the primary node are then transferred to shoot elongation medium containing 1-3 μM imazapyr until a shoot elongated or the explant died. Transgenic plantlets are rooted, subjected to TaqMan analysis for the presence of the transgene, transferred to soil and grown to maturity in greenhouse. Transformation of corn plants are done by a method described by McElver and Singh (WO 2008/124495). Plant transformation vector constructs containing mutated PPO sequences are introduced into maize immature embryos via Agrobacterium-mediated transformation.

Transformed cells were selected in selection media supplemented with 0.5-1.5 μM imazethapyr for 3-4 weeks. Transgenic plantlets were regenerated on plant regeneration media and rooted afterwards. Transgenic plantlets are subjected to TaqMan analysis for the presence of the transgene before being transplanted to potting mixture and grown to maturity in greenhouse. Arabidopsis thaliana are transformed with wildtype or mutated PPO sequences by floral dip method as described by McElver and Singh (WO 2008/124495). Transgenic Arabidopsis plants were subjected to TaqMan analysis for analysis of the number of integration loci. Transformation of Oryza sativa (rice) are done by protoplast transformation as described by Peng et al. (U.S. Pat. No. 6,653,529) T0 or T1 transgenic plant of soybean, corn, and rice containing mutated PPO sequences are tested for improved tolerance to PPO-derived herbicides in greenhouse studies and mini-plot studies with the following PPO-inhibiting herbicides: saflufenacil, benzoxazinone-derivative herbicide, flumioxazin, butafenacil, acifluorfen, lactofen, bifenox, diuron, sulfentrazone.

Transgenic Arabidopsis thaliana plants were assayed for improved tolerance to saflufenacil, benzoxazine-derivative herbicide, flumioxazin, butafenacil, acifluorfen, lactofen, bifenox, diuron, sulfentrazon in 48-well plates. Therefore, T2 seeds are surface sterilized by stirring for 5 min in ethanol+water (70+30 by volume), rinsing one time with ethanol+water (70+30 by volume) and two times with sterile, deionized water. The seeds are resuspended in 0.1% agar dissolved in water (w/v) Four to five seeds per well are plated on solid nutrient medium consisting of half-strength murashige skoog nutrient solution, pH 5.8 (Murashige and Skoog (1962) Physiologia Plantarum 15:473-497). Compounds are dissolved in dimethylsulfoxid (DMSO) and added to the medium prior solidification (final DMSO concentration 0.1%). Multi well plates are incubated in a growth chamber at 22° C., 75% relative humidity and 110 μmol Phot*m⁻²*s⁻¹ with 14:10 h light:dark photoperiod. Growth inhibition is evaluated seven to ten days after seeding in comparison to wild type plants. Additionally, transgenic T1 Arabidopsis plants were tested for improved tolerance to PPO-inhibiting herbicides in greenhouse studies with the following PPO-inhibiting herbicides: saflufenacil, benzoxazinone.derivative herbicide, flumioxazin, butafenacil, acifluorfen, lactofen, bifenox, diuron, sulfentrazone.

### Example 3. Tissue Culture Conditions

An in vitro tissue culture mutagenesis assay has been developed to isolate and characterize plant tissue (e.g., maize, rice tissue) that is tolerant to protoporphyrinogen oxidase inhibiting herbicides, (saflufenacil, benzoxazinone-derivative herbicide, flumioxazin, butafenacil, acifluorfen, lactofen, bifenox, diuron, sulfentrazone). The assay utilizes the somaclonal variation that is found in in vitro tissue culture. Spontaneous mutations derived from somaclonal variation can be enhanced by chemical mutagenesis and subsequent selection in a stepwise manner, on increasing concentrations of herbicide.

The present invention provides tissue culture conditions for encouraging growth of friable, embryogenic maize or rice callus that is regenerable. Calli were initiated from 4 different maize or rice cultivars encompassing Zea mays and Japonica (Taipei 309, Nipponbare, Koshihikari) and Indica (Indica 1) varieties, respectively. Seeds were surface sterilized in 70% ethanol for approximately 1 min followed by 20% commercial Clorox bleach for 20 minutes. Seeds were rinsed with sterile water and plated on callus induction media. Various callus induction media were tested. The ingredient lists for the media tested are presented in Table 4.

TABLE 4

| Ingredient | Supplier | R001M | | R025M | | R026M | | R327M | | R008M | | MS711R | |
|---|---|---|---|---|---|---|---|---|---|---|---|---|---|
| B5 Vitamins | Sigma | | | | | | | | | 1.0 | X | | |
| MS salts | Sigma | | | | | 1.0 | X | 1.0 | X | 1.0 | X | 1.0 | X |
| MS Vitamins | Sigma | | | | | 1.0 | X | 1.0 | X | | | | |
| N6 salts | Phytotech | 4.0 | g/L | 4.0 | g/L | | | | | | | | |
| N6 vitamins | Phytotech | 1.0 | X | 1.0 | X | | | | | | | | |
| L-Proline | Sigma | 2.9 | g/L | 0.5 | g/L | | | | | | | 1.2 | g/L |
| Casamino Acids | BD | 0.3 | g/L | 0.3 | g/L | 2 | g/L | | | | | | |
| Casein Hydrolysate | Sigma | | | | | | | | | | | 1.0 | g/L |
| L-Asp Monohydrate | Phytotech | | | | | | | | | | | 150 | mg/L |
| Nicotinic Acid | Sigma | | | | | | | | | | | 0.5 | mg/L |
| Pyridoxine HCl | Sigma | | | | | | | | | | | 0.5 | mg/L |
| Thiamine HCl | Sigma | | | | | | | | | | | 1.0 | mg/L |
| Myo-inositol | Sigma | | | | | | | | | | | 100 | mg/L |
| MES | Sigma | 500 | mg/L | 500 | mg/L | 500 | mg/L | 500 | mg/L | 500 | mg/L | 500 | mg/L |
| Maltose | VWR | 30 | g/L | 30 | g/L | 30 | g/L | 30 | g/L | | | | |
| Sorbitol | Duchefa | | | | | 30 | g/L | | | | | | |
| Sucrose | VWR | | | | | | | | | 10 | g/L | 30 | g/L |
| NAA | Duchefa | | | | | | | | | 50 | μg/L | | |
| 2,4-D | Sigma | 2.0 | mg/L | | | | | | | | | 1.0 | mg/L |
| MgCl₂•6H₂O | VWR | | | | | | | | | 750 | mg/L | | |
| →pH | | 5.8 | | 5.8 | | 5.8 | | 5.8 | | 5.8 | | 5.7 | |
| Gelrite | Duchefa | 4.0 | g/L | | | | | | | 2.5 | g/L | | |
| Agarose Type1 | Sigma | | | 7.0 | g/L | 10 | g/L | 10 | g/L | | | | |
| →Autoclave | | 15 | min | 15 | min | 15 | min | 15 | min | 15 | min | 20 | min |
| Kinetin | Sigma | | | 2.0 | mg/L | 2.0 | mg/L | | | | | | |
| NAA | Duchefa | | | 1.0 | mg/L | 1.0 | mg/L | | | | | | |
| ABA | Sigma | | | 5.0 | mg/L | | | | | | | | |
| Cefotaxime | Duchefa | | | 0.1 | g/L | 0.1 | g/L | 0.1 | g/L | | | | |
| Vancomycin | Duchefa | | | 0.1 | g/L | 0.1 | g/L | 0.1 | g/L | | | | |
| G418 Disulfate | Sigma | | | 20 | mg/L | 20 | mg/L | 20 | mg/L | | | | |

R001M callus induction media was selected after testing numerous variations. Cultures were kept in the dark at 30° C. Embryogenic callus was subcultured to fresh media after 10-14 days.

### Example 4. Selection of Herbicide-Tolerant Calli

Once tissue culture conditions were determined, further establishment of selection conditions were established through the analysis of tissue survival in kill curves with saflufenacil, benzoxazinone-derivative herbicide, flumioxazin, butafenacil, acifluorfen, lactofen, bifenox, diuron, sulfentrazone. Careful consideration of accumulation of the herbicide in the tissue, as well as its persistence and stability in the cells and the culture media was performed. Through these experiments, a sub-lethal dose has been established for the initial selection of mutated material.

After the establishment of the starting dose of saflufenacil, benzoxazinone-derivative herbicide, flumioxazin, butafenacil, acifluorfen, lactofen, bifenox, diuron, sulfentrazone in selection media, the tissues were selected in a step-wise fashion by increasing the concentration of the PPO inhibitor with each transfer until cells are recovered that grew vigorously in the presence of toxic doses. The resulting calli were further subcultured every 3-4 weeks to R001M with selective agent. Over 26,000 calli were subjected to selection for 4-5 subcultures until the selective pressure was above toxic levels as determined by kill curves and observations of continued culture.

Alternatively, liquid cultures initiated from calli in MS711R with slow shaking and weekly subcultures. Once liquid cultures were established, selection agent was added directly to the flask at each subculture. Following 2-4 rounds of liquid selection, cultures were transferred to filters on solid R001M media for further growth.

### Example 5. Regeneration of Plants

Tolerant tissue was regenerated and characterized molecularly for PPO gene sequence mutations and/or biochemically for altered PPO activity in the presence of the selective agent. In addition, genes involved directly and/or indirectly in tetrapyrrole biosynthesis and/or metabolism pathways were also sequenced to characterize mutations. Finally, enzymes that change the fate (e.g. metabolism, translocation, transportaion) were also sequence to characterized mutations.

Following herbicide selection, calli were regenerated using a media regime of R025M for 10-14 days, R026M for ca. 2 weeks, R327M until well formed shoots were developed, and R008S until shoots were well rooted for transfer to the greenhouse. Regeneration was carried out in the light. No selection agent was included during regeneration.

Once strong roots were established, MO regenerants were transplant to the greenhouse in square or round pots. Transplants were maintained under a clear plastic cup until they were adapted to greenhouse conditions. The greenhouse was set to a day/night cycle of 27° C./21° C. (80° F./70° F.) with 600 W high pressure sodium lights supplementing light to maintain a 14 hour day length. Plants were watered according to need, depending in the weather and fertilized daily.

### Example 6. Sequence Analysis

Leaf tissue was collected from clonal plants separated for transplanting and analyzed as individuals. Genomic DNA was extracted using a Wizard® 96 Magnetic DNA Plant System kit (Promega, U.S. Pat. Nos. 6,027,945 & 6,368,800) as directed by the manufacturer. Isolated DNA was PCR amplified using the appropriate forward and reverse primer.

PCR amplification was performed using Hotstar Taq DNA Polymerase (Qiagen) using touchdown thermocycling program as follows: 96° C. for 15 min, followed by 35 cycles (96° C., 30 sec; 58° C.-0.2° C. per cycle, 30 sec; 72° C., 3 min and 30 sec), 10 min at 72° C.

PCR products were verified for concentration and fragment size via agarose gel electrophoresis. Dephosphorylated PCR products were analyzed by direct sequence using the PCR primers (DNA Landmarks, or Entelechon). Chromatogram trace files (.scf) were analyzed for mutation relative to the wild-type gene using Vector NTI Advance 10™ (Invitrogen). Based on sequence information, mutations were identified in several individuals. Sequence analysis was performed on the representative chromatograms and corresponding AlignX alignment with default settings and edited to call secondary peaks.

### Example 7. Demonstration of Herbicide-Tolerance

Selected mutants and escapes were transferred to small pots. Wild-type cultivars were germinated from seed to serve as controls.

After ca. 3 weeks post-transplant, MO regenerants were sprayed using a track sprayer with saflufenacil, benzoxazinone-derivative herbicide, flumioxazin, butafenacil, acifluorfen, lactofen, bifenox, diuron, sulfentrazone supplemented with 0.1% methylated seed oil. After the plants had adapted to greenhouse conditions, a subset were sprayed with additional saflufenacil or benzoxazinone-derivative herbicide. Once sprayed, plants were kept on drought conditions for 24 hours before being watered and fertilized again. Sprayed plants were photographed and rated for herbicide injury at 1 and 3 weeks after treatment. No injury was observed on plants containing the heterozygous mutation while control plants and tissue culture escapes (regenerated plants negative for the sequenced mutations) were heavily damaged after treatment.

### Example 8. Herbicide Selection Using Tissue Culture

Media was selected for use and kill curves developed as specified above. For selection, different techniques were utilized. Either a step wise selection was applied, or an immediate lethal level of herbicide was applied. In either case, all of the calli were transferred for each new round of selection. Selection was 4-5 cycles of culture with 3-5 weeks for each cycle. Cali were placed onto nylon membranes to facilitate transfer (200 micron pore sheets, Biodesign, Saco, Maine). Membranes were cut to fit 100×20 mm Petri dishes and were autoclaved prior to use 25-35 calli (average weight/calli being 22 mg) were utilized in every plate. In addition, one set of calli were subjected to selection in liquid culture media with weekly subcultures followed by further selection on semi-solid media.

Mutant lines were selected using saflufenacil, benzoxazinone-derivative herbicide, flumioxazin, butafenacil, acifluorfen, lactofen, bifenox, diuron, sulfentrazone. Efficiencies of obtaining mutants was high either based on a percentage of calli that gave rise to a regenerable, mutant line or the number of lines as determined by the gram of tissue utilized.

### Example 9 Screening of Mutagenized Algae Cells to Identify Herbicide Tolerant Clones and Identification of Causative Mutations in PPO Genes

To generate mutations in PPO genes conferring “PPO-inhibiting herbicides” resistance, screening with chemical or UV mutagenized cell populations can be used. Especially unicellular organisms like Chlamydomonas reinhardtii are useful for identifying dominant mutations conferring herbicide resistance (Kataoka M, et al.; 1990; J. of Pest. Sci. 15:449-451; Oshio H, et al.; 1993; Zeitschrift für Naturforschung 48:339-344).

Algae cells of Chlamydomonas reinhardtii strains CC-503 and CC-1691 (Duke University, Durham, USA) were propagated in TAP medium (Gorman and Levine; 1965; PNAS 54:1665-1669) by constant shaking at 100 rpm, 22° C. and 30 μmol Phot*m⁻²*s⁻² light illumination. Compound screening was performed at 450 μmol Phot*m⁻²*s⁻² illumination.

Sensitive strains of Chlamydomonas reinhardtii were mutagenized with 0.14 M ethylmethanesulfonate (EMS) for 1 h as described by Loppes (1969, Mol Gen Genet 104:172-177). Tolerant strains are identified by screening of mutagenized cells on solid TAP medium plates containing “PPO-inhibiting herbicide” like saflufenacil or benzoxazinone-derivative herbicide at wildype-lethal concentrations depending on compound activity in CC-503 or CC-1691 strain.

Standard techniques were used for isolation of RNA and cDNA synthesis as described by Sambrock et al. (Molecular cloning (2001) Cold Spring Harbor Laboratory Press). Amplification of PPO genes from wild-type and resistant Chlamydomonas reinhardii from genomic DNA or copy DNA as template were performed by standard PCR techniques with DNA oligonucleotides as listed in Table 5. The resulting DNA molecules were cloned in standard sequencing vector (pJET1) and sequenced by standard sequencing techniques. Mutations were identified by comparing wildtype and mutant PPO sequences by sequence alignment tool Align X (Vector NTI Advance Software Version 10.3, Invitrogen, Carlsbad, CA, USA).

TABLE 5
PCR primer for amplification of CrPPO

| Primer name | Primer sequence (5′-3′) |
|---|---|
| Cr_PPO1_Fw | ATGATGTTGACCCAGACTCCTGGGAC |
| Cr_PPO1_Rv | TTAGGCCTTGACTGCGGCCTTGGAC |

1. A method for controlling undesired vegetation at a plant cultivation site, the method comprising the steps of:
a) providing, at said site, a plant that comprises at least one nucleic acid comprising
a nucleotide sequence encoding a wild-type protoporphyrinogen oxidase or a mutated protoporphyrinogen oxidase (mut-PPO) which is resistant or tolerant to a benzoxazinone-derivative herbicide and/or
b) applying to said site an effective amount of said herbicide.

2. The method according to claim 1, wherein the nucleotide sequence of a) comprises the sequence of SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof.

3. The method of claim 1, wherein the plant comprises at least one additional heterologous nucleic acid comprising a nucleotide sequence encoding a herbicide tolerance enzyme.

4. The method of claim 1, wherein the benzoxazinone-derivative herbicide is applied in conjunction with one or more other PPO targeting herbicides.

5. A method for identifying a benzoxazinone-derivative herbicide by using a mut-PPO encoded by a nucleic acid which comprises the nucleotide sequence of SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof.

6. The method of claim 5, comprising the steps of:
a) generating a transgenic cell or plant comprising a nucleic acid encoding a mut-PPO, wherein the mut-PPO is expressed;
b) applying a benzoxazinone-derivative to the transgenic cell or plant of a) and to a control cell or plant of the same variety;
c) determining the growth or the viability of the transgenic cell or plant and the control cell or plant after application of said test compound, and
d) selecting test compounds which confer reduced growth to the control cell or plant as compared to the growth of the transgenic cell or plant.

7. A method of identifying a nucleotide sequence encoding a mut-PPO which is resistant or tolerant to a benzoxazinone-derivative herbicide, the method comprising:
a) generating a library of mut-PPO-encoding nucleic acids,
b) screening a population of the resulting mut-PPO-encoding nucleic acids by expressing each of said nucleic acids in a cell or plant and treating said cell or plant with a benzoxazinone-derivative
c) comparing the benzoxazinone-derivative tolerance levels provided by said population of mut-PPO encoding nucleic acids with the benzoxazinone-derivative tolerance level provided by a control PPO-encoding nucleic acid,
d) selecting at least one mut-PPO-encoding nucleic acid that provides a significantly increased level of tolerance to a benzoxazinone-derivative as compared to that provided by the control PPO-encoding nucleic acid.

8. The method of claim 7, wherein the mut-PPO-encoding nucleic acid selected in step d) provides at least 2-fold as much tolerance to a benzoxazinone-derivative herbicide as compared to that provided by the control PPO-encoding nucleic acid.

9. The method of claim 7, wherein the resistance or tolerance is determined by generating a transgenic plant comprising a nucleic acid sequence of the library of step a) and comparing said transgenic plant with a control plant.

10. An isolated nucleic acid encoding a mut-PPO polypeptide, wherein the nucleic acid comprises the nucleotide sequence of SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof, and wherein the nucleic acid is identifiable by the method of claim 7.

11. The nucleic acid of claim 10, wherein the encoded mut-PPO is a variant of SEQ ID NO: 2 or SEQ ID NO. 4, which includes one or more of the following: the amino acid at position 128 is other than Arginine; the amino acid at position 204 is other than Phenylalanine, the amino acid at position 208 is other than Threonine; the amino acid at position 397 is other than Leucine, the amino acid at position 400 is other than Leucine, the amino acid at position 420 is other than Phenylalanine, the amino acid at position 457 is other than Phenylalanine.

12. A mut-PPO polypeptide comprising a sequence of SEQ ID NO: 2 or SEQ ID NO:4, a variant, derivative, othologue, paralogue or homologue thereof, in which the amino acid at position 420 is Met or Val, and the the amino acid at position 397 is Gly, Ala, Ser, Thr, Cys, Val, Ile, Met, Pro, Phe, Tyr, Trp, His, Lys, Arg, Asn, Asp, Glu, or Gin, wherein said mut-PPO polypeptide confers increased resistance or tolerance to a benzoxazinone-derivative herbicide in a plant as compared to a wild type plant.

13. A transgenic plant cell transformed by a nucleic acid encoding a mut-PPO polypeptide, wherein expression of the nucleic acid in the plant cell results in increased resistance or tolerance to a benzoxazinone-derivative herbicide as compared to a wild type variety of the plant cell.

14. The transgenic plant cell of claim 13, wherein the mut-PPO polypeptide encoding nucleic acid comprises a polynucleotide sequence selected from the group consisting of: a) a polynucleotide as shown in SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof; b) a polynucleotide encoding a polypeptide as shown in SEQ ID NO: 2, 4, 6, 8, 10, 12, 14, 16, 18, 20, 22, 24, 26, 28, 30, 32, 34, 36, 38, 40, 42, 44, or 46, or a variant or derivative thereof; c) a polynucleotide comprising at least 60 consecutive nucleotides of any of a) or b); and d) a polynucleotide complementary to the polynucleotide of any of a) through c).

15. The transgenic plant cell of claim 14, wherein the encoded mut-PPO is a variant of SEQ ID NO: 2 or SEQ ID NO. 4, which includes one or more of the following: the amino acid at position 128 is other than Arginine; the amino acid at position 204 is other than Phenylalanine, the amino acid at position 208 is other than Threonine; the amino acid at position 397 is other than Leucine, the amino acid at position 400 is other than Leucine, the amino acid at position 420 is other than Phenylalanine, the amino acid at position 457 is other than Phenylalanine.

16. A transgenic plant comprising the plant cell of claim 13, wherein expression of the mut-PPO polypeptide encoding nucleic acid in the plant results in the plant's increased resistance to benzoxazinone-derivative herbicide as compared to a wild type plant.

17. A plant that expresses a mutagenized or recombinant mut-PPO polypeptide comprising the sequence of SEQ ID NO: 2 or SEQ ID NO:4, a variant, derivative, othologue, paralogue or homologue thereof, in which the amino acid at position 420 is Met or Val, and the the amino acid at position 397 is Gly, Ala, Ser, Thr, Cys, Val, Ile, Met, Pro, Phe, Tyr, Trp, His, Lys, Arg, Asn, Asp, Glu, or Gin, and wherein said mut-PPO confers upon the plant increased herbicide tolerance as compared to the corresponding wild-type variety of the plant when expressed therein.

18. A seed produced by a transgenic plant comprising the plant cell of claim 13, wherein the seed is true breeding for an increased resistance to a benzoxazinone-derivative herbicide as compared to a wild type variety of the seed.

19. A method of producing a transgenic plant cell with an increased resistance to a benzoxazinone-derivative herbicide as compared to a wild type variety of the plant cell comprising, transforming the plant cell with an expression cassette comprising a nucleic acid encoding a mut-PPO polypeptide.

20. A method of producing a transgenic plant comprising, (a) transforming a plant cell with an expression cassette comprising a nucleic acid encoding a mut-PPO polypeptide, and (b) generating a plant with an increased resistance to benzoxazinone-derivative herbicide from the plant cell.

21. The method of claim 19, wherein the mut-PPO nucleic acid comprises a polynucleotide sequence selected from the group consisting of: a) a polynucleotide as shown in SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof; b) a polynucleotide encoding a polypeptide as shown in SEQ ID NO: 2, 4, 6, 8, 10, 12, 14, 16, 18, 20, 22, 24, 26, 28, 30, 32, 34, 36, 38, 40, 42, 44, or 46, or a variant or derivative thereof; c) a polynucleotide comprising at least 60 consecutive nucleotides of any of a) or b); and d) a polynucleotide complementary to the polynucleotide of any of a) through c).

22. The method of claim 19, wherein the expression cassette further comprises a transcription initiation regulatory region and a translation initiation regulatory region that are functional in the plant.

23. A method of identifying or selecting a transformed plant cell, plant tissue, plant or part thereof comprising: i) providing a transformed plant cell, plant tissue, plant or part thereof, wherein said transformed plant cell, plant tissue, plant or part thereof comprises a polynucleotide as shown in SEQ ID NO: 1, 3, 5, 7, 9, 11, 13, 15, 17, 19, 21, 23, 25, 27, 29, 31, 33, 35, 37, 39, 41, 43, or 45, or a variant or derivative thereof, wherein the polynucleotide encodes an mut-PPO polypeptide that is used as a selection marker, and wherein said transformed plant cell, plant tissue, plant or part thereof may comprise a further isolated polynucleotide; ii) contacting the transformed plant cell, plant tissue, plant or part thereof with at least one benzoxazinine-derivative compound; iii) determining whether the plant cell, plant tissue, plant or part thereof is affected by the inhibiting compound; and iv) identifying or selecting the transformed plant cell, plant tissue, plant or part thereof.


SEQUENCE LISTING (48 sequences)

SEQ ID NO: 1 - DNA, 1605 bp, Amaranthus tuberculatus
  source 1..1605: mol_type = other DNA
atggtaattcaatccattacccacctttcaccaaaccttgcattgccatcgccattgtcagtttcaaccaagaactacccagtagctgtaatgggcaacatttctgagcgggaagaacccacttctgctaaaagggttgctgttgttggtgctggagttagtggacttgctgctgcatataagctaaaatcccatggtttgagtgtgacattgtttgaagctgattctagagctggaggcaaacttaaaactgttaaaaaagatggttttatttgggatgagggggcaaatactatgacagaaagtgaggcagaggtctcgagtttgatcgatgatcttgggcttcgtgagaagcaacagttgccaatttcacaaaataaaagatacatagctagagacggtcttcctgtgctactaccttcaaatcccgctgcactactcacgagcaatatcctttcagcaaaatcaaagctgcaaattatgttggaaccatttctctggagaaaacacaatgctactgaactttctgatgagcatgttcaggaaagcgttggtgaattttttgagcgacattttgggaaagagtttgttgattatgttatcgacccttttgttgcgggtacatgtggtggagatcctcaatcgctttccatgcaccatacatttccagaagtatggaatattgaaaaaaggtttggctctgtgtttgctggactaattcaatcaacattgttatctaagaaggaaaagggtggagaaaatgcttctattaagaagcctcgtgtacgtggttcattttcatttcaaggtggaatgcagacacttgttgacacaatgtgcaaacagcttggtgaagatgaactcaaactccagtgtgaggtgctgtccttgtcatataaccagaaggggatcccctcattagggaattggtcagtctcttctatgtcaaataataccagtgaagatcaatcttatgatgctgtggttgtcactgctccaattcgcaatgtcaaagaaatgaagattatgaaatttggaaatccattttcacttgactttattccagaggtgacgtacgtacccctttccgttatgattactgcattcaaaaaggataaagtgaagagacctcttgagggcttcggagttcttatcccctctaaagagcaacataatggactgaagactcttggtactttattttcctccatgatgtttcctgatcgtgctccatctgacatgtgtctctttactacatttgtcggaggaagcagaaatagaaaacttgcaaacgcttcaacggatgaattgaagcaaatagtttcttctgaccttcagcagctgttgggcactgaggacgaaccttcatttgtcaatcatctcttttggagcaacgcattcccattgtatggacacaattacgattctgttttgagagccatagacaagatggaaaaggatcttcctggatttttttatgcaggtaaccataagggtggactttcagtgggaaaagcgatggcctccggatgcaaggctgcggaacttgtaatatcctatctggactctcatatatacgtgaagatggatgagaagaccgcgtaa

SEQ ID NO: 2 - protein, 534 aa, Amaranthus tuberculatus
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 3 - DNA, 1605 bp, Amaranthus tuberculatus
  source 1..1605: mol_type = other DNA
atggtaattcaatccattacccacctttcaccaaaccttgcattgccatcgccattgtcagtttcaaccaagaactacccagtagctgtaatgggcaacatttctgagcgggaagaacccacttctgctaaaagggttgctgttgttggtgctggagttagtggacttgctgctgcatataagctaaaatcccatggtttgagtgtgacattgtttgaagctgattctagagctggaggcaaacttaaaactgttaaaaaagatggttttatttgggatgagggggcaaatactatgacagaaagtgaggcagaggtctcgagtttgatcgatgatcttgggcttcgtgagaagcaacagttgccaatttcacaaaataaaagatacatagctagagccggtcttcctgtgctactaccttcaaatcccgctgcactactcacgagcaatatcctttcagcaaaatcaaagctgcaaattatgttggaaccatttctctggagaaaacacaatgctactgaactttctgatgagcatgttcaggaaagcgttggtgaattttttgagcgacattttgggaaagagtttgttgattatgttattgacccttttgttgcgggtacatgtggtggagatcctcaatcgctttccatgcaccatacatttccagaagtatggaatattgaaaaaaggtttggctctgtgtttgccggactaattcaatcaacattgttatctaagaaggaaaagggtggagaaaatgcttctattaagaagcctcgtgtacgtggttcattttcatttcaaggtggaatgcagacacttgttgacacaatgtgcaaacagcttggtgaagatgaactcaaactccagtgtgaggtgctgtccttgtcatataaccagaaggggatcccctcactagggaattggtcagtctcttctatgtcaaataataccagtgaagatcaatcttatgatgctgtggttgtcactgctccaattcgcaatgtcaaagaaatgaagattatgaaatttggaaatccattttcacttgactttattccagaggtgacgtacgtacccctttccgttatgattactgcattcaaaaaggataaagtgaagagacctcttgagggcttcggagttcttatcccctctaaagagcaacataatggactgaagactcttggtactttattttcctccatgatgtttcctgatcgtgctccatctgacatgtgtctctttactacatttgtcggaggaagcagaaatagaaaacttgcaaacgcttcaacggatgaattgaagcaaatagtttcttctgaccttcagcagctgttgggcactgaggacgaaccttcatttgtcaatcatctcttttggagcaacgcattcccattgtatggacacaattacgattctgttttgagagccatagacaagatggaaaaggatcttcctggatttttttatgcaggtaaccataagggtggactttcagtgggaaaagcgatggcctccggatgcaaggctgcggaacttgtaatatcctatctggactctcatatatacgtgaagatggatgagaagaccgcgtaa

SEQ ID NO: 4 - protein, 534 aa, Amaranthus tuberculatus
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARAGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 5 - DNA, 1602 bp, Amaranthus tuberculatus
  source 1..1602: mol_type = other DNA
atggtaattcaatccattacccacctttcaccaaaccttgcattgccatcgccattgtcagtttccaccaagaactacccagtagctgtaatgggcaacatttctgagcgagaagaacccacttctgctaaaagggttgctgttgttggtgctggagttagtggacttgctgctgcatataagctaaaatcccatggtttgagtgtgacattgtttgaagctgattctagagctggaggcaaacttaaaactgttaaaaaagatggttttatttgggatgagggggcaaatactatgacagaaagtgaggcagaggtctcgagtttgatcgatgatcttgggcttcgtgagaagcaacagttgccaatttcacaaaataaaagatacatagctagagacggtcttcctgtgctactaccttcaaatcccgctgcactactcacgagcaatatcctttcagcaaaatcaaagctgcaaattatgttggaaccatttctctggagaaaacacaatgctactgaactttctgatgagcatgttcaggaaagcgttggtgaattttttgagcgacattttgggaaagagtttgttgattatgttattgacccttttgttgcgggtacatgtggagatcctcaatcgctttccatgcaccatacatttccagaagtatggaatattgaaaaaaggtttggctctgtgtttgctggactaattcaatcaacattgttatctaagaaggaaaagggtggagaaaatgcttctattaagaagcctcgtgtacgtggttcattttcatttcaaggtggaatgcagacacttgttgacacaatgtgcaaacagcttggtgaagatgaactcaaactccagtgtgaggtgctgtccttgtcatataaccagaaggggatcccctcattagggaattggtcagtctcttctatgtcaaataataccagtgaagatcaatcttatgatgctgtggttgtcactgctccaattcgcaatgtcaaagaaatgaagattatgaaatttggaaatccattttcacttgactttattccagaggtgacgtacgtacccctttccgttatgattactgcattcaaaaaggataaagtgaagagacctcttgagggcttcggagttcttatcccctctaaagagcaacataatggactgaagactcttggtactttattttcctccatgatgtttcctgatcgtgctccatctgacatgtgtctctttactacatttgtcggaggaagcagaaatagaaaacttgcaaacgcttcaacggatgaattgaagcaaatagtttcttctgaccttcagcagctgttgggcactgaggacgaaccttcatttgtcaatcatctcttttggagcaacgcattcccattgtatggacacaattacgattgtgttttgagagccatagacaagatggaaaaggatcttcctggatttttttatgcaggtaaccataagggtggactttcagtgggaaaagcgatggcctccggatgcaaggctgcggaacttgtaatatcctatctggactctcatatatacgtgaagatggatgagaagaccgcgtaa

SEQ ID NO: 6 - protein, 533 aa, Amaranthus tuberculatus
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDCVLRAIDKMEKDLPGFFYAGNHKGGLSVGKAMASGCKAAELVISYLDSHIYVKMDEKTA

SEQ ID NO: 7 - DNA, 1602 bp, Amaranthus tuberculatus
  source 1..1602: mol_type = other DNA
atggtaattcaatccattacccacctttcaccaaaccttgcattgccatcgccattgtcagtttccaccaagaactacccagtagctgtaatgggcaacatttctgagcgggaagaacccacttctgctaaaagggttgctgttgttggtgctggagttagtggacttgctgctgcatataagctaaaatcccatggtttgagtgtgacattgtttgaagctaattctagagctggaggcaaacttaaaactgttaaaaaagatggttttatttgggatgagggggcaaatactatgacagaaagtgaggcagaggtctcgagtttgatcgatgatcttgggcttcgtgagaagcaacagttgccaatttcacaaaataaaagatacatagctagagacggtcttcctgtgctactaccttcaaatcccgctgcactactcacgagcaatatcctttcagcaaaatcaaagctgcaaattatgttggaaccatttctctggagaaaacacaatgctactgaactttctgatgagcatgttcaggaaagcgttggtgaattttttgagcgacattttgggaaagagtttgttgattatgttattgacccttttgttgcgggtacatgtggagatcctcaatcgctttccatgtaccatacatttccagaagtatggaatattgaaaaaaggtttggctctgtgtttgctggactaattcaatcaacattgttatctaagaaggaaaagggtggagaaaatgcttctattaagaagcctcgtgtacgtggttcattttcatttcaaggtggaatgcagacacttgttgacacaatgtgcaaacagcttggtgaagatgaactcaaactccagtgtgaggtgctgtccttgtcatataaccagaaggggatcccctcattagggaattggtcagtctcttctatgtcaaataataccagtgaagatcaatcttatgatgctgtggttgtcactgctccaattcgcaatgtcaaagaaatgaagattatgaaatttggaaatccattttcacttgactttattccagaggtgacgtacgtacccctttccgttatgattactgcattcaaaaaggataaagtgaagagacctcttgagggcttcggagttcttatcccctctaaagagcaacataatggactgaagactcttggtactttattttcctccatgatgtttcctgatcgtgctccatctgacatgtgtctctttactacatttgtcggaggaagcagaaatagaaaacttgcaaacgcttcaacggatgaattgaagcaaatagtttcttctgaccttcagcagctgttgggcactgaggacgaaccttcatttgtcaatcatctcttttggagcaacgcattcccattgtatggacacaattacgattctgttttgagagccatagacaagatggaaaaggatcttcctggatttttttatgcaggtaaccataagggtggactttcagtgggaaaagcgatggcctccggatgcaaggctgcggaacttgtaatatcctatctggactctcatatatacgtgaagatggatgagaagaccgcgtaa

SEQ ID NO: 8 - protein, 533 aa, Amaranthus tuberculatus
MVIQSITHLSPNLALPSPLSVSTKNYPVAVMGNISEREEPTSAKRVAVVGAGVSGLAAAYKLKSHGLSVTLFEANSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGDPQSLSMYHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 9 - DNA, 1644 bp, Arabidopsis thaliana
  source 1..1644: mol_type = other DNA
atgggcctgattaaaaacggtaccctttattgtcgttttgggataagctggaattttgccgctgtgtttttttctacttatttccgtcactgctttcgactggtcagagattttgactctgaattgttgcagatagcaatggcgtctggagcagtagcagatcatcaaattgaagcggtttcaggaaaaagagtcgcagtcgtaggtgcaggtgtaagtggacttgcggcggcttacaagttgaaatcgaggggtttgaatgtgactgtgtttgaagctgatggaagagtaggtgggaagttgagaagtgttatgcaaaatggtttgatttgggatgaaggagcaaacaccatgactgaggctgagccagaagttgggagtttacttgatgatcttgggcttcgtgagaaacaacaatttccaatttcacagaaaaagcggtatattgtgcggaatggtgtacctgtgatgctacctaccaatcccatagagctggtcacaagtagtgtgctctctacccaatctaagtttcaaatcttgttggaaccatttttatggaagaaaaagtcctcaaaagtctcagatgcatctgctgaagaaagtgtaagcgagttctttcaacgccattttggacaagaggttgttgactatctcatcgacccttttgttggtggaacaagtgctgcggaccctgattccctttcaatgaagcattctttcccagatctctggaatagttttggctctattatagtcggtgcaatcagaacaaagtttgctgctaaaggtggtaaaagtagagacacaaagagttctcctggcacaaaaaagggttcgcgtgggtcattctcttttaaggggggaatgcagattcttcctgatacgttgtgcaaaagtctctcacatgatgagatcaatttagactccaaggtactctctttgtcttacaattctggatcaagacaggagaactggtcattatcttgtgtttcgcataatgaaacgcagagacaaaacccccattatgatgctgctcctctgtgcaatgtgaaggagatgaaggttatgaaaggaggacaaccctttcagctaaactttctccccgagattaattacatgcccctctcggttttaatcaccacattcacaaaggagaaagtaaagagacctcttgaaggctttggggtactcattccatctaaggagcaaaagcatggtttcaaaactctaggtacacttttttcatcaatgatgtttccagatcgttcccctagtgacgttcatctatatacaacttttattggtgggagtaggaaccaggaactagccaaagcttccactgacgaattaaaacaagttgtgacttctgaccttcagcgactgttgggggttgaaggtgaacccgtgtctgtcaaccattactattggaggaaagcattcccgttgtatgacagcagctatgactcagtcatggaagcaattgacaagatggagaatgatctacctgggttcttctatgcaggtaatcatcgaggggggctctctgttgggaaatcaatagcatcaggttgcaaagcagctgaccttgtgatctcatacctggagtcttgctcaaatgacaagaaaccaaatgacagcttataa

SEQ ID NO: 10 - protein, 547 aa, Arabidopsis thaliana
MGLIKNGTLYCRFGISWNFAAVFFSTYFRHCFRLVRDFDSELLQIAMASGAVADHQIEAVSGKRVAVVGAGVSGLAAAYKLKSRGLNVTVFEADGRVGGKLRSVMQNGLIWDEGANTMTEAEPEVGSLLDDLGLREKQQFPISQKKRYIVRNGVPVMLPTNPIELVTSSVLSTQSKFQILLEPFLWKKKSSKVSDASAEESVSEFFQRHFGQEVVDYLIDPFVGGTSAADPDSLSMKHSFPDLWNSFGSIIVGAIRTKFAAKGGKSRDTKSSPGTKKGSRGSFSFKGGMQILPDTLCKSLSHDEINLDSKVLSLSYNSGSRQENWSLSCVSHNETQRQNPHYDAAPLCNVKEMKVMKGGQPFQLNFLPEINYMPLSVLITTFTKEKVKRPLEGFGVLIPSKEQKHGFKTLGTLFSSMMFPDRSPSDVHLYTTFIGGSRNQELAKASTDELKQVVTSDLQRLLGVEGEPVSVNHYYWRKAFPLYDSSYDSVMEAIDKMENDLPGFFYAGNHRGGLSVGKSIASGCKAADLVISYLESCSNDKKPNDSL

SEQ ID NO: 11 - DNA, 1647 bp, Nicotiana tabacum
  source 1..1647: mol_type = other DNA
atgacaacaactcccatcgccaatcatcctaatattttcactcaccagtcgtcgtcatcgccattggcattcttaaaccgtacgagtttcatccctttctcttcaatctccaagcgcaatagtgtcaattgcaatggctggagaacacgatgctccgttgccaaagattacacagttccttcctcagcggtcgacggcggacccgccgcggagctggactgtgttatagttggagcaggaattagtggcctctgcattgcgcaggtgatgtccgctaattaccccaatttgatggtaaccgaggcgagagatcgtgccggtggcaacataacgactgtggaaagagacggctatttgtgggaagaaggtcccaacagtttccagccgtccgatcctatgttgactatggcagtagattgtggattgaaggatgatttggtgttgggagatcctaatgcgccccgtttcgttttgtggaagggtaaattaaggcccgtcccctcaaaactcactgatcttcccttttttgatttgatgagcattcctggcaagttgagagctggttttggtgccattggcctccgcccttcacctccaggtcatgaggaatcagttgagcagttcgtgcgtcgtaatcttggtggcgaagtctttgaacgcttgatagaaccattttgttctggtgtttatgctggtgatccctcaaaactgagtatgaaagcagcatttgggaaagtttggaagttggaagaaactggtggtagcattattggaggaacctttaaagcaataaaggagagatccagtacacctaaagcgccccgcgatccgcgtttacctaaaccaaaaggacagacagttggatcattcaggaagggtctcagaatgctgccggatgcaatcagtgcaagattgggaagcaaattaaaactatcatggaagctttctagcattactaagtcagaaaaaggaggatatcacttgacatacgagacaccagaaggagtagtttctcttcaaagtcgaagcattgtcatgactgtgccatcctatgtagcaagcaacatattacgtcctctttcggttgccgcagcagatgcactttcaaatttctactatcccccagttggagcagtcacaatttcatatcctcaagaagctattcgtgatgagcgtctggttgatggtgaactaaagggatttgggcagttgcatccacgtacacagggagtggaaacactaggaacgatatatagttcatcactcttccctaaccgtgccccaaaaggtcgggtgctactcttgaactacattggaggagcaaaaaatcctgaaattttgtctaagacggagagccaacttgtggaagtagttgatcgtgacctcagaaaaatgcttataaaacccaaagctcaagatcctcttgttgtgggtgtgcgagtatggccacaagctatcccacagtttttggttggtcatctggatacgctaagtactgcaaaagctgctatgaatgataatgggcttgaagggctgtttcttgggggtaattatgtgtcaggtgtagcattggggaggtgtgttgaaggtgcttatgaagttgcatccgaggtaacaggatttctgtctcggtatgcatacaaatga

SEQ ID NO: 12 - protein, 548 aa, Nicotiana tabacum
MTTTPIANHPNIFTHQSSSSPLAFLNRTSFIPFSSISKRNSVNCNGWRTRCSVAKDYTVPSSAVDGGPAAELDCVIVGAGISGLCIAQVMSANYPNLMVTEARDRAGGNITTVERDGYLWEEGPNSFQPSDPMLTMAVDCGLKDDLVLGDPNAPRFVLWKGKLRPVPSKLTDLPFFDLMSIPGKLRAGFGAIGLRPSPPGHEESVEQFVRRNLGGEVFERLIEPFCSGVYAGDPSKLSMKAAFGKVWKLEETGGSIIGGTFKAIKERSSTPKAPRDPRLPKPKGQTVGSFRKGLRMLPDAISARLGSKLKLSWKLSSITKSEKGGYHLTYETPEGVVSLQSRSIVMTVPSYVASNILRPLSVAAADALSNFYYPPVGAVTISYPQEAIRDERLVDGELKGFGQLHPRTQGVETLGTIYSSSLFPNRAPKGRVLLLNYIGGAKNPEILSKTESQLVEVVDRDLRKMLIKPKAQDPLVVGVRVWPQAIPQFLVGHLDTLSTAKAAMNDNGLEGLFLGGNYVSGVALGRCVEGAYEVASEVTGFLSRYAYK

SEQ ID NO: 13 - DNA, 1668 bp, Cichorium intybus
  source 1..1668: mol_type = other DNA
atgacatctctcacagacgtttgttccctcaactgttgccgtagctggtcttcccttccgccaccggtttctggtgggtcgttgacgtcaaagaatcctaggtacctaatcacgtatagtccggcgcatcgcaaatgcaataggtggaggttccgctgctctatagccaaggattccccaattactcctcccatttcaaatgagttcaactctcagccattgttggactgtgtcattgtgggcgccggcattagcggcctttgcattgcgcaggccctagcgactaaacacgcctccgtctctccggatgtgatcgtcaccgaggcacgagacagagtcgggggtaatatatcaacggttgaaagggatggctatctctgggaagaaggtcctaacagcttccagccatctgatgccatgctcaccatggtggtggatagtgggttgaaggatgatttggtgttaggtgacccaacagcaccccgctttgtattatggggaggtgatttgaaaccggttccttccaaaccggctgacctccctttctttgacctcatgagctttcctggaaaactcagagccggttttggtgctcttggattccgtccttcacctccagatcgcgaagaatcggttgaggagtttgttagacgtaatcttggagatgaagttttcgaacgcttgatagaacctttttgctcaggtgtttatgctggtgatccatcaaaacttagtatgaaagcagcatttgggaaggtctggaatctggagcaaaatggtggtagcattgttggtggagccttcaaggctattcaggacagaaagaatagtcaaaagcctccacgggacccgaggttaccgaaaccaaagggccaaactgttggatcttttaggaaaggacaagcgatgttgcctaatgcaatctcaacgaggttaggtagcagagtgaaattgtgttggaagctcacgagtatttcaaaattggagaatagaggttataatttgacatatgaaacaccacaaggatttgaaagtctgcagactaaaactatcgtgatgactgttccatcctacgtggcgagtgacttgttgcgtccgctttcgttgggtgcagcagatgcattgtcaaaattttattatcctccggttgcagctgtatcaatttcatatccaaaagacgcaattcgtgctgaccggctgattgatggtcaactcaaaggttttgggcaattgcatccacgaagtcaaggggtggaaactttaggtacgatctacagttcatctcttttccctaaccgagcgccacctggaagggttctgctcttgaactacatcggaggggctacaaatcctgaaattctatcaaagacggagggcgaaattgtggatgcggtggaccgggacctacggacgatgctgataaggcgtgatgcggaagatccattgacgttgggggtgcgggtgtggcctcgagcaatcccgcagtttctgatcggtcattatgacattctagattctgcaaaagctgctctgagtagcggtggattccaaggtatgtttcttggtggcaactatgtgtctggtgtggctttaggtaaatgtgtcgaggctgcttatgatgttgccgctgaggtaatgaactttttgtcgcaaggggtgtacaagtga

SEQ ID NO: 14 - protein, 555 aa, Cichorium sp.
MTSLTDVCSLNCCRSWSSLPPPVSGGSLTSKNPRYLITYSPAHRKCNRWRFRCSIAKDSPITPPISNEFNSQPLLDCVIVGAGISGLCIAQALATKHASVSPDVIVTEARDRVGGNISTVERDGYLWEEGPNSFQPSDAMLTMVVDSGLKDDLVLGDPTAPRFVLWGGDLKPVPSKPADLPFFDLMSFPGKLRAGFGALGFRPSPPDREESVEEFVRRNLGDEVFERLIEPFCSGVYAGDPSKLSMKAAFGKVWNLEQNGGSIVGGAFKAIQDRKNSQKPPRDPRLPKPKGQTVGSFRKGQAMLPNAISTRLGSRVKLCWKLTSISKLENRGYNLTYETPQGFESLQTKTIVMTVPSYVASDLLRPLSLGAADALSKFYYPPVAAVSISYPKDAIRADRLIDGQLKGFGQLHPRSQGVETLGTIYSSSLFPNRAPPGRVLLLNYIGGATNPEILSKTEGEIVDAVDRDLRTMLIRRDAEDPLTLGVRVWPRAIPQFLIGHYDILDSAKAALSSGGFQGMFLGGNYVSGVALGKCVEAAYDVAAEVMNFLSQGVYK

SEQ ID NO: 15 - DNA, 1689 bp, Spinacia oleracea
  source 1..1689: mol_type = other DNA
atgagcgctatggcgttatcgagtacaatggccctttcgttgccgcaatcttctatgtcattatcccattgtaggcacaaccgtatcaccattttgattccatcttcgtcgcttcgaagacgaggaggaagctctatccgctgctctacaatctcaacctctaattccgcggctgcagccaattaccagaacaaaaacataggcacaaacggagttgacggcggcggaggcggaggaggtgtgttagactgtgtgattgtaggaggtggaatcagtggactttgcattgcacaggctctatctactaaatactccaacctctccacgaatttcattgtcaccgaggctaaggatcgagttggcgggaacatcactaccatggaagctgatgggtatttatgggaagagggtcctaatagctttcagccatctgatgcagtgctcaccatggctgttgacagtggtttgaaagaggaattggtgctgggagatcccaattcgcctcgctttgtgctgtggaatggcaaattaaggcctgtaccttccaagctcactgacctccctttctttgatctcatgagcttccctggaaagattagggctggtcttggtgctcttggcttacgaccatctcctccggctcatgaggaatccgttgaacaatttgtccgtcgtaatcttggtgatgaggtctttgaacgcttgatcgaacctttttgttcaggtgtgtatgctggtgatccttccaagttgagtatgaaagctgcttttggcagggtttgggtcttggagcaaaagggtggtagtatcattggtggcaccctcaaaacaatccaggaaagaaaggataatcctaagccacctcgagacccgcgcctccccaaaccaaagggccagacagttggatccttcaggaaaggactgagtatgttgccaaccgccatttctgaaaggcttggcaacaaagtgaaagtatcatggaccctttctggtattgctaagtcgtcgaacggagagtataatctgacttatgaaacaccagatggactggtttccgttaggaccaaaagtgttgtgatgactgtcccgtcatatgttgcaagtagcctccttcgtccactttcagatgtcgccgcagaatctctttcaaaatttcattatccaccagttgcagctgtgtcactttcctatcctaaagaagcaattagatcagagtgcttgattgacggtgaacttaaaggattcgggcaattacattcccgcagtcaaggtgtggaaaccttgggaacaatttatagttcatctcttttccctgggcgagcaccacctggtaggaccttgattttgaactacattggaggtgatactaaccctggcatattagacaagacgaaagatgaactagctgaagcagttgacagggatttgagaagaattctcataaaccctaatgcaaaagctccccgggttttgggtgtgagagtatggccacaagcaattccccaatttttaattggccactttgatctgctcgatgcagcaaaagctgctttgactgatggtggacacaaaggattgtttcttggtggaaactatgtatcaggtgttgctttgggccgatgtatagagggtgcttatgaatctgcagccgaggttgtagattttctgtcacagtactcggataaatag

SEQ ID NO: 16 - protein, 562 aa, Spinacia sp.
MSAMALSSTMALSLPQSSMSLSHCRHNRITILIPSSSLRRRGGSSIRCSTISTSNSAAAANYQNKNIGTNGVDGGGGGGGVLDCVIVGGGISGLCIAQALSTKYSNLSTNFIVTEAKDRVGGNITTMEADGYLWEEGPNSFQPSDAVLTMAVDSGLKEELVLGDPNSPRFVLWNGKLRPVPSKLTDLPFFDLMSFPGKIRAGLGALGLRPSPPAHEESVEQFVRRNLGDEVFERLIEPFCSGVYAGDPSKLSMKAAFGRVWVLEQKGGSIIGGTLKTIQERKDNPKPPRDPRLPKPKGQTVGSFRKGLSMLPTAISERLGNKVKVSWTLSGIAKSSNGEYNLTYETPDGLVSVRTKSVVMTVPSYVASSLLRPLSDVAAESLSKFHYPPVAAVSLSYPKEAIRSECLIDGELKGFGQLHSRSQGVETLGTIYSSSLFPGRAPPGRTLILNYIGGDTNPGILDKTKDELAEAVDRDLRRILINPNAKAPRVLGVRVWPQAIPQFLIGHFDLLDAAKAALTDGGHKGLFLGGNYVSGVALGRCIEGAYESAAEVVDFLSQYSDK

SEQ ID NO: 17 - DNA, 1596 bp, Spinacia oleracea
  source 1..1596: mol_type = other DNA
atggtaatactaccggtttcccagctatcaactaatctgggtttatcgctggtttcacccaccaagaacaacccagttatgggcaacgtttctgagcgaaatcaagtcaatcaacccatttctgctaaaagggttgctgttgttggtgctggtgttagtggacttgctgcggcgtataagctaaaatcgaatggcttgaatgtgacattgtttgaagctgatagtagagctggtgggaaactcaaaactgttgtaaaggatggtttgatttgggatgaaggggcaaataccatgacagagagcgatgaggaggtcacgagtttgtttgatgatctcgggattcgtgagaagctacagctaccaatttcacaaaacaaaagatacattgccagagatggtcttcctgtgctgttaccttcaaatccagttgcgctcctgaagagcaatatcctttcagcaaaatctaagctacaaattatgttggaaccttttctttggaaaaaacacaatggtgctaaggtttctgacgagaatgcccaagaaagtgtggctgagttttttgagcggcattttgggaaagagtttgttgattatttaattgatccttttgtcgcgggtacaagtggtggagatcctcaatctctttctatgcgtcatgcatttccagaattatggaatattgagaacaggtttggttcagtgatttctggattcattcagtctaaactgtcatccaagaaggaaaagggtggagaaaagcaatcttctaataagaagccacgtgtacgtggttcgttttcttttcagggtggaatgcagacactagttgacactatatgcaaagagtttggtgaagatgaactcaaactccagtctgaggttctttcattgtcatacagccataatggaagccttacatcagagaattggtcagtgtcttctatgtcaaacagcaccatccaagatcaaccatatgatgctgtcgttgtgaccgccccaatcaataatgtcaaagaactgaagattatgaaagtggaaaacccattttctcttgacttcattccagaggtgagctgtctacccctctctgttattattactacattcaagaagaccaatgtgaagagacctcttgagggttttggtgttcttgtaccctctaatgagcaacataatgggctgaagactcttggtactttgttttcctcaatgatgtttcctgatcgtgctccctctgatgtgtatctatacactacctttgttggaggtagcagaaatagagaacttgcaaaagcttcaacggatgaactgaagcaaatagtttcttctgacctccagcagctgttgggcaccgagggcgaacctacttttgtgaatcatttttactggagcaaagcattccctctttatggacgcaattacgactcagttcttagagcaatagagaagatggaaagggaccttcctggacttttttacgcaggtaaccataagggtggactgtctgtgggaaagtcaatagcctctggatacaaagctgccgagcttgcgatatcctatctcgagtctaacaagatgaccgaggagactatataa

SEQ ID NO: 18 - protein, 531 aa, Spinacia sp.
MVILPVSQLSTNLGLSLVSPTKNNPVMGNVSERNQVNQPISAKRVAVVGAGVSGLAAAYKLKSNGLNVTLFEADSRAGGKLKTVVKDGLIWDEGANTMTESDEEVTSLFDDLGIREKLQLPISQNKRYIARDGLPVLLPSNPVALLKSNILSAKSKLQIMLEPFLWKKHNGAKVSDENAQESVAEFFERHFGKEFVDYLIDPFVAGTSGGDPQSLSMRHAFPELWNIENRFGSVISGFIQSKLSSKKEKGGEKQSSNKKPRVRGSFSFQGGMQTLVDTICKEFGEDELKLQSEVLSLSYSHNGSLTSENWSVSSMSNSTIQDQPYDAVVVTAPINNVKELKIMKVENPFSLDFIPEVSCLPLSVIITTFKKTNVKRPLEGFGVLVPSNEQHNGLKTLGTLFSSMMFPDRAPSDVYLYTTFVGGSRNRELAKASTDELKQIVSSDLQQLLGTEGEPTFVNHFYWSKAFPLYGRNYDSVLRAIEKMERDLPGLFYAGNHKGGLSVGKSIASGYKAAELAISYLESNKMTEETI

SEQ ID NO: 19 - DNA, 1674 bp, Solanum tuberosum
  source 1..1674: mol_type = other DNA
atgacaacaacggccgtcgccaaccatcctagcattttcactcaccggtcgccgctgccgtcgccgtcgtcctcctcctcatcgccgtcatttttatttttaaaccgtacgaatttcattccttacttttccacctccaagcgcaatagtgtcaattgcaatggctggagaacacgatgttccgttgccaaggattatacagttcctccctcggaagtcgacggtaatcagttcccggagctggattgtgtggtagttggagcaggaattagtggactctgcattgctaaggtgatttcggctaattatcccaatttgatggtgacggaggcgagggatcgtgccggtggaaacataacgacggtggaaagagatggatacttatgggaagaaggtcctaacagtttccagccttcggatcctatgttgacaatggctgtagattgtggattgaaggatgatttggtgttgggagatcctgatgcgcctcgctttgtcttgtggaaggataaactaaggcctgttcccggcaagctcactgatcttcccttctttgatttgatgagtatccctggcaagctcagagctggttttggtgccattggccttcgcccttcacctccaggttatgaggaatcagttgagcagttcgtgcgtcgtaatcttggtgcagaagtctttgaacgtttgattgaaccattttgttctggtgtttacgccggtgacccctcaaaattgattatgaaagcagcatttgggaaagtgtggaagctagaacaaactggtggtagcattattgggggaacctttaaagcaattaaggagagatccagtaaccctaaaccgcctcgtgatccgcgtttaccaacaccaaaaggacaaactgttggatcatttaggaagggtctgagaatgctgccggatgcaatttgtgaaagactgggaagcaaagtaaaactatcatggaagctttctagcattacaaagtcagaaaaaggaggatatctcttgacatacgagacaccagaaggagtagtttctctgcgaagtcgaagcattgtcatgactgttccatcctatgtagcaagcaacatattacgccctctttcggtcgctgcagcagatgcactttcaagtttctactatcccccagtagcagcagtgacaatttcatatcctcaagaggctattcgtgatgagcgtctggttgatggtgaactaaagggatttgggcagttgcatccacgttcacagggagtggaaacactaggaacaatatatagttcatcactctttcctaaccgtgctccaaatggccgggtgctactcttgaactacattggaggagcaacaaatactgaaattgtgtctaagacggagagccaacttgtggaagcagttgaccgtgacctcagaaaaatgcttataaaacccaaagcacaagatccctttgttacgggtgtgcgagtatggccacaagctatcccacagtttttggtcggacatctggatacactaggtactgcaaaaactgctctaagtgataatgggcttgacgggctattccttgggggtaattatgtgtctggtgtagcattgggaaggtgtgttgaaggtgcttatgaaatagcatctgaggtaactggatttctgtctcagtatgcatacaaatga

SEQ ID NO: 20 - protein, 557 aa, Solanum tuberosum
MTTTAVANHPSIFTHRSPLPSPSSSSSSPSFLFLNRTNFIPYFSTSKRNSVNCNGWRTRCSVAKDYTVPPSEVDGNQFPELDCVVVGAGISGLCIAKVISANYPNLMVTEARDRAGGNITTVERDGYLWEEGPNSFQPSDPMLTMAVDCGLKDDLVLGDPDAPRFVLWKDKLRPVPGKLTDLPFFDLMSIPGKLRAGFGAIGLRPSPPGYEESVEQFVRRNLGAEVFERLIEPFCSGVYAGDPSKLIMKAAFGKVWKLEQTGGSIIGGTFKAIKERSSNPKPPRDPRLPTPKGQTVGSFRKGLRMLPDAICERLGSKVKLSWKLSSITKSEKGGYLLTYETPEGVVSLRSRSIVMTVPSYVASNILRPLSVAAADALSSFYYPPVAAVTISYPQEAIRDERLVDGELKGFGQLHPRSQGVETLGTIYSSSLFPNRAPNGRVLLLNYIGGATNTEIVSKTESQLVEAVDRDLRKMLIKPKAQDPFVTGVRVWPQAIPQFLVGHLDTLGTAKTALSDNGLDGLFLGGNYVSGVALGRCVEGAYEIASEVTGFLSQYAYK

SEQ ID NO: 21 - DNA, 1608 bp, Zea mays
  source 1..1608: mol_type = other DNA
atggtcgccgccacagccaccgccatggccaccgctgcatcgccgctactcaacgggacccgaatacctgcgcggctccgccatcgaggactcagcgtgcgctgcgctgctgtggcgggcggcgcggccgaggcaccggcatccaccggcgcgcggctgtccgcggactgcgtcgtggtgggcggaggcatcagtggcctctgcaccgcgcaggcgctggccacgcggcacggcgtcggggacgtgcttgtcacggaggcccgcgcccgccccggcggcaacattaccaccgtcgagcgccccgaggaagggtacctctgggaggagggtcccaacagcttccagccctccgaccccgttctcaccatggccgtggacagcggactgaaggatgacttggtttttggggacccaaacgcgccgcgtttcgtgctgtgggaggggaagctgaggcccgtgccatccaagcccgccgacctcccgttcttcgatctcatgagcatcccagggaagctcagggccggtctaggcgcgcttggcatccgcccgcctcctccaggccgcgaagagtcagtggaggagttcgtgcgccgcaacctcggtgctgaggtctttgagcgcctcattgagcctttctgctcaggtgtctatgctggtgatccttctaagctcagcatgaaggctgcatttgggaaggtttggcggttggaagaaactggaggtagtattattggtggaaccatcaagacaattcaggagaggagcaagaatccaaaaccaccgagggatgcccgccttccgaagccaaaagggcagacagttgcatctttcaggaagggtcttgccatgcttccaaatgccattacatccagcttgggtagtaaagtcaaactatcatggaaactcacgagcattacaaaatcagatgacaagggatatgttttggagtatgaaacgccagaaggggttgtttcggtgcaggctaaaagtgttatcatgactattccatcatatgttgctagcaacattttgcgtccactttcaagcgatgctgcagatgctctatcaagattctattatccaccggttgctgctgtaactgtttcgtatccaaaggaagcaattagaaaagaatgcttaattgatggggaactccagggctttggccagttgcatccacgtagtcaaggagttgagacattaggaacaatatacagttcctcactctttccaaatcgtgctcctgacggtagggtgttacttctaaactacataggaggtgctacaaacacaggaattgtttccaagactgaaagtgagctggtcgaagcagttgaccgtgacctccgaaaaatgcttataaattctacagcagtggaccctttagtccttggtgttcgagtttggccacaagccatacctcagttcctggtaggacatcttgatcttctggaagccgcaaaagctgccctggaccgaggtggctacgatgggctgttcctaggagggaactatgttgcaggagttgccctgggcagatgcgttgagggcgcgtatgaaagtgcctcgcaaatatctgacttcttgaccaagtatgcctacaagtga

SEQ ID NO: 22 - protein, 535 aa, Zea mays
MVAATATAMATAASPLLNGTRIPARLRHRGLSVRCAAVAGGAAEAPASTGARLSADCVVVGGGISGLCTAQALATRHGVGDVLVTEARARPGGNITTVERPEEGYLWEEGPNSFQPSDPVLTMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEETGGSIIGGTIKTIQERSKNPKPPRDARLPKPKGQTVASFRKGLAMLPNAITSSLGSKVKLSWKLTSITKSDDKGYVLEYETPEGVVSVQAKSVIMTIPSYVASNILRPLSSDAADALSRFYYPPVAAVTVSYPKEAIRKECLIDGELQGFGQLHPRSQGVETLGTIYSSSLFPNRAPDGRVLLLNYIGGATNTGIVSKTESELVEAVDRDLRKMLINSTAVDPLVLGVRVWPQAIPQFLVGHLDLLEAAKAALDRGGYDGLFLGGNYVAGVALGRCVEGAYESASQISDFLTKYAYK

SEQ ID NO: 23 - DNA, 1635 bp, Zea mays
  source 1..1635: mol_type = other DNA
atgctcgctttgactgcctcagcctcatccgcttcgtcccatccttatcgccacgcctccgcgcacactcgtcgcccccgcctacgtgcggtcctcgcgatggcgggctccgacgacccccgtgcagcgcccgccagatcggtcgccgtcgtcggcgccggggtcagcgggctcgcggcggcgtacaggctcagacagagcggcgtgaacgtaacggtgttcgaagcggccgacagggcgggaggaaagatacggaccaattccgagggcgggtttgtctgggatgaaggagctaacaccatgacagaaggtgaatgggaggccagtagactgattgatgatcttggtctacaagacaaacagcagtatcctaactcccaacacaagcgttacattgtcaaagatggagcaccagcactgattccttcggatcccatttcgctaatgaaaagcagtgttctttcgacaaaatcaaagattgcgttattttttgaaccatttctctacaagaaagctaacacaagaaactctggaaaagtgtctgaggagcacttgagtgagagtgttgggagcttctgtgaacgccactttggaagagaagttgttgactattttgttgatccatttgtagctggaacaagtgcaggagatccagagtcactatctattcgtcatgcattcccagcattgtggaatttggaaagaaagtatggttcagttattgttggtgccatcttgtctaagctagcagctaaaggtgatccagtaaagacaagacatgattcatcagggaaaagaaggaatagacgagtgtcgttttcatttcatggtggaatgcagtcactaataaatgcacttcacaatgaagttggagatgataatgtgaagcttggtacagaagtgttgtcattggcatgtacatttgatggagttcctgcactaggcaggtggtcaatttctgttgattcgaaggatagcggtgacaaggaccttgctagtaaccaaacctttgatgctgttataatgacagctccattgtcaaatgtccggaggatgaagttcaccaaaggtggagctccggttgttcttgactttcttcctaagatggattatctaccactatctctcatggtgactgcttttaagaaggatgatgtcaagaaacctctggaaggatttggggtcttaataccttacaaggaacagcaaaaacatggtctgaaaacccttgggactctcttttcctcaatgatgttcccagatcgagctcctgatgaccaatatttatatacaacatttgttgggggtagccacaatagagatcttgctggagctccaacgtctattctgaaacaacttgtgacctctgaccttaaaaaactcttgggcgtagaggggcaaccaacttttgtcaagcatgtatactggggaaatgcttttcctttgtatggccatgattatagttctgtattggaagctatagaaaagatggagaaaaaccttccagggttcttctacgcaggaaatagcaaggatgggcttgctgttggaagtgttatagcttcaggaagcaaggctgctgaccttgcaatctcatatcttgaatctcacaccaagcataataattcacattga

SEQ ID NO: 24 - protein, 544 aa, Zea mays
MLALTASASSASSHPYRHASAHTRRPRLRAVLAMAGSDDPRAAPARSVAVVGAGVSGLAAAYRLRQSGVNVTVFEAADRAGGKIRTNSEGGFVWDEGANTMTEGEWEASRLIDDLGLQDKQQYPNSQHKRYIVKDGAPALIPSDPISLMKSSVLSTKSKIALFFEPFLYKKANTRNSGKVSEEHLSESVGSFCERHFGREVVDYFVDPFVAGTSAGDPESLSIRHAFPALWNLERKYGSVIVGAILSKLAAKGDPVKTRHDSSGKRRNRRVSFSFHGGMQSLINALHNEVGDDNVKLGTEVLSLACTFDGVPALGRWSISVDSKDSGDKDLASNQTFDAVIMTAPLSNVRRMKFTKGGAPVVLDFLPKMDYLPLSLMVTAFKKDDVKKPLEGFGVLIPYKEQQKHGLKTLGTLFSSMMFPDRAPDDQYLYTTFVGGSHNRDLAGAPTSILKQLVTSDLKKLLGVEGQPTFVKHVYWGNAFPLYGHDYSSVLEAIEKMEKNLPGFFYAGNSKDGLAVGSVIASGSKAADLAISYLESHTKHNNSH

SEQ ID NO: 25 - DNA, 1692 bp, Chlamydomonas reinhardtii
  source 1..1692: mol_type = other DNA
atgatgttgacccagactcctgggaccgccacggcttctagccggcggtcgcagatccgctcggctgcgcacgtctccgccaaggtcgcgcctcggcccacgccattctcggtcgcgagccccgcgaccgctgcgagccccgcgaccgcggcggcccgccgcacactccaccgcactgctgcggcggccactggtgctcccacggcgtccggagccggcgtcgccaagacgctcgacaatgtgtatgacgtgatcgtggtcggtggaggtctctcgggcctggtgaccggccaggccctggcggctcagcacaaaattcagaacttccttgttacggaggctcgcgagcgcgtcggcggcaacattacgtccatgtcgggcgatggctacgtgtgggaggagggcccgaacagcttccagcccaacgatagcatgctgcagattgcggtggactctggctgcgagaaggaccttgtgttcggtgaccccacggctccccgcttcgtgtggtgggagggcaagctgcgccccgtgccctcgggcctggacgccttcaccttcgacctcatgtccatccccggcaagatccgcgccgggctgggcgccatcggcctcatcaacggagccatgccctccttcgaggagagtgtggagcagttcatccgccgcaacctgggcgatgaggtgttcttccgcctgatcgagcccttctgctccggcgtgtacgcgggcgacccctccaagctgtccatgaaggcggccttcaacaggatctggattctggagaagaacggcggcagcctggtgggaggtgccatcaagctgttccaggaacgccagtccaacccggccccgccgcgggacccgcgcctgccgcccaagcccaagggccagacggtgggctcgttccgcaagggcctgaagatgctgccggacgccattgagcgcaacatccccgacaagatccgcgtgaactggaagctggtgtctctgggccgcgaggcggacgggcggtacgggctggtgtacgacacgcccgagggccgtgtcaaggtgtttgcccgcgccgtggctctgaccgcgcccagctacgtggtggcggacctggtcaaggagcaggcgcccgccgccgccgaggccctgggctccttcgactacccgccggtgggcgccgtgacgctgtcgtacccgctgagcgccgtgcgggaggagcgcaaggcctcggacgggtccgtgccgggcttcggtcagctgcacccgcgcacgcagggcatcaccactctgggcaccatctacagctccagcctgttccccggccgcgcgcccgagggccacatgctgctgctcaactacatcggcggcaccaccaaccgcggcatcgtcaaccagaccaccgagcagctggtggagcaggtggacaaggacctgcgcaacatggtcatcaagcccgacgcgcccaagccccgtgtggtgggcgtgcgcgtgtggccgcgcgccatcccgcagttcaacctgggccacctggagcagctggacaaggcgcgcaaggcgctggacgcggcggggctgcagggcgtgcacctggggggcaactacgtcagcggtgtggccctgggcaaggtggtggagcacggctacgagtccgcagccaacctggccaagagcgtgtccaaggccgcagtcaaggcctaa

SEQ ID NO: 26 - protein, 563 aa, Chlamydomonas sp.
MMLTQTPGTATASSRRSQIRSAAHVSAKVAPRPTPFSVASPATAASPATAAARRTLHRTAAAATGAPTASGAGVAKTLDNVYDVIVVGGGLSGLVTGQALAAQHKIQNFLVTEARERVGGNITSMSGDGYVWEEGPNSFQPNDSMLQIAVDSGCEKDLVFGDPTAPRFVWWEGKLRPVPSGLDAFTFDLMSIPGKIRAGLGAIGLINGAMPSFEESVEQFIRRNLGDEVFFRLIEPFCSGVYAGDPSKLSMKAAFNRIWILEKNGGSLVGGAIKLFQERQSNPAPPRDPRLPPKPKGQTVGSFRKGLKMLPDAIERNIPDKIRVNWKLVSLGREADGRYGLVYDTPEGRVKVFARAVALTAPSYVVADLVKEQAPAAAEALGSFDYPPVGAVTLSYPLSAVREERKASDGSVPGFGQLHPRTQGITTLGTIYSSSLFPGRAPEGHMLLLNYIGGTTNRGIVNQTTEQLVEQVDKDLRNMVIKPDAPKPRVVGVRVWPRAIPQFNLGHLEQLDKARKALDAAGLQGVHLGGNYVSGVALGKVVEHGYESAANLAKSVSKAAVKA

SEQ ID NO: 27 - DNA, 1734 bp, Polytomella sp.
  source 1..1734: mol_type = other DNA
atgtcgagttccgcactaaggctattatgcgggcgaacaagtttctttaatttatgccaaaaatatcctccttcctttctgtcacaattgtcgaccttaaatttctcaacccattcgcctttcgatagcacttatgatgtcgtcgtcgttggtgccggaatctctgggttgtctactgcccaagcacttagcattcaacataagatcgataatgttctggttactgaagctgatcatcgtgtaggcggtaaaattacgacgaaaaggaataaagatttcctgtgggaggagggtccaaatagttgcctaatgaacgacgctttatatcgcgctgcccgagatgccggcgtggaatccaaaattctatcggcggatccaaaattaccacgttggattctgtggggtcgtcgtttgcgtgtggcccccattggaagctacgctttaaaatccgaccttttatctacccaaggcctactccgtgccatccgaggagtcacaggttttggtgtgtcaccggctccacctaagggtcaggaggagagcgtggagggctttgttcgacggaccttaggagacgagatttttgagcgactcgttgagcccttttgctccggggtttatgcgggggatcctagcaaattgtccatgcgtgctgctttcggaaaacttgtggaattcgaagagacgggtgatggtagcttacttcgcggcgtctttcgttacgtaatgaacaaacgacgcgaaagaaggacgggcggggcgaaagacggggacacggtccctttgaacgagacggccaaggcacccaaatcatcctctggcccaacagtatcgtctttcgaggggggaatcgagatcctgcccaaggccattgcgcaaaagctgggtgatcgagttcgtcttggcctacgactcgtgcgcatcgatcccacgcagctcgcggatggtacgacagcgtaccgtctgtcgtaccgtcggatgagtcatcaaggcgatgacgactcgagtcgtacggcaggtgctgtaccgcgtacggcggagggggatgtcgcggcgggggacgaggacgccgtggtggaggtggtggcgaagaaggtcgtgctgacgacgccggcattcgacgccgcggacatcttgtcgcgttccggcttggtggcggcggcgaacccgttgaaggaggtggattacccgccagtagcgttggtcgttctttcgtacgacgtcgactcgatttccgccatacaccgcgtgagtcacgtggctcatggcctcagcggctttggccaactccaccctcgcccagagggtctccgtacattaggaaccatttacggcagtacattatttcccaaccgttcccccgtagctcgtacgacgcttttaaatttcgttggtggatccaccgaccgtgcagtggggtccgcggatccaatggctttggcgatggaggtggatctggatctgaaaaagagcgggttgatccgagagggagctgcgaagccagaagtcctcggggtgaaagtatatccaaaggctattcctcagtttgatattggtcatttggatcgagtggaaaaggccaaaatgatgttaaagaacgaaagggggggtgcagattggagtggggtcaaattggcgggaaattatgtgtgcggcgtcgcagtgggcagatgcatagaatttggattcgaaattgcggagaacttggcgcaggaattggcgagaaaaaaatag

SEQ ID NO: 28 - protein, 577 aa, Polytomella sp.
MSSSALRLLCGRTSFFNLCQKYPPSFLSQLSTLNFSTHSPFDSTYDVVVVGAGISGLSTAQALSIQHKIDNVLVTEADHRVGGKITTKRNKDFLWEEGPNSCLMNDALYRAARDAGVESKILSADPKLPRWILWGRRLRVAPIGSYALKSDLLSTQGLLRAIRGVTGFGVSPAPPKGQEESVEGFVRRTLGDEIFERLVEPFCSGVYAGDPSKLSMRAAFGKLVEFEETGDGSLLRGVFRYVMNKRRERRTGGAKDGDTVPLNETAKAPKSSSGPTVSSFEGGIEILPKAIAQKLGDRVRLGLRLVRIDPTQLADGTTAYRLSYRRMSHQGDDDSSRTAGAVPRTAEGDVAAGDEDAVVEVVAKKVVLTTPAFDAADILSRSGLVAAANPLKEVDYPPVALVVLSYDVDSISAIHRVSHVAHGLSGFGQLHPRPEGLRTLGTIYGSTLFPNRSPVARTTLLNFVGGSTDRAVGSADPMALAMEVDLDLKKSGLIREGAAKPEVLGVKVYPKAIPQFDIGHLDRVEKAKMMLKNERGGADWSGVKLAGNYVCGVAVGRCIEFGFEIAENLAQELARKK

SEQ ID NO: 29 - DNA, 1635 bp, Sorghum bicolor
  source 1..1635: mol_type = other DNA
atgctcgctcggactgccacggtctcctccacttcgtcccactcccatccttatcgccccacctccgctcgcagtctccgcctacgtccggtcctcgcgatggcgggctccgacgactcccgcgcagctcccgccaggtcggtcgccgtcgtcggcgccggggtcagcgggctcgtggcggcgtacaggctcaggaagagcggcgtgaatgtgacggtgttcgaggcggccgacagggcgggaggaaagatacggaccaattccgagggcgggtttctctgggatgaaggagcgaacaccatgacagaaggtgaattggaggccagtagactgatagatgatctcggtctacaagacaaacagcagtatcctaactcccaacacaagcgttacattgtcaaagatggagcaccagcactgattccttcggatcccatttcgctgatgaaaagcagtgttctttctacaaaatcaaagattgcgttattttttgaaccatttctctacaagaaagctaacacaagaaaccctggaaaagtatctgatgagcatttgagtgagagtgttgggagcttctttgaacgccacttcggaagagaagttgttgactatcttattgatccatttgtagctggaacaagtgcaggagatccagagtcactatctatttgtcatgcattcccagcactgtggaatttggaaagaaaatatggttcagttgttgttggtgccatcttgtctaagctaacagctaaaggtgatccagtaaagacaagacgtgattcatcagcgaaaagaaggaatagacgcgtgtcgttttcatttcatggtggaatgcagtcactaataaatgcacttcacaatgaagttggagatgataatgtgaagcttggtacagaagtgttgtcattggcgtgtacattagatggagcccctgcaccaggcgggtggtcaatttctgatgattcgaaggatgctagtggcaaggaccttgctaaaaaccaaacctttgatgctgttataatgacagctccattgtcaaatgtccagaggatgaagttcacaaaaggtggagctccttttgttctagactttcttcctaaggtggattatctaccactatctctcatggtgactgcttttaagaaggaagatgtcaagaaacctctggaaggatttggcgtcttaataccctacaaggaacagcaaaaacatggtctaaaaacccttgggactctcttctcctcaatgatgttcccagatcgagctcctgacgaccaatatttatatacaacatttgttgggggtagccacaatagagatcttgctggagctccaacgtctattctgaaacaacttgtgacctctgaccttaaaaaactcttaggcgtacaggggcaaccaacttttgtcaagcatatatactggggaaatgcttttcctttgtatggtcatgattacaattctgtattggaagctatagaaaagatggagaaaaatcttccagggttcttctacgcaggaaataacaaggatgggcttgctgttgggagtgttatagcttcaggaagcaaggctgctgaccttgcaatctcgtatcttgaatctcacaccaagcataataatttacattga

SEQ ID NO: 30 - protein, 544 aa, Sorghum sp.
MLARTATVSSTSSHSHPYRPTSARSLRLRPVLAMAGSDDSRAAPARSVAVVGAGVSGLVAAYRLRKSGVNVTVFEAADRAGGKIRTNSEGGFLWDEGANTMTEGELEASRLIDDLGLQDKQQYPNSQHKRYIVKDGAPALIPSDPISLMKSSVLSTKSKIALFFEPFLYKKANTRNPGKVSDEHLSESVGSFFERHFGREVVDYLIDPFVAGTSAGDPESLSICHAFPALWNLERKYGSVVVGAILSKLTAKGDPVKTRRDSSAKRRNRRVSFSFHGGMQSLINALHNEVGDDNVKLGTEVLSLACTLDGAPAPGGWSISDDSKDASGKDLAKNQTFDAVIMTAPLSNVQRMKFTKGGAPFVLDFLPKVDYLPLSLMVTAFKKEDVKKPLEGFGVLIPYKEQQKHGLKTLGTLFSSMMFPDRAPDDQYLYTTFVGGSHNRDLAGAPTSILKQLVTSDLKKLLGVQGQPTFVKHIYWGNAFPLYGHDYNSVLEAIEKMEKNLPGFFYAGNNKDGLAVGSVIASGSKAADLAISYLESHTKHNNLH

SEQ ID NO: 31 - DNA, 1017 bp, Chlorella sp.
  source 1..1017: mol_type = other DNA
atggcctccacagcaacactgcacggcgcgccctgctgctcggcgcggcccgtgggccgccggcatattgcagcaccgagcatccagcacaatgggccgcgcctggcggccagggtgcagcagcggaagggggcaggggagcggcgctcggcactgcgtgtgcaggccgtccaggcccctcccgagaaggcgggggcgagcacagggagcgcagcagacgacagcggcgtttacgacgttgtggtcgtgggcgccggcatctccggcctcaccaccgcccaggcgctgaccacgcagcacagcggcgtggcgcggcgggtgctggtgaccgagggccgcgaccgcgtgggcggcaacatcacctccgtgtccaacaaggaggaggggctgctgtgggaggaggggcccaactccttccagccaaacgactccatcctgcaggccgcggtggacgccggcgtggcggaccagctggtactgggcgaccccacggcgccgcgttttgtgtactgggacaagaagctgcgccccacgccctccggccccgacgcgctcacgttcgacctgatgagcatcgtgggcaagatccgggcggggctgggcgcgctgggcttcaaggcgcccatgccagactatgaggagagcgtggagcagtatgtgcggcgcaacctgggggccgaggtgtttgagcgcctgatcgagcccttctgcagcggcgtgtacgccggcgaccccaagaagctgtccatgaaggcggcctttggcaaggtgtacgacctggagaagaagggcggcagcatcgtgggcggcgtgatcaagctgattcaggagcggcgcgccaacccgccgccgccgcgcagcccagcgctgccgcccaagcccgcgggccagacggtgggctccttccgctccggcctgcgcacgctgccggatgccatggcggcgcggctgggagacgcggtgcgcaccagctggcagctcaaggagctcagcaaggaaggggaggcctacaagtga

SEQ ID NO: 32 - protein, 338 aa, Chlorella sp.
MASTATLHGAPCCSARPVGRRHIAAPSIQHNGPRLAARVQQRKGAGERRSALRVQAVQAPPEKAGASTGSAADDSGVYDVVVVGAGISGLTTAQALTTQHSGVARRVLVTEGRDRVGGNITSVSNKEEGLLWEEGPNSFQPNDSILQAAVDAGVADQLVLGDPTAPRFVYWDKKLRPTPSGPDALTFDLMSIVGKIRAGLGALGFKAPMPDYEESVEQYVRRNLGAEVFERLIEPFCSGVYAGDPKKLSMKAAFGKVYDLEKKGGSIVGGVIKLIQERRANPPPPRSPALPPKPAGQTVGSFRSGLRTLPDAMAARLGDAVRTSWQLKELSKEGEAYK

SEQ ID NO: 33 - DNA, 1611 bp, Oryza sativa
  source 1..1611: mol_type = other DNA
atggccgccgccgccgcagccatggccaccgccacctccgccacggcagcgccgccgctccgcattcgcgacgccgcgaggaggacccgccgacgcggccacgttcgctgcgccgtcgccagcggcgcggccgaggcgcccgcggcgcccggggcgcgggtgtcggcggactgcgtcgtggtgggcggcggcatcagcgggctctgcaccgcgcaggcgctggccacaaagcacggcgtcggcgacgtgctcgtcacggaggcccgcgcccgccccggcggcaacatcaccaccgccgagcgcgccggcgagggctacctctgggaggaggggcccaacagcttccagccttccgaccccgtcctcaccatggccgtggacagcgggctcaaggacgatctcgtgttcggggaccccaacgcgccgcggttcgtgctgtgggaggggaagctaaggccggtgccgtccaagcccggcgacctgccgttcttcgacctcatgagcatccccggcaagctcagggccggccttggcgcgctcggcgttcgagcgccacctccagggcgtgaggagtcggtggaggacttcgtgcggcgcaacctcggcgcggaggtctttgagcgcctcattgagcctttctgctcaggtgtgtatgctggtgatccttcaaagctcagtatgaaggctgcatttgggaaggtgtggaggctggaggatactggaggtagcattattggtggaaccatcaaaacaatccaggagagggggaaaaaccccaaaccgccgagggatccccgccttccaacgccaaaggggcagacagttgcatctttcaggaagggtctgactatgctcccggatgctattacatctaggttgggtagcaaagtcaaactttcatggaagttgacaagcattacaaagtcagacaacaaaggatatgcattagtgtatgaaacaccagaaggggtggtctcggtgcaagctaaaactgttgtcatgaccatcccatcatatgttgctagtgatatcttgcggccactttcaagtgatgcagcagatgctctgtcaatattctattatccaccagttgctgctgtaactgtttcatatccaaaagaagcaattagaaaagaatgcttaattgacggagagctccagggtttcggccagctgcatccgcgtagtcagggagttgagactttaggaacaatatatagctcatcactctttccaaatcgtgctccagctggaagggtgttacttctgaactacataggaggttctacaaatacagggattgtttccaagactgaaagtgagctggtagaagcagttgaccgtgacctcaggaagatgctgataaatcctaaagcagtggaccctttggtccttggcgtccgggtatggccacaagccataccacagttcctcattggccatcttgatcatcttgaggctgcaaaatctgccctgggcaaaggtggttatgatggattgttcctcggagggaactatgttgcaggagttgccctgggccgatgcgttgaaggtgcatatgagagtgcctcacaaatatctgactacttgaccaagtacgcctacaagtga

SEQ ID NO: 34 - protein, 536 aa, Oryza sativa
MAAAAAAMATATSATAAPPLRIRDAARRTRRRGHVRCAVASGAAEAPAAPGARVSADCVVVGGGISGLCTAQALATKHGVGDVLVTEARARPGGNITTAERAGEGYLWEEGPNSFQPSDPVLTMAVDSGLKDDLVFGDPNAPRFVLWEGKLRPVPSKPGDLPFFDLMSIPGKLRAGLGALGVRAPPPGREESVEDFVRRNLGAEVFERLIEPFCSGVYAGDPSKLSMKAAFGKVWRLEDTGGSIIGGTIKTIQERGKNPKPPRDPRLPTPKGQTVASFRKGLTMLPDAITSRLGSKVKLSWKLTSITKSDNKGYALVYETPEGVVSVQAKTVVMTIPSYVASDILRPLSSDAADALSIFYYPPVAAVTVSYPKEAIRKECLIDGELQGFGQLHPRSQGVETLGTIYSSSLFPNRAPAGRVLLLNYIGGSTNTGIVSKTESELVEAVDRDLRKMLINPKAVDPLVLGVRVWPQAIPQFLIGHLDHLEAAKSALGKGGYDGLFLGGNYVAGVALGRCVEGAYESASQISDYLTKYAYK

SEQ ID NO: 35 - DNA, 1518 bp, Amaranthus tuberculatus
  source 1..1518: mol_type = other DNA
atgggcaacatttctgagcgggatgaacccacttctgctaaaagggttgctgttgttggtgctggagttagtggacttgctgctgcatataagctaaaatcccatggtttgaatgtgacattgtttgaagctgattctagagctggaggcaaacttaaaactgttaaaaaagatggttttatttgggatgagggggcaaatactatgacagaaagtgaggcagaagtctcgagtttgatcgatgatcttgggcttcgtgagaagcaacagttgccaatttcacaaaataaaagatacatagctagagatggtcttcctgtgctactaccttcaaatcccgctgcactgctcacgagcaatatcctttcagcaaaatcaaagctgcaaattatgttggaaccatttttctggagaaaacacaatgctactgagctttctgatgagcatgttcaggaaagcgttggtgaattttttgagcgacattttgggaaagagtttgttgattatgttattgacccttttgttgcgggtacatgtggtggagatcctcaatcgctttctatgcaccatacatttccagaagtatggaatattgaaaaaaggtttggctctgtgtttgctggactaattcaatcaacattgttatctaagaaggaaaagggtggaggaggaaatgcttctatcaagaagcctcgtgtacgtggttcattttcattccatggtggaatgcagacacttgttgacacaatatgcaaacagcttggtgaagatgaactcaaactccagtgtgaggtgctgtccttgtcatacaaccagaaggggatcccttcattagggaattggtcagtctcttctatgtcaaataataccagtgaagatcaatcttatgatgctgtggttgtcactgctccaattcgcaatgtcaaagaaatgaagattatgaaattcggaaatccattttcacttgactttattccagaggtgagttacgtacccctctctgttatgattactgcattcaagaaggataaagtgaagagaccactcgagggctttggagttcttatcccctctaaagagcaacataatggactgaagactcttggtactttattttcctccatgatgtttcccgatcgtgctccatctgacatgtgtctctttactacatttgtcggaggaagcagaaatagaaaacttgcaaacgcttcaacggatgaattgaagcaaatagtttcttctgaccttcagcagctgttgggcactgaggacgaaccttcatttgtcaatcatctcttttggagcaacgcattcccgttgtatggacacaattacgattctgttttgagagccatagacaagatggaaaaggatcttcctggatttttttatgcaggtaaccataagggtggactttcagtgggaaaagcgatggcctccggatgcaaggctgcggaacttgtaatatcctatctggactctcatatatatgtgaagatggatgagaagaccgcgtaa

SEQ ID NO: 36 - protein, 505 aa, Amaranthus sp.
MGNISERDEPTSAKRVAVVGAGVSGLAAAYKLKSHGLNVTLFEADSRAGGKLKTVKKDGFIWDEGANTMTESEAEVSSLIDDLGLREKQQLPISQNKRYIARDGLPVLLPSNPAALLTSNILSAKSKLQIMLEPFFWRKHNATELSDEHVQESVGEFFERHFGKEFVDYVIDPFVAGTCGGDPQSLSMHHTFPEVWNIEKRFGSVFAGLIQSTLLSKKEKGGGGNASIKKPRVRGSFSFHGGMQTLVDTICKQLGEDELKLQCEVLSLSYNQKGIPSLGNWSVSSMSNNTSEDQSYDAVVVTAPIRNVKEMKIMKFGNPFSLDFIPEVSYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 37 - DNA, 1521 bp, Arabidopsis thaliana
  source 1..1521: mol_type = other DNA
atggagttatctcttctccgtccgacgactcaatcgcttcttccgtcgttttcgaagcccaatctccgattaaatgtttataagcctcttagactccgttgttcagtggccggtggaccaaccgtcggatcttcaaaaatcgaaggcggaggaggcaccaccatcacgacggattgtgtgattgtcggcggaggtattagtggtctttgcatcgctcaggcgcttgctactaagcatcctgatgctgctccgaatttaattgtgaccgaggctaaggatcgtgttggaggcaacattatcactcgtgaagagaatggttttctctgggaagaaggtcccaatagttttcaaccgtctgatcctatgctcactatggtggtagatagtggtttgaaggatgatttggtgttgggagatcctactgcgccaaggtttgtgttgtggaatgggaaattgaggccggttccatcgaagctaacagacttaccgttctttgatttgatgagtattggtgggaagattagagctggttttggtgcacttggcattcgaccgtcacctccaggtcgtgaagaatctgtggaggagtttgtacggcgtaacctcggtgatgaggtttttgagcgcctgattgaaccgttttgttcaggtgtttatgctggtgatccttcaaaactgagcatgaaagcagcgtttgggaaggtttggaaactagagcaaaatggtggaagcataataggtggtacttttaaggcaattcaggagaggaaaaacgctcccaaggcagaacgagacccgcgcctgccaaaaccacagggccaaacagttggttctttcaggaagggacttcgaatgttgccagaagcaatatctgcaagattaggtagcaaagttaagttgtcttggaagctctcaggtatcactaagctggagagcggaggatacaacttaacatatgagactccagatggtttagtttccgtgcagagcaaaagtgttgtaatgacggtgccatctcatgttgcaagtggtctcttgcgccctctttctgaatctgctgcaaatgcactctcaaaactatattacccaccagttgcagcagtatctatctcgtacccgaaagaagcaatccgaacagaatgtttgatagatggtgaactaaagggttttgggcaattgcatccacgcacgcaaggagttgaaacattaggaactatctacagctcctcactctttccaaatcgcgcaccgcccggaagaattttgctgttgaactacattggcgggtctacaaacaccggaattctgtccaagtctgaaggtgagttagtggaagcatttctagttggtcactttgatatccttgacacggctaaatcatctctaacgtcttcgggctacgaagggctatttttgggtggcaattacgtcgctggtgtagccttaggccggtgtgtagaaggcgcatatgaaaccgcgattgaggtcaacaacttcatgtcacggtacgcttacaagtaa

SEQ ID NO: 38 - protein, 506 aa, Arabidopsis sp.
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAFLVGHFDILDTAKSSLTSSGYEGLFLGGNYVAGVALGRCVEGAYETAIEVNNFMSRYAYK

SEQ ID NO: 39 - DNA, 1515 bp, Nicotiana tabacum
  source 1..1515: mol_type = other DNA
atggctccttctgccggagaagataaacacagttctgcgaagagagtcgcagtcattggtgcaggcgtcagtgggcttgctgcagcatacaagttgaaaatccatggcttgaatgtgacagtatttgaagcagaagggaaagctggagggaagttacgtagcgtgagccaagatggcctgatatgggatgaaggggcaaatactatgactgaaagtgaaggtgatgttacatttttgattgattctcttggactccgagaaaagcaacaatttccactttcacaaaacaagcgctacattgccagaaatggtactcctgtactgttaccttcaaatccaattgatctgatcaaaagcaattttctttccactggatcaaagcttcagatgcttctggaaccaatattatggaagaataaaaagctctcccaggtgtctgactcacatgaaagtgtcagtggattcttccagcgtcattttggaaaggaggttgttgactatctaattgacccttttgttgctggaacgtgtggtggtgatcctgactcgctttcaatgcaccattcatttccagagttgtggaatttagagaaaaggtttggctcagtcatacttggagctattcgatctaagttatcccctaaaaatgaaaagaagcaagggccacccaaaacttcagcaaataagaagcgccagcggggatctttttcctttttgggcggaatgcaaacacttactgatgcaatatgcaaagatctcagagaagatgaacttagactaaactctagagttctggaattatcttgtagctgtactgaggactctgcgatagatagctggtcaattatttctgcctctccacacaaaaggcaatcagaagaagaatcatttgatgctgtaattatgacggccccactctgtgatgttaagagtatgaagattgctaagagaggaaatccatttctactcaactttattcctgaggttgattatgtaccgctatctgttgttataaccacatttaagagggaaaacgtaaagtatccccttgagggttttggggttcttgtaccttccaaggagcaacaacatggtctcaagacactaggcaccctcttctcttctatgatgtttccagatcgggcaccaaacaatgtttatctctatactacttttgttggtggaagccgaaatagagaacttgcaaaagcctcaaggactgagctgaaagagatagtaacttctgaccttaagcagctgttgggtgctgagggagagccaacatatgtgaatcatctatactggagtaaagcatttccattgtacgggcataactatgattcagtcctagatgcaattgacaaaatggagaaaaatcttcctggattattctatgcaggtaaccacagggggggattgtcagttggcaaagcattatcttctggatgcaatgcagctgatcttgttatatcatatcttgaatccgtctcaactgactccaaaagacattgctga

SEQ ID NO: 40 - protein, 504 aa, Nicotiana sp.
MAPSAGEDKHSSAKRVAVIGAGVSGLAAAYKLKIHGLNVTVFEAEGKAGGKLRSVSQDGLIWDEGANTMTESEGDVTFLIDSLGLREKQQFPLSQNKRYIARNGTPVLLPSNPIDLIKSNFLSTGSKLQMLLEPILWKNKKLSQVSDSHESVSGFFQRHFGKEVVDYLIDPFVAGTCGGDPDSLSMHHSFPELWNLEKRFGSVILGAIRSKLSPKNEKKQGPPKTSANKKRQRGSFSFLGGMQTLTDAICKDLREDELRLNSRVLELSCSCTEDSAIDSWSIISASPHKRQSEEESFDAVIMTAPLCDVKSMKIAKRGNPFLLNFIPEVDYVPLSVVITTFKRENVKYPLEGFGVLVPSKEQQHGLKTLGTLFSSMMFPDRAPNNVYLYTTFVGGSRNRELAKASRTELKEIVTSDLKQLLGAEGEPTYVNHLYWSKAFPLYGHNYDSVLDAIDKMEKNLPGLFYAGNHRGGLSVGKALSSGCNAADLVISYLESVSTDSKRHC

SEQ ID NO: 41 - DNA, 1509 bp, Glycine max
  source 1..1509: mol_type = other DNA
atggcttcctctgcaacagacgataacccaagatctgtaaaaagagtagctgttgttggtgctggggtaagtgggcttgctgcggcttacaaattgaaatcacatggtctggatgtcactgtatttgaagctgagggaagagctggagggaggttgagaagtgtttctcaggatggtctaatttgggatgagggagctaatacaatgactgaaagtgaaattgaggttaaaggtttgattgatgctcttggacttcaagaaaagcagcagtttccaatatcacagcataagcgctatattgtgaaaaatggggcaccacttctggtacccacaaatcctgctgcactactgaagagtaaactgctttctgcacaatcaaagatccatctcatttttgaaccatttatgtggaaaagaagtgacccctctaatgtgtgtgatgaaaattctgtggaaagtgtaggcaggttctttgaacgtcattttggaaaagaggttgtggactatctgattgatccttttgttgggggcactagtgcagcagatcctgaatctctctctatgcgccattctttcccagagctatggaatttggagaaaaggtttggctccattatagccggggcattgcaatctaagttattcgccaaaagggaaaaaactggagaaaataggactgcactaagaaaaaacaaacacaagcgtggttcgttttctttccagggtgggatgcagacactgacagatacattgtgcaaagagcttggcaaagacgaccttaaattaaatgaaaaggttttgacattagcttatggtcatgatggaagttcctcttcacaaaactggtctattactagtgcttctaaccaaagtacacaagatgttgatgcagtaatcatgacggctcctctatataatgtcaaggacatcaagatcacaaaaaggggaactccctttccacttaattttcttcccgaggtaagctacgtgccaatctcagtcatgattactaccttcaaaaaggagaatgtaaagagacctttggagggatttggagttcttgttccttctaaagagcaaaaaaatggtttaaaaacccttggtacacttttttcctctatgatgttcccagatcgtgcacctagtgatttatatctctataccaccttcattggcggaactcaaaacagggaacttgctcaagcttcaactgacgagcttaggaaaattgttacttctgacctgagaaagttgttgggagcagagggggaaccaacatttgttaaccatttctattggagtaaaggctttcctttgtatggacgtaactatgggtcagttcttcaagcaattgataagatagaaaaagatcttcccggatttttctttgcaggtaactacaaaggtggactctcagttggcaaagcaatagcctcaggctgcaaagcagctgatcttgtgatatcctacctcaactctgcttcagacaacacagtgcctgataaatga

SEQ ID NO: 42 - protein, 502 aa, Glycine sp.
MASSATDDNPRSVKRVAVVGAGVSGLAAAYKLKSHGLDVTVFEAEGRAGGRLRSVSQDGLIWDEGANTMTESEIEVKGLIDALGLQEKQQFPISQHKRYIVKNGAPLLVPTNPAALLKSKLLSAQSKIHLIFEPFMWKRSDPSNVCDENSVESVGRFFERHFGKEVVDYLIDPFVGGTSAADPESLSMRHSFPELWNLEKRFGSIIAGALQSKLFAKREKTGENRTALRKNKHKRGSFSFQGGMQTLTDTLCKELGKDDLKLNEKVLTLAYGHDGSSSSQNWSITSASNQSTQDVDAVIMTAPLYNVKDIKITKRGTPFPLNFLPEVSYVPISVMITTFKKENVKRPLEGFGVLVPSKEQKNGLKTLGTLFSSMMFPDRAPSDLYLYTTFIGGTQNRELAQASTDELRKIVTSDLRKLLGAEGEPTFVNHFYWSKGFPLYGRNYGSVLQAIDKIEKDLPGFFFAGNYKGGLSVGKAIASGCKAADLVISYLNSASDNTVPDK

SEQ ID NO: 43 - DNA, 1205 bp, Cucumis sativus
  source 1..1205: mol_type = other DNA
agcttccaaccttccgatcctattctcaccatggtggtggatagtggcttaaaagatgatttagttctgggagacccagatgcacctcgatttgtattgtggaatggaaagctcagaccagtgcctgcgaaacctaatgatctacctttctttgacctgatgagcattggtggaaaaatcagagcaggctttggtgccctgggcattcgccctcctcctccaggtcgagaggaatcagttgaagaatttgtccgtcggaaccttggcaatgaagtttttgaacgtttgatagagccattttgttctggtgtatacgctggtgacccttcaaagctaagcatgaaagcagcttttggtaaggtttggaggctagagcaaaatggtggtagtattattggtgggactttcaaagcacttcaagaaaggaataaaactaccaaaccaccaagagatccgcgtctaccaaagcctaagggccaaactgttggatcttttcggaaaggacttaccatgttgccaaatgctatttctacttgtttggggagtaaagtaaaagtatcttggaagctatctagtatcagtaaagtggatgacggaggttatagtttgacatacgaaacaccagaaggactagtctccatactaagcagaagtgtcatcatgacggttccttcttatattgctggcactctgttgcgtccaatctcggggaaagctgcagatgcactttcaaaattttattatccaccagttgcatcagtgaccatatcatatccaaaaggagcaattaggaaagaatgcttgattgatggtgaactaaaggggtttggtcaattgcaccctcgtagccagggggtgactactttgggaactatatacagctcatcactttttcctaatcgagcgccagatggaagggtattgctcttgaactacattggaggggctactaatactggaattctttctcagacagagagcgagctcatagaagtagttgatcgggatttaagaaaaatcctcataaacccaaacgcagaggatcctctaccattgagcgtgagggtgtggccacaagccattccacagttcttgattggccatctcgatgttctagacaccgccaaggccggactgagagaggctggaatggaggggctatttttaggtggaaactatgtatgcggtgtggccttggggagatg

SEQ ID NO: 44 - protein, 401 aa, Cucumis sp.
SFQPSDPILTMVVDSGLKDDLVLGDPDAPRFVLWNGKLRPVPAKPNDLPFFDLMSIGGKIRAGFGALGIRPPPPGREESVEEFVRRNLGNEVFERLIEPFCSGVYAGDPSKLSMKAAFGKVWRLEQNGGSIIGGTFKALQERNKTTKPPRDPRLPKPKGQTVGSFRKGLTMLPNAISTCLGSKVKVSWKLSSISKVDDGGYSLTYETPEGLVSILSRSVIMTVPSYIAGTLLRPISGKAADALSKFYYPPVASVTISYPKGAIRKECLIDGELKGFGQLHPRSQGVTTLGTIYSSSLFPNRAPDGRVLLLNYIGGATNTGILSQTESELIEVVDRDLRKILINPNAEDPLPLSVRVWPQAIPQFLIGHLDVLDTAKAGLREAGMEGLFLGGNYVCGVALGR

SEQ ID NO: 45 - DNA, 1521 bp, Oryza sativa
  source 1..1521: mol_type = other DNA
atggccgcctccgacgacccccgcggcgggaggtccgtcgccgtcgtcggcgccggcgtcagtgggctcgcggcggcgtacaggctgaggaagcgcggcgtgcaggtgacggtgttcgaggcggccgacagggcgggtgggaagatacggaccaactccgagggcgggttcatctgggacgaaggggccaacaccatgacagagagtgaattggaggcaagcaggcttattgacgatcttggcctacaaggcaaacagcagtatcctaactcacaacacaagcgttacattgtcaaagatggagcaccaacactgattccctcagatcccattgcgctcatgaaaagcactgttctttctacaaaatcaaagctcaagctatttctggaaccatttctctatgagaaatctagcagaaggacctcgggaaaagtgtctgatgaacatttaagtgagagtgtgatttttctgtgtatatgtagagataatcaggttgttgattatcttattgatccatttgtggctggaacaagcggaggagatcctgagtcattatcaattcgtcatgcatttccagcattatggaatttggagaataagtatggctctgtcattgctggtgccatcttgtccaaactatccactaagggtgattcagtgaagacaggaggtgcttcgccagggaaaggaaggaataaacgtgtgtcattttcatttcatggtggaatgcagtcactaatagatgcacttcacaatgaagttggagatggtaacgtgaagcttggtacagaagtgttgtcattggcatgttgctgtgatggagtctcttcttctggtggttggtcaatttctgttgattcaaaagatgctaaagggaaagatctcagaaagaaccaatctttcgatgctgttataatgactgctccattgtctaatgtccagaggatgaagtttacaaaaggtggagttccctttgtgctagactttcttcctaaggtcgattatctaccactatctctcatggtaacagcttttaagaaggaagatgtcaaaaaaccattggaaggatttggtgccttgataccctataaggaacagcaaaagcatggtctcaaaacccttgggaccctcttctcctcgatgatgtttccagatcgagctcctaatgatcaatatctatatacatctttcattggggggagccataatagagacctcgctggggctccaacggctattctgaaacaacttgtgacctctgacctaagaaagctcttgggtgttgagggacaacctacttttgtgaagcatgtacattggagaaatgcttttcctttatatggccagaattatgatctggtactggaagctatagcaaaaatggagaacaatcttccagggttcttttacgcaggaaataacaaggatgggttggctgttggaaatgttatagcttcaggaagcaaggctgctgaccttgtgatctcttatcttgaatcttgcacagatcaggacaattag

SEQ ID NO: 46 - protein, 506 aa, Oryza sativa
MAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESVIFLCICRDNQVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTLFSSMMFPDRAPNDQYLYTSFIGGSHNRDLAGAPTAILKQLVTSDLRKLLGVEGQPTFVKHVHWRNAFPLYGQNYDLVLEAIAKMENNLPGFFYAGNNKDGLAVGNVIASGSKAADLVISYLESCTDQDN

SEQ ID NO: 47 - DNA, 26 bp, synthetic construct
  misc_feature 1..26: note = Forward primer; Cr_PPO1_Fw
  source 1..26: mol_type = other DNA
atgatgttgacccagactcctgggac

SEQ ID NO: 48 - DNA, 25 bp, synthetic construct
  misc_feature 1..25: note = Reverse primer; Cr_PPO1_Rv
  source 1..25: mol_type = other DNA
ttaggccttgactgcggccttggac